SEQUENCE LISTING (285 sequences)

SEQ ID NO: 1 - protein, 7 aa, Artificial Sequence
  Synthetic construct
GYTFTNY

SEQ ID NO: 2 - protein, 6 aa, Artificial Sequence
  Synthetic construct
NTYTGE

SEQ ID NO: 3 - protein, 12 aa, Artificial Sequence
  Synthetic construct
FPYYGNYGAMDY

SEQ ID NO: 4 - protein, 7 aa, Artificial Sequence
  Synthetic construct
GYTFTNY

SEQ ID NO: 5 - protein, 6 aa, Artificial Sequence
  Synthetic construct
NTYTGE

SEQ ID NO: 6 - protein, 12 aa, Artificial Sequence
  Synthetic construct
FPYYGNYGAMDY

SEQ ID NO: 7 - protein, 7 aa, Artificial Sequence
  Synthetic construct
GYTFTNY

SEQ ID NO: 8 - protein, 6 aa, Artificial Sequence
  Synthetic construct
NPYTGE

SEQ ID NO: 9 - protein, 10 aa, Artificial Sequence
  Synthetic construct
SVYGYWYFDV

SEQ ID NO: 10 - protein, 7 aa, Artificial Sequence
  Synthetic construct
GYTFTNC

SEQ ID NO: 11 - protein, 6 aa, Artificial Sequence
  Synthetic construct
NTYTGD

SEQ ID NO: 12 - protein, 12 aa, Artificial Sequence
  Synthetic construct
FPYYGNYGAMDY

SEQ ID NO: 13 - protein, 7 aa, Artificial Sequence
  Synthetic construct
GYTFTHY

SEQ ID NO: 14 - protein, 6 aa, Artificial Sequence
  Synthetic construct
ITYTGE

SEQ ID NO: 15 - protein, 9 aa, Artificial Sequence
  Synthetic construct
YYGSNYFDY

SEQ ID NO: 16 - protein, 7 aa, Artificial Sequence
  Synthetic construct
GYTFTNY

SEQ ID NO: 17 - protein, 7 aa, Artificial Sequence
  Synthetic construct
INTYTGE

SEQ ID NO: 18 - protein, 12 aa, Artificial Sequence
  Synthetic construct
FPYYGNYGAMDY

SEQ ID NO: 19 - protein, 9 aa, Artificial Sequence
  Synthetic construct
GFSLSTSGM

SEQ ID NO: 20 - protein, 5 aa, Artificial Sequence
  Synthetic construct
YWDDD

SEQ ID NO: 21 - protein, 9 aa, Artificial Sequence
  Synthetic construct
RGNYYAMDY

SEQ ID NO: 22 - protein, 7 aa, Artificial Sequence
  Synthetic construct
GYTFTSY

SEQ ID NO: 23 - protein, 6 aa, Artificial Sequence
  Synthetic construct
NPSNGR

SEQ ID NO: 24 - protein, 9 aa, Artificial Sequence
  Synthetic construct
YYDNDDFDY

SEQ ID NO: 25 - protein, 7 aa, Artificial Sequence
  Synthetic construct
GYTFTNY

SEQ ID NO: 26 - protein, 6 aa, Artificial Sequence
  Synthetic construct
NTYTGE

SEQ ID NO: 27 - protein, 9 aa, Artificial Sequence
  Synthetic construct
GGYYRYALY

SEQ ID NO: 28 - protein, 10 aa, Artificial Sequence
  Synthetic construct
KASGYTFTSY

SEQ ID NO: 29 - protein, 6 aa, Artificial Sequence
  Synthetic construct
NPSNGR

SEQ ID NO: 30 - protein, 9 aa, Artificial Sequence
  Synthetic construct
YYDNDDFDY

SEQ ID NO: 31 - protein, 7 aa, Artificial Sequence
  Synthetic construct
GYTFTNY

SEQ ID NO: 32 - protein, 6 aa, Artificial Sequence
  Synthetic construct
NPYTGE

SEQ ID NO: 33 - protein, 10 aa, Artificial Sequence
  Synthetic construct
SVYGYWYLDV

SEQ ID NO: 34 - protein, 8 aa, Artificial Sequence
  Synthetic construct
GYSITSDY

SEQ ID NO: 35 - protein, 5 aa, Artificial Sequence
  Synthetic construct
SYSGN

SEQ ID NO: 36 - protein, 9 aa, Artificial Sequence
  Synthetic construct
AATGYVMDY

SEQ ID NO: 37 - protein, 16 aa, Artificial Sequence
  Synthetic construct
RSSKSLLNSNGINYLY

SEQ ID NO: 38 - protein, 7 aa, Artificial Sequence
  Synthetic construct
QMSNLAS

SEQ ID NO: 39 - protein, 9 aa, Artificial Sequence
  Synthetic construct
AQNLELPCT

SEQ ID NO: 40 - protein, 11 aa, Artificial Sequence
  Synthetic construct
KANKDGNNNLN

SEQ ID NO: 41 - protein, 7 aa, Artificial Sequence
  Synthetic construct
GASNLES

SEQ ID NO: 42 - protein, 9 aa, Artificial Sequence
  Synthetic construct
QQNSNFPYT

SEQ ID NO: 43 - protein, 5 aa, Artificial Sequence
  Synthetic construct
  misc_feature (1)..(5): Xaa can be any naturally occurring amino acid
XXXXX

SEQ ID NO: 44 - protein, 5 aa, Artificial Sequence
  Synthetic construct
  misc_feature (1)..(5): Xaa can be any naturally occurring amino acid
XXXXX

SEQ ID NO: 45 - protein, 5 aa, Artificial Sequence
  Synthetic construct
  misc_feature (1)..(5): Xaa can be any naturally occurring amino acid
XXXXX

SEQ ID NO: 46 - protein, 15 aa, Artificial Sequence
  Synthetic construct
RSSKSLLHSNGITYL

SEQ ID NO: 47 - protein, 7 aa, Artificial Sequence
  Synthetic construct
QMSNLAS

SEQ ID NO: 48 - protein, 9 aa, Artificial Sequence
  Synthetic construct
AQNLELPWT

SEQ ID NO: 49 - protein, 14 aa, Artificial Sequence
  Synthetic construct
RASESVDSYGNSFM

SEQ ID NO: 50 - protein, 7 aa, Artificial Sequence
  Synthetic construct
RASNLES

SEQ ID NO: 51 - protein, 9 aa, Artificial Sequence
  Synthetic construct
QQSNEDPYT

SEQ ID NO: 52 - protein, 15 aa, Artificial Sequence
  Synthetic construct
RSSKSLLHSNGVTYL

SEQ ID NO: 53 - protein, 7 aa, Artificial Sequence
  Synthetic construct
QMSNLAS

SEQ ID NO: 54 - protein, 9 aa, Artificial Sequence
  Synthetic construct
AQNLELPCT

SEQ ID NO: 55 - protein, 9 aa, Artificial Sequence
  Synthetic construct
SASSSVSYM

SEQ ID NO: 56 - protein, 7 aa, Artificial Sequence
  Synthetic construct
LTSILAS

SEQ ID NO: 57 - protein, 9 aa, Artificial Sequence
  Synthetic construct
QQWSSYPPT

SEQ ID NO: 58 - protein, 16 aa, Artificial Sequence
  Synthetic construct
KSSQSLLDSDGKTYLN

SEQ ID NO: 59 - protein, 7 aa, Artificial Sequence
  Synthetic construct
LVSKLDS

SEQ ID NO: 60 - protein, 9 aa, Artificial Sequence
  Synthetic construct
WQGTHFPHT

SEQ ID NO: 61 - protein, 11 aa, Artificial Sequence
  Synthetic construct
KASEDIDNRLG

SEQ ID NO: 62 - protein, 7 aa, Artificial Sequence
  Synthetic construct
GATSLET

SEQ ID NO: 63 - protein, 9 aa, Artificial Sequence
  Synthetic construct
QQYWSTPYT

SEQ ID NO: 64 - protein, 5 aa, Artificial Sequence
  Synthetic construct
  misc_feature (1)..(5): Xaa can be any naturally occurring amino acid
XXXXX

SEQ ID NO: 65 - protein, 5 aa, Artificial Sequence
  Synthetic construct
  misc_feature (1)..(5): Xaa can be any naturally occurring amino acid
XXXXX

SEQ ID NO: 66 - protein, 5 aa, Artificial Sequence
  Synthetic construct
  misc_feature (1)..(5): Xaa can be any naturally occurring amino acid
XXXXX

SEQ ID NO: 67 - protein, 5 aa, Artificial Sequence
  Synthetic construct
  misc_feature (1)..(5): Xaa can be any naturally occurring amino acid
XXXXX

SEQ ID NO: 68 - protein, 5 aa, Artificial Sequence
  Synthetic construct
  misc_feature (1)..(5): Xaa can be any naturally occurring amino acid
XXXXX

SEQ ID NO: 69 - protein, 5 aa, Artificial Sequence
  Synthetic construct
  misc_feature (1)..(5): Xaa can be any naturally occurring amino acid
XXXXX

SEQ ID NO: 70 - protein, 10 aa, Artificial Sequence
  Synthetic construct
SASSSVSYMN

SEQ ID NO: 71 - protein, 7 aa, Artificial Sequence
  Synthetic construct
GISYLAS

SEQ ID NO: 72 - protein, 9 aa, Artificial Sequence
  Synthetic construct
QQRSSYPLT

SEQ ID NO: 73 - protein, 7 aa, Artificial Sequence
  Synthetic construct
GYTFTNY

SEQ ID NO: 74 - protein, 6 aa, Artificial Sequence
  Synthetic construct
NTYTGE

SEQ ID NO: 75 - protein, 12 aa, Artificial Sequence
  Synthetic construct
FPYYGNYGAMDY

SEQ ID NO: 76 - protein, 5 aa, Artificial Sequence
  Synthetic construct
  misc_feature (1)..(5): Xaa can be any naturally occurring amino acid
XXXXX

SEQ ID NO: 77 - protein, 5 aa, Artificial Sequence
  Synthetic construct
  misc_feature (1)..(5): Xaa can be any naturally occurring amino acid
XXXXX

SEQ ID NO: 78 - protein, 5 aa, Artificial Sequence
  Synthetic construct
  misc_feature (1)..(5): Xaa can be any naturally occurring amino acid
XXXXX

SEQ ID NO: 79 - protein, 7 aa, Artificial Sequence
  Synthetic construct
GYTFTDY

SEQ ID NO: 80 - protein, 6 aa, Artificial Sequence
  Synthetic construct
DPETGG

SEQ ID NO: 81 - protein, 9 aa, Artificial Sequence
  Synthetic construct
AVVATDYFD

SEQ ID NO: 82 - protein, 5 aa, Artificial Sequence
  Synthetic construct
  misc_feature (1)..(5): Xaa can be any naturally occurring amino acid
XXXXX

SEQ ID NO: 83 - protein, 5 aa, Artificial Sequence
  Synthetic construct
  misc_feature (1)..(5): Xaa can be any naturally occurring amino acid
XXXXX

SEQ ID NO: 84 - protein, 5 aa, Artificial Sequence
  Synthetic construct
  misc_feature (1)..(5): Xaa can be any naturally occurring amino acid
XXXXX

SEQ ID NO: 85 - protein, 7 aa, Artificial Sequence
  Synthetic construct
GFNIKDT

SEQ ID NO: 86 - protein, 6 aa, Artificial Sequence
  Synthetic construct
DPATGN

SEQ ID NO: 87 - protein, 12 aa, Artificial Sequence
  Synthetic construct
HYYGGTYYPMDY

SEQ ID NO: 88 - protein, 7 aa, Artificial Sequence
  Synthetic construct
GYTFTNY

SEQ ID NO: 89 - protein, 6 aa, Artificial Sequence
  Synthetic construct
NTYTGE

SEQ ID NO: 90 - protein, 12 aa, Artificial Sequence
  Synthetic construct
RISTMITTWFAY

SEQ ID NO: 91 - protein, 7 aa, Artificial Sequence
  Synthetic construct
GFSFTGY

SEQ ID NO: 92 - protein, 6 aa, Artificial Sequence
  Synthetic construct
NPYNGV

SEQ ID NO: 93 - protein, 9 aa, Artificial Sequence
  Synthetic construct
LDYDSSFDY

SEQ ID NO: 94 - protein, 7 aa, Artificial Sequence
  Synthetic construct
GYTFSNY

SEQ ID NO: 95 - protein, 6 aa, Artificial Sequence
  Synthetic construct
LPGSGR

SEQ ID NO: 96 - protein, 13 aa, Artificial Sequence
  Synthetic construct
YYRYDGGYAMDYW

SEQ ID NO: 97 - protein, 8 aa, Artificial Sequence
  Synthetic construct
YGYTFTNY

SEQ ID NO: 98 - protein, 6 aa, Artificial Sequence
  Synthetic construct
NTYTGE

SEQ ID NO: 99 - protein, 14 aa, Artificial Sequence
  Synthetic construct
VRRVSTMITTWFAY

SEQ ID NO: 100 - protein, 7 aa, Artificial Sequence
  Synthetic construct
GYSFTGY

SEQ ID NO: 101 - protein, 6 aa, Artificial Sequence
  Synthetic construct
NPYNGA

SEQ ID NO: 102 - protein, 9 aa, Artificial Sequence
  Synthetic construct
DDYEGAMDY

SEQ ID NO: 103 - protein, 8 aa, Artificial Sequence
  Synthetic construct
GYSITSGD

SEQ ID NO: 104 - protein, 5 aa, Artificial Sequence
  Synthetic construct
HYSGS

SEQ ID NO: 105 - protein, 8 aa, Artificial Sequence
  Synthetic construct
WFYYAMDY

SEQ ID NO: 106 - protein, 5 aa, Artificial Sequence
  Synthetic construct
  misc_feature (1)..(5): Xaa can be any naturally occurring amino acid
XXXXX

SEQ ID NO: 107 - protein, 5 aa, Artificial Sequence
  Synthetic construct
  misc_feature (1)..(5): Xaa can be any naturally occurring amino acid
XXXXX

SEQ ID NO: 108 - protein, 5 aa, Artificial Sequence
  Synthetic construct
  misc_feature (1)..(5): Xaa can be any naturally occurring amino acid
XXXXX

SEQ ID NO: 109 - protein, 11 aa, Artificial Sequence
  Synthetic construct
KASQNVGTNVA

SEQ ID NO: 110 - protein, 7 aa, Artificial Sequence
  Synthetic construct
SASYRYS

SEQ ID NO: 111 - protein, 8 aa, Artificial Sequence
  Synthetic construct
QYNSYPYT

SEQ ID NO: 112 - protein, 5 aa, Artificial Sequence
  Synthetic construct
  misc_feature (1)..(5): Xaa can be any naturally occurring amino acid
XXXXX

SEQ ID NO: 113 - protein, 5 aa, Artificial Sequence
  Synthetic construct
  misc_feature (1)..(5): Xaa can be any naturally occurring amino acid
XXXXX

SEQ ID NO: 114 - protein, 5 aa, Artificial Sequence
  Synthetic construct
  misc_feature (1)..(5): Xaa can be any naturally occurring amino acid
XXXXX

SEQ ID NO: 115 - protein, 8 aa, Artificial Sequence
  Synthetic construct
QDINKYIA

SEQ ID NO: 116 - protein, 7 aa, Artificial Sequence
  Synthetic construct
YTSTLQP

SEQ ID NO: 117 - protein, 8 aa, Artificial Sequence
  Synthetic construct
LQYDNLWT

SEQ ID NO: 118 - protein, 17 aa, Artificial Sequence
  Synthetic construct
KSSQSLLNSRTRKNYLA

SEQ ID NO: 119 - protein, 7 aa, Artificial Sequence
  Synthetic construct
WASTRES

SEQ ID NO: 120 - protein, 8 aa, Artificial Sequence
  Synthetic construct
KQSYNLWT

SEQ ID NO: 121 - protein, 11 aa, Artificial Sequence
  Synthetic construct
RASENIYSYLA

SEQ ID NO: 122 - protein, 7 aa, Artificial Sequence
  Synthetic construct
YAKTLAE

SEQ ID NO: 123 - protein, 9 aa, Artificial Sequence
  Synthetic construct
QHHYGTPYT

SEQ ID NO: 124 - protein, 5 aa, Artificial Sequence
  Synthetic construct
  misc_feature (1)..(5): Xaa can be any naturally occurring amino acid
XXXXX

SEQ ID NO: 125 - protein, 6 aa, Artificial Sequence
  Synthetic construct
  misc_feature (1)..(6): Xaa can be any naturally occurring amino acid
XXXXXX

SEQ ID NO: 126 - protein, 5 aa, Artificial Sequence
  Synthetic construct
  misc_feature (1)..(5): Xaa can be any naturally occurring amino acid
XXXXX

SEQ ID NO: 127 - protein, 11 aa, Artificial Sequence
  Synthetic construct
RASQEISGYLN

SEQ ID NO: 128 - protein, 7 aa, Artificial Sequence
  Synthetic construct
AASTLDS

SEQ ID NO: 129 - protein, 9 aa, Artificial Sequence
  Synthetic construct
LQYAGYPYT

SEQ ID NO: 130 - protein, 11 aa, Artificial Sequence
  Synthetic construct
RVSENIYSYLA

SEQ ID NO: 131 - protein, 7 aa, Artificial Sequence
  Synthetic construct
YAKTLAE

SEQ ID NO: 132 - protein, 9 aa, Artificial Sequence
  Synthetic construct
QHHYDIPYT

SEQ ID NO: 133 - protein, 17 aa, Artificial Sequence
  Synthetic construct
KSSQSVLYISNQKNYLA

SEQ ID NO: 134 - protein, 7 aa, Artificial Sequence
  Synthetic construct
WASTRES

SEQ ID NO: 135 - protein, 8 aa, Artificial Sequence
  Synthetic construct
HQYFSSYT

SEQ ID NO: 136 - protein, 12 aa, Artificial Sequence
  Synthetic construct
RASSSVSSSYLH

SEQ ID NO: 137 - protein, 7 aa, Artificial Sequence
  Synthetic construct
STSNLAS

SEQ ID NO: 138 - protein, 9 aa, Artificial Sequence
  Synthetic construct
QQYSGFPLT

SEQ ID NO: 139 - protein, 7 aa, Artificial Sequence
  Synthetic construct
GFSLTSY

SEQ ID NO: 140 - protein, 5 aa, Artificial Sequence
  Synthetic construct
WAGGS

SEQ ID NO: 141 - protein, 11 aa, Artificial Sequence
  Synthetic construct
DGSRYEGYFDY

SEQ ID NO: 142 - protein, 7 aa, Artificial Sequence
  Synthetic construct
GYTFTNY

SEQ ID NO: 143 - protein, 6 aa, Artificial Sequence
  Synthetic construct
NTYTGD

SEQ ID NO: 144 - protein, 12 aa, Artificial Sequence
  Synthetic construct
RISTMITTWFAY

SEQ ID NO: 145 - protein, 7 aa, Artificial Sequence
  Synthetic construct
GYTFTNY

SEQ ID NO: 146 - protein, 6 aa, Artificial Sequence
  Synthetic construct
NTYTGD

SEQ ID NO: 147 - protein, 12 aa, Artificial Sequence
  Synthetic construct
RISTMITTWFAY

SEQ ID NO: 148 - protein, 7 aa, Artificial Sequence
  Synthetic construct
GYTFTNY

SEQ ID NO: 149 - protein, 6 aa, Artificial Sequence
  Synthetic construct
NTYTGD

SEQ ID NO: 150 - protein, 12 aa, Artificial Sequence
  Synthetic construct
RISTMITTWFAY

SEQ ID NO: 151 - protein, 5 aa, Artificial Sequence
  Synthetic construct
  misc_feature (1)..(5): Xaa can be any naturally occurring amino acid
XXXXX

SEQ ID NO: 152 - protein, 5 aa, Artificial Sequence
  Synthetic construct
  misc_feature (1)..(5): Xaa can be any naturally occurring amino acid
XXXXX

SEQ ID NO: 153 - protein, 5 aa, Artificial Sequence
  Synthetic construct
  misc_feature (1)..(5): Xaa can be any naturally occurring amino acid
XXXXX

SEQ ID NO: 154 - protein, 7 aa, Artificial Sequence
  Synthetic construct
GFTFSSF

SEQ ID NO: 155 - protein, 5 aa, Artificial Sequence
  Synthetic construct
SSGSS

SEQ ID NO: 156 - protein, 12 aa, Artificial Sequence
  Synthetic construct
QGLRYYGYAMDY

SEQ ID NO: 157 - protein, 7 aa, Artificial Sequence
  Synthetic construct
GYTFTSY

SEQ ID NO: 158 - protein, 6 aa, Artificial Sequence
  Synthetic construct
NPSTGY

SEQ ID NO: 159 - protein, 11 aa, Artificial Sequence
  Synthetic construct
WVITTGKYFDV

SEQ ID NO: 160 - protein, 7 aa, Artificial Sequence
  Synthetic construct
GYTFSNY

SEQ ID NO: 161 - protein, 6 aa, Artificial Sequence
  Synthetic construct
LPGSGR

SEQ ID NO: 162 - protein, 9 aa, Artificial Sequence
  Synthetic construct
QLGVRAMDY

SEQ ID NO: 163 - protein, 7 aa, Artificial Sequence
  Synthetic construct
GYTFSNY

SEQ ID NO: 164 - protein, 6 aa, Artificial Sequence
  Synthetic construct
LPGSGR

SEQ ID NO: 165 - protein, 12 aa, Artificial Sequence
  Synthetic construct
YYRYDGGYAMDY

SEQ ID NO: 166 - protein, 7 aa, Artificial Sequence
  Synthetic construct
GFAFSSY

SEQ ID NO: 167 - protein, 6 aa, Artificial Sequence
  Synthetic construct
SKGGGS

SEQ ID NO: 168 - protein, 9 aa, Artificial Sequence
  Synthetic construct
HAGNYAMDY

SEQ ID NO: 169 - protein, 17 aa, Artificial Sequence
  Synthetic construct
KSSQSLLNSNNQKNYLA

SEQ ID NO: 170 - protein, 7 aa, Artificial Sequence
  Synthetic construct
FASTRES

SEQ ID NO: 171 - protein, 9 aa, Artificial Sequence
  Synthetic construct
QQHYSTPLT

SEQ ID NO: 172 - protein, 5 aa, Artificial Sequence
  Synthetic construct
  misc_feature (1)..(5): Xaa can be any naturally occurring amino acid
XXXXX

SEQ ID NO: 173 - protein, 5 aa, Artificial Sequence
  Synthetic construct
  misc_feature (1)..(5): Xaa can be any naturally occurring amino acid
XXXXX

SEQ ID NO: 174 - protein, 5 aa, Artificial Sequence
  Synthetic construct
  misc_feature (1)..(5): Xaa can be any naturally occurring amino acid
XXXXX

SEQ ID NO: 175 - protein, 11 aa, Artificial Sequence
  Synthetic construct
RASENIYSYLA

SEQ ID NO: 176 - protein, 7 aa, Artificial Sequence
  Synthetic construct
NAKTLTE

SEQ ID NO: 177 - protein, 10 aa, Artificial Sequence
  Synthetic construct
QHYQVTPYTF

SEQ ID NO: 178 - protein, 11 aa, Artificial Sequence
  Synthetic construct
RASENIYSYLA

SEQ ID NO: 179 - protein, 7 aa, Artificial Sequence
  Synthetic construct
NAKTLTE

SEQ ID NO: 180 - protein, 10 aa, Artificial Sequence
  Synthetic construct
QHYQVTPYTF

SEQ ID NO: 181 - protein, 16 aa, Artificial Sequence
  Synthetic construct
KSSQSLLDSDGKTYLN

SEQ ID NO: 182 - protein, 7 aa, Artificial Sequence
  Synthetic construct
LVSKLDS

SEQ ID NO: 183 - protein, 9 aa, Artificial Sequence
  Synthetic construct
WQDTHFPQT

SEQ ID NO: 184 - protein, 11 aa, Artificial Sequence
  Synthetic construct
RASQSIGTIIH

SEQ ID NO: 185 - protein, 7 aa, Artificial Sequence
  Synthetic construct
YASESIS

SEQ ID NO: 186 - protein, 9 aa, Artificial Sequence
  Synthetic construct
QQTNSWPLT

SEQ ID NO: 187 - protein, 15 aa, Artificial Sequence
  Synthetic construct
RASESVDYYGNSFIH

SEQ ID NO: 188 - protein, 7 aa, Artificial Sequence
  Synthetic construct
RASNLES

SEQ ID NO: 189 - protein, 9 aa, Artificial Sequence
  Synthetic construct
QQSNEDPLT

SEQ ID NO: 190 - protein, 5 aa, Artificial Sequence
  Synthetic construct
  misc_feature (1)..(5): Xaa can be any naturally occurring amino acid
XXXXX

SEQ ID NO: 191 - protein, 5 aa, Artificial Sequence
  Synthetic construct
  misc_feature (1)..(5): Xaa can be any naturally occurring amino acid
XXXXX

SEQ ID NO: 192 - protein, 5 aa, Artificial Sequence
  Synthetic construct
  misc_feature (1)..(5): Xaa can be any naturally occurring amino acid
XXXXX

SEQ ID NO: 193 - protein, 16 aa, Artificial Sequence
  Synthetic construct
RSSKSLLHSNGITYLY

SEQ ID NO: 194 - protein, 7 aa, Artificial Sequence
  Synthetic construct
QMSNLAS

SEQ ID NO: 195 - protein, 9 aa, Artificial Sequence
  Synthetic construct
AQNLELPYT

SEQ ID NO: 196 - protein, 11 aa, Artificial Sequence
  Synthetic construct
KASQDINSYLS

SEQ ID NO: 197 - protein, 7 aa, Artificial Sequence
  Synthetic construct
RANRLVD

SEQ ID NO: 198 - protein, 9 aa, Artificial Sequence
  Synthetic construct
LQYDEFPYT

SEQ ID NO: 199 - protein, 7 aa, Artificial Sequence
  Synthetic construct
GYSFTGY

SEQ ID NO: 200 - protein, 6 aa, Artificial Sequence
  Synthetic construct
NCYNGA

SEQ ID NO: 201 - protein, 14 aa, Artificial Sequence
  Synthetic construct
AGIHYDYDEAWFAY

SEQ ID NO: 202 - protein, 14 aa, Artificial Sequence
  Synthetic construct
RSSTGPVIIRNYAN

SEQ ID NO: 203 - protein, 7 aa, Artificial Sequence
  Synthetic construct
GTNNRAP

SEQ ID NO: 204 - protein, 9 aa, Artificial Sequence
  Synthetic construct
ALWYNNHWV

SEQ ID NO: 205 - protein, 121 aa, Artificial Sequence
  Synthetic construct
QIQLVQSGPELKKPGETVKISCKASGYTFTNYGMNWVKQAPGKGLKWMGWINTYTGEPTYADDFEGRFALSLETSASTVYLQINNLKNEDTGTYFCARFPYYGNYGAMDYWGQGTSVTVSS

SEQ ID NO: 206 - protein, 112 aa, Artificial Sequence
  Synthetic construct
DIVMTQAAFSNPVTLGTSASISCRSSKSLLNSNGINYLYWYLQKPGQSPQLLIYQMSNLASGVPDRFSSSGSGTDFTLRISRVEAEDVGVYYCAQNLELPCTFGGGTKLEIK

SEQ ID NO: 207 - protein, 121 aa, Artificial Sequence
  Synthetic construct
QIQLVQSGPELKKPGETVKISCKASGYTFTNYGMNWVKQAPGKGLKWMGWINTYTGEPTYADDFEGRFALSLETSASTVYLQINNLKNEDTATYFCARFPYYGNYGAMDYWGQGTSVTVSS

SEQ ID NO: 208 - protein, 107 aa, Artificial Sequence
  Synthetic construct
QTPMTQSSTIGSASSGNRKSITSKANKDGNNNLNWFQQKPGQSPKLLIYGASNLESGVPARFSGSGSGTDFTFTISSVEAEDVATYYCQQNSNFPYTFGGGTKLEIK

SEQ ID NO: 209 - protein, 119 aa, Artificial Sequence
  Synthetic construct
QIQLVQSGPELKKPGETVKISCKASGYTFTNYGMNWVKQAPGKGLKWMGWINPYTGEPRYADDFKGRFAFSLETSASTAFLQISNLKNEDMATYFCARSVYGYWYFDVWGAGTTVTVSS

SEQ ID NO: 210 - protein, 10 aa, Artificial Sequence
  Synthetic construct
  misc_feature (1)..(10): Xaa can be any naturally occurring amino acid
XXXXXXXXXX

SEQ ID NO: 211 - protein, 121 aa, Artificial Sequence
  Synthetic construct
QIQLVQSGPELKKPGETVKISCKASGYTFTNCGMNWVKQAPGKGLKWMGWINTYTGDPTYADDFEGRFAFSLETSASTAYLQINNLKNEDTATYFCARFPYYGNYGAMDYWGQGTSVTVSS

SEQ ID NO: 212 - protein, 112 aa, Artificial Sequence
  Synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 213 - protein, 119 aa, Artificial Sequence
  Synthetic construct
QIQLVQSGPELKKPGETVKISCKASGYTFTHYGMTWVKQAPGKGLKWMGWIITYTGEPTYADDFKGRFAFSLETSASTAYLQINNLKNEDMATYFCARRYYGSNYFDYWGQGTTLTVSS

SEQ ID NO: 214 - protein, 112 aa, Artificial Sequence
  Synthetic construct
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGPTGNKTG

SEQ ID NO: 215 - protein, 121 aa, Artificial Sequence
  Synthetic construct
QIQLVQSGPELKKPGETVKISCKASGYTFTNYGMNWVKQAPGKGLKWMGWINTYTGEPTYADDFKGRFAFSLETSASTAYLQINNLKNEDTVTYFCARFPYYGNYGAMDYWGQGTSVTVSS

SEQ ID NO: 216 - protein, 112 aa, Artificial Sequence
  Synthetic construct
DIVMTQAAFSNPVTLGTSASISCRSSKSLLHSNGVTYLYWYLQKPGQSPQLLIYQMSNLASGVPDRFSSSGSGTDFTLRISRVEAEDVGVYYCAQNLELPCTFGGGTKLEIK

SEQ ID NO: 217 - protein, 119 aa, Artificial Sequence
  Synthetic construct
QVTLKESGPGILQSSQTLSLTCSLFGFSLSTSGMGVSWIRQPSGKGLEWLAHIYWDDDKRYNPSLKSRLTISKGTSSNQVFLKITSVDTADTATYYCARRGNYYAMDYWGQGTSVTVSS

SEQ ID NO: 218 - protein, 106 aa, Artificial Sequence
  Synthetic construct
QIVLTQSPALMSASPGEKVTMTCSASSSVSYMYWYQQKPRSSPKPWIYLTSILASGVPARFSGSGSGTSYSLTISSMEAEDAATYYCQQWSSYPPTFGGGTKLEMK

SEQ ID NO: 219 - protein, 118 aa, Artificial Sequence
  Synthetic construct
QVQLQQPGAELVKPGASVKLSCKASGYTFTSYWMHWVKQRPGQGLEWIGEINPSNGRTNYIEKFKIKATLTVDKSSSTAYMQLSSLTSEDSAVYYCARYYDNDDFDYWGQGTTLTVSS

SEQ ID NO: 220 - protein, 110 aa, Artificial Sequence
  Synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGPSWK

SEQ ID NO: 221 - protein, 118 aa, Artificial Sequence
  Synthetic construct
LIQLVQSGPEVKKPGETVKISCKFSGYTFTNYGMTWVKQAPGKGLKWMGWINTYTGEPTYADDFKGRFAFSLETSASTAYLQINNLKNEDTATYFCATGGYYRYALYWGQGTLVTVSA

SEQ ID NO: 222 - protein, 107 aa, Artificial Sequence
  Synthetic construct
DIQMTQSSSSFSVSLGDRVTITCKASEDIDNRLGWYQQKPGNAPRLLISGATSLETGVPSRFSGSGSGKDYTLSITSLQTEDVATYYCQQYWSTPYTFGGGTKLEIK

SEQ ID NO: 223 - protein, 118 aa, Artificial Sequence; identical to SEQ ID NO: 219
  Synthetic construct

SEQ ID NO: 224 - protein, 10 aa, Artificial Sequence
  Synthetic construct
  misc_feature (1)..(10): Xaa can be any naturally occurring amino acid
XXXXXXXXXX

SEQ ID NO: 225 - protein, 119 aa, Artificial Sequence
  Synthetic construct
QIQLVQSGPELKKPGETVKISCKASGYTFTNYGMNWVKQAPGKGLKWMGWINPYTGEPRYADDFKGRFAFSLETSASTAFLQISNLKNEDMATYFCARSVYGYWYLDVWGAGTTVTVSS

SEQ ID NO: 226 - protein, 10 aa, Artificial Sequence
  Synthetic construct
  misc_feature (1)..(10): Xaa can be any naturally occurring amino acid
XXXXXXXXXX

SEQ ID NO: 227 - protein, 118 aa, Artificial Sequence
  Synthetic construct
DVQLQESGPGLMKPSQSLSLTCTVTGYSITSDYAWNWIRQFPGNKLEWMGYISYSGNPNYNPSLKSRISITRDTSKNQFFLQLNSVTTEDTATYYCAKAATGYVMDYWGQGTSVTVSS

SEQ ID NO: 228 - protein, 106 aa, Artificial Sequence
  Synthetic construct
EILLTQSPAIIAASPGEKVTITCSASSSVSYMNWYQQKPGSSPKIWIYGISYLASGVPARFSGSGSGTSFSFTINSMEAEDVATYYCQQRSSYPLTFGAGTKLELK

SEQ ID NO: 229 - protein, 121 aa, Artificial Sequence; identical to SEQ ID NO: 207
  Synthetic construct

SEQ ID NO: 230 - protein, 10 aa, Artificial Sequence
  Synthetic construct
  misc_feature (1)..(10): Xaa can be any naturally occurring amino acid
XXXXXXXXXX

SEQ ID NO: 231 - protein, 10 aa, Artificial Sequence
  Synthetic construct
  misc_feature (1)..(10): Xaa can be any naturally occurring amino acid
XXXXXXXXXX

SEQ ID NO: 232 - protein, 107 aa, Artificial Sequence
  Synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 233 - protein, 119 aa, Artificial Sequence
  Synthetic construct
QVQLQQSGAELVRPGASVTLSCKASGYTFTDYEMHWVKQTPVHGLEWIGVIDPETGGTAYNQKFKGKATLTADKSSSTAYMELRSLTSEDSAVYYCTRAVVATDYFDYWGQGTTLTVSS

SEQ ID NO: 234 - protein, 8 aa, Artificial Sequence
  Synthetic construct
  misc_feature (1)..(8): Xaa can be any naturally occurring amino acid
XXXXXXXX

SEQ ID NO: 235 - protein, 10 aa, Artificial Sequence
  Synthetic construct
  misc_feature (1)..(10): Xaa can be any naturally occurring amino acid
XXXXXXXXXX

SEQ ID NO: 236 - protein, 106 aa, Artificial Sequence
  Synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKLEIK

SEQ ID NO: 237 - protein, 121 aa, Artificial Sequence
  Synthetic construct
EVQLQQSGAELVKPGASVKLSCTASGFNIKDTYMHWVKQRPEQGLEWIGRIDPATGNTKYDPKFQGKATMTADPSSNTAYLQLSSLTSDDTAVYYCSLHYYGGTYYPMDYWGQGTSVTVSS

SEQ ID NO: 238 - protein, 112 aa, Artificial Sequence
  Synthetic construct
DIVMSQSPSSLAVSAGEKVTMSCKSSQSLLNSRTRKNYLAWYQQKPGQSPKLLIYWASTRESGVPDRFTGSGFGTDFTLTISTVQAEDLAVYYCKQSYNLWTFGGGTKLEIK

SEQ ID NO: 239 - protein, 121 aa, Artificial Sequence
  Synthetic construct
QIQLVQSGPELKKPGETVKISCKASGYTFTNYGMNWVKQAPGKGLKWMGWINTYTGEPTYADDFKGRFAFSLETSASTAFLQINNLKNEDMATYFCTRRISTMITTWFAYWGQGTLVTVSA

SEQ ID NO: 240 - protein, 107 aa, Artificial Sequence
  Synthetic construct
DIQMTQSPASLSASVGETVTITCRASENIYSYLAWYQQKQGKSPQLLVYYAKTLAEGVPSRFSGSGSGTQFSLKINSLQPEDFGSYYCQHHYGTPYTFGGGTKLEIK

SEQ ID NO: 241 - protein, 118 aa, Artificial Sequence
  Synthetic construct
EVQLQQSGPELVKPGASVKISCKASGFSFTGYYMHWVKQSHVKSLEWIGHINPYNGVTRYNQNFKDKASLTVDKSSNTAYMELHSLTSEDSAVYYCSRLDYDSSFDYWGQGTSVTVSS

SEQ ID NO: 242 - protein, 10 aa, Artificial Sequence
  Synthetic construct
  misc_feature (1)..(10): Xaa can be any naturally occurring amino acid
XXXXXXXXXX

SEQ ID NO: 243 - protein, 121 aa, Artificial Sequence
  Synthetic construct
QVQLQQSGAELMKPGASVKISCKATGYTFSNYWIEWVKQRPGHGLEWIGEILPGSGRTNYNEKFKGKATFTADTSSNTAYMQLSSLTSEDSAVYYCAIYYRYDGGYAMDYWGQGTSVTVSS

SEQ ID NO: 244 - protein, 106 aa, Artificial Sequence
  Synthetic construct
PDDPSSPSSLSSYLGERVSLTCRASQEISGYLNWLQQKPDGTIKRLIYAASTLDSGVPKRFSGSRSGSDYSLTISSLESEDFADYYCLQYAGYPYTFGGGTKLEIK

SEQ ID NO: 245 - protein, 121 aa, Artificial Sequence
  Synthetic construct
QIQLVQSGPELKKPGETVKISCKAYGYTFTNYGMNWVKQAPGKGLKWMGWINTYTGEPTYADDFKGRFAFSLETSASTAYLQINNLKNEDMATYFCVRRVSTMITTWFAYWGQGTLVTVSA

SEQ ID NO: 246 - protein, 107 aa, Artificial Sequence
  Synthetic construct
DIQMTQSPASLSASVGETVTITCRVSENIYSYLAWYQQKQGKSPQLLVYYAKTLAEGVPSRFSGSGSGTQFSLKINSLQPEDFGSYYCQHHYDIPYTFGGGTKLEIK

SEQ ID NO: 247 - protein, 118 aa, Artificial Sequence
  Synthetic construct
EVQLQQSGPELVKPGASVKISCKASGYSFTGYYMHWVKQSHVKSLEWIGRINPYNGATIYNQNFKDKASLTVDKSSSTAYMELHSLTSEDSAVYYCARDDYEGAMDYWGQGTSVTVSS

SEQ ID NO: 248 - protein, 112 aa, Artificial Sequence
  Synthetic construct
NIMMTQLPSSLAVSAGEKVTMSCKSSQSVLYISNQKNYLAWFQQKPGQSPKLLIYWASTRESGVPDRFTGSGSGTDFTLTISSVQAEDLAVYYCHQYFSSYTFGGGTKLEIK

SEQ ID NO: 249 - protein, 117 aa, Artificial Sequence
  Synthetic construct
DVQLQESGPDLVKPSQSLSLTCTVTGYSITSGDSWHWIRQFPGNKLEWMGYIHYSGSTKYNPSLKSRISITRDTSKNQFFLQLNSVTTEDTATYYCASWFYYAMDYWGQGTSVTVSS

SEQ ID NO: 250 - protein, 108 aa, Artificial Sequence
  Synthetic construct
ENVLTQSPAIMSASPGEKVTMTCRASSSVSSSYLHWFQQKSGASPKLWIYSTSNLASGVPARFSGSGSGTSYSLTISSVEAEDAATYYCQQYSGFPLTFGAGTKLELK

SEQ ID NO: 251 - protein, 119 aa, Artificial Sequence
  Synthetic construct
QVQLKESGPGLVAPSQSLSITCTVSGFSLTSYGVHWVRQTPGKSLGWLGVIWAGGSASYNSALMSRLSITKDNSKSQVFLKMTSLQTDDTAMYYCARDGSRYEGYFDYWGQGTTLTVSS

SEQ ID NO: 252 - protein, 113 aa, Artificial Sequence
  Synthetic construct
DIVMTQSPSSLAMSVGQKVTLSCKSSQSLLNSNNQKNYLAWYQQKPGQSPKLLVYFASTRESGVPDRFIGSGSGTDFILTISSVQAEDLADYFCQQHYSTPLTFGTGTKLELK

SEQ ID NO: 253 - protein, 121 aa, Artificial Sequence
  Synthetic construct
QIQLVQSGPELKKPGETVKISCKASGYTFTNYGMNWVKQAPGKGLKWMGWINTYTGDPTYADDFKGRFAFSLETSASTAYLQINNLKNEDMATYFCARRISTMITTWFAYWGQGTLVTVSA

SEQ ID NO: 254 - protein, 10 aa, Artificial Sequence
  Synthetic construct
  misc_feature (1)..(10): Xaa can be any naturally occurring amino acid
XXXXXXXXXX

SEQ ID NO: 255 - protein, 121 aa, Artificial Sequence; identical to SEQ ID NO: 253
  Synthetic construct

SEQ ID NO: 256 - protein, 107 aa, Artificial Sequence
  Synthetic construct
DIQMTQSPASLSASVGETVTITCRASENIYSYLAWFQQRQGKSPQLLVYNAKTLTEGVPSRFSGSGSGTQFSLKINSLQPEDFGSYYCQHYQVTPYTFGGGTKLEIK

SEQ ID NO: 257 - protein, 121 aa, Artificial Sequence; identical to SEQ ID NO: 253
  Synthetic construct

SEQ ID NO: 258 - protein, 107 aa, Artificial Sequence; identical to SEQ ID NO: 256
  Synthetic construct

SEQ ID NO: 259 - protein, 10 aa, Artificial Sequence
  Synthetic construct
  misc_feature (1)..(10): Xaa can be any naturally occurring amino acid
XXXXXXXXXX

SEQ ID NO: 260 - protein, 112 aa, Artificial Sequence
  Synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGLYYCWQDTHFPQTFGGGTKLEIK

SEQ ID NO: 261 - protein, 121 aa, Artificial Sequence
  Synthetic construct
DVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCASQGLRYYGYAMDYWGQGTSVTVSS

SEQ ID NO: 262 - protein, 107 aa, Artificial Sequence
  Synthetic construct
DILLTQSPAILSVSPGERVSFSCRASQSIGTIIHWYQQRTNGSPRLLIKYASESISGIPSRFSGSGSGTYFTLSINSVESEDIADYYCQQTNSWPLTFGAGTKLELK

SEQ ID NO: 263 - protein, 120 aa, Artificial Sequence
  Synthetic construct
QVQLQQSGAELARPGASVKMSCKASGYTFTSYTMHWVKQRPGQGLEWIGYINPSTGYTNYNQKFRDKATLTADKSSSTAYMQLSSLTSEDSAVYYCARWVITTGKYFDVWGAGTTVTVSS

SEQ ID NO: 264 - protein, 111 aa, Artificial Sequence
  Synthetic construct
DIVLTQSPASLAVSLGQRATISCRASESVDYYGNSFIHWYQQKPGQPPKLLIYRASNLESGIPARFSGSGSRTDFTLTINPVEADDVATYYCQQSNEDPLTFGAGTKLELK

SEQ ID NO: 265 - protein, 121 aa, Artificial Sequence
  Synthetic construct
QVQLQQSGAELMKPGASVKISCKATGYTFSNYWIEWVKQRPGHGLEWIGEILPGSGRTNYNEKFKGKATFTSITPFNTVFRFSGQLSSLTSEDTAVYYCSRQLGVRAMDYWGQGTSVTVSS

SEQ ID NO: 266 - protein, 10 aa, Artificial Sequence
  Synthetic construct
  misc_feature (1)..(10): Xaa can be any naturally occurring amino acid
XXXXXXXXXX

SEQ ID NO: 267 - protein, 121 aa, Artificial Sequence; identical to SEQ ID NO: 243
  Synthetic construct

SEQ ID NO: 268 - protein, 112 aa, Artificial Sequence
  Synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 269 - protein, 118 aa, Artificial Sequence
  Synthetic construct
EVQLVESGGGLVKPGGSLKLSCAASGFAFSSYDMSWVRQTPEKRLEWVAYISKGGGSTYYPETVKGRFTISRDNAKNTLYLQMSSLKSEDTAMYYCVRHAGNYAMDYWGQGTSVTVSS

SEQ ID NO: 270 - protein, 107 aa, Artificial Sequence
  Synthetic construct
DIKMTQTPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIR

SEQ ID NO: 271 - protein, 123 aa, Artificial Sequence
  Synthetic construct
EVQLQQSGPELVKTGASVKISCKASGYSFTGYYMHWVKQSHGKSLEWIGYINCYNGAINYNQKFKGKATFTVDTSSSTVYMQFNSLTSEDSAVYYCVRAGIHYDYDEAWFAYWGQGTLVTVSA

SEQ ID NO: 272 - protein, 109 aa, Artificial Sequence
  Synthetic construct
QAVVTQESALTTSPGETVTLTCRSSTGPVIIRNYANWVQEKPDHLFTGLIGGTNNRAPGVPARFSGSLIGDKAALTITGAQTEDEAIYFCALWYNNHWVFGGGTKLTVL

SEQ ID NO: 273 - protein, 377 aa, Artificial Sequence
  Synthetic construct
NHVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGGSGGGSKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 274 - protein, 692 aa, Artificial Sequence
  Synthetic construct
PKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGGSGGGSKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 275 - protein, 394 aa, Artificial Sequence
  Synthetic construct
GQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTSY

SEQ ID NO: 276 - protein, 141 aa, Homo sapiens
VCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 277 - DNA, 1071 bp, Artificial Sequence
  Synthetic construct
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 278 - DNA, 1071 bp, Artificial Sequence
  Synthetic construct
tacgaagaggaccactgttcggaagacgagacactcaatggtgtgggtcgtaaggaggactagggtgcgtttcacacattgccttatccataaccacttaaatttctgagtgagaggtatttacgatgcttataatttgtgaagtttttgacgtggaggtagtcaccgctagaggtgtaggacggccaccgtaaatccccactgaggaagtgtgtatgaggaggagacctaggtgtccttgacctataagacttttggcatttcctttagtgtcccaaaaacgactaagtccgaaccggacttttgtcctgcctggaggtacggaaactcttggatctttagtatgcgccgtcctggttcgttgtaccagtcaaaagagaacgtcagcagtcggacttgtattgtaggaaccctaatgcgagggagttcctctattcactacctctacactattaaagtcctttgtttttaaacacgatacgtttatgttatttgaccttttttgacaaaccctggaggccagtcttttggttttaatattcgttgtctccacttttgtcgacgttccggtgtccggtccagacggtacggaacacgagggggctcccgacgaccccgggcctcgggtccctgacgcagagaacggccttacagtcggctccgtcccttacgcacctgttcacgttggaagacctcccactcggttccctcaaacacctcttgagactcacgtatgtcacggtgggtctcacggacggagtccggtacttgtagtggacgtgtcctgcccctggtctgttgacataggtcacacgggtgatgtaactgccgggggtgacgcagttctggacgggccgtcctcagtaccctcttttgttgtgggaccagaccttcatgcgtctgcggccggtacacacggtggacacggtaggtttgacgtggatgcctacgtgacccggtccagaacttccgacaggttgcttacccggattctagggcaggtagcggtgaccctaccacccccgggaggagaacgacgaccaccaccgggacccctagccggagaagtac

SEQ ID NO: 279 - protein, 357 aa, Artificial Sequence
  Synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 280 - DNA, 3186 bp, Artificial Sequence
  Synthetic construct
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgagagcaagtacggccctccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctaccggtccggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 281 - DNA, 3186 bp, Artificial Sequence
  Synthetic construct
tacgacgacgaccactggtcggacgacgacacgctcgacggggtggggcggaaagacgactaggggctgtaggtctactgggtctggtggaggtcggactcgcggtcggacccgctggcccactggtagtcgacggcccggtcggtcctgtagtcgttcatggacttgaccatagtcgtcttcgggctgccgtggcagttcgacgactagatggtgtggtcggccgacgtgtcgccgcacgggtcggccaaatcgccgtcgccgaggccgtggctgatgtcggactggtagaggttggaccttgtccttctatagcggtggatgaaaacggtcgtcccgttgtgtgacgggatgtggaaaccgccgccttgtttcgacctttagtggccgtcgtggaggccgtcgccgttcggaccgtcgccgctcccgtcgtggttcccgctccacttcgacgtcctttcgccgggaccggaccaccgggggtcggtctcggactcgcactggacgtggcactcgccgcactcggacgggctgatgccgcactcgacctaggccgtcggggggtccttcccggaccttaccgacccgcactagaccccgtcgctctggtggatgatgttgtcgcgggacttctcggccgactggtagtagttcctgttgtcgttctcggtccacaaggacttctacttgtcggacgtctggctgctgtggcggtagatgatgacgcggttcgtgatgatgatgccgccgtcgatgcggtacctgatgaccccggtcccgtggtcgcactggcactcgtcgctctcgttcatgccgggagggacggggggaacgggacgggggctcaaggacccgcctgggtcgcacaaggacaagggggggttcgggttcctgtgggactactagtcggcctgggggctccactggacgcaccaccacctgcactcggtccttctagggctccaggtcaagttaaccatgcacctgccgcaccttcacgtgttgcggttctggttcgggtctctccttgtcaagttgtcgtggatggcccaccacagacacgactggcacgacgtggtcctgaccgacttgccgtttcttatgttcacgttccacaggttgttcccggacgggtcgtcgtagcttttctggtagtcgttccggttcccggtcggagcgctcggggtccacatgtgggacggagggagggtccttctctactggttcttggtccacagggactggacggaccacttcccgaagatggggtcgctgtagcggcacctcaccctctcgttgccggtcggactcttgttgatgttctggtggggagggcacgacctgtcgctgccgtcgaagaaggacatgtcggccgactggcacctgttctcggccaccgtccttccgttgcagaaatcgacgtcgcactacgtgctccgggacgtgttggtgatgtgggtcttctcggactcggacagggacccgttctacaagacccacgaccaccaccacccgccccacgaccggacgatgtcggacgaccactgtcaccggaagtagtagaaaacccacgcctcgttcgcctcgtctccgccggtgtcgctgatgtacttgtactgggggtctgccggaccggggtgggccttcgtgatggtcgggatgcggggtgggtccctgaaacggcggatggccaggccgcctcccgcccacttcaagtcgtcttcgcggctgcggggacggatggtcgtcccggtcttagtcgacatgttgctcgacttggacccgtcttcccttctcatgctgcaggacctattcgcctctccggccctgggactctacccgccgttcggagccgccttcttgggggtccttccggacatattgcttgacgtctttctgttctaccggctccggatgtcgctctagccgtacttcccgctcgcctccgccccgttcccggtgctgccggacatagtcccggacaggtggcggtggttcctatggatgctgcgggacgtgtacgtccgggacgggggttccgagctcccgccgcctctcccgtctccttcagaagattgtacgccactgcacctcctcttagggccgggatcctacgaagaggaccactgttcggaagacgagacactcaatggtgtgggtcgtaaggaggactagggtgcgtttcacacattgccttatccataaccacttaaatttctgagtgagaggtatttacgatgcttataatttgtgaagtttttgacgtggaggtagtcaccgctagaggtgtaggacggccaccgtaaatccccactgaggaagtgtgtatgaggaggagacctaggtgtccttgacctataagacttttggcatttcctttagtgtcccaaaaacgactaagtccgaaccggacttttgtcctgcctggaggtacggaaactcttggatctttagtatgcgccgtcctggttcgttgtaccagtcaaaagagaacgtcagcagtcggacttgtattgtaggaaccctaatgcgagggagttcctctattcactacctctacactattaaagtcctttgtttttaaacacgatacgtttatgttatttgaccttttttgacaaaccctggaggccagtcttttggttttaatattcgttgtctccacttttgtcgacgttccggtgtccggtccagacggtacggaacacgagggggctcccgacgaccccgggcctcgggtccctgacgcagagaacggccttacagtcggctccgtcccttacgcacctgttcacgttggaagacctcccactcggttccctcaaacacctcttgagactcacgtatgtcacggtgggtctcacggacggagtccggtacttgtagtggacgtgtcctgcccctggtctgttgacataggtcacacgggtgatgtaactgccgggggtgacgcagttctggacgggccgtcctcagtaccctcttttgttgtgggaccagaccttcatgcgtctgcggccggtacacacggtggacacggtaggtttgacgtggatgcctacgtgacccggtccagaacttccgacaggttgcttacccggattctagggcaggtagcggtgaccctaccacccccgggaggagaacgacgaccaccaccgggacccctagccggagaagtacact

SEQ ID NO: 282 - protein, 1061 aa, Artificial Sequence [sequence omitted]

SEQ ID NO: 283 - protein, 23 aa, Artificial Sequence
  Synthetic construct
IATGMVGALLLLLVVALGIGLFM

SEQ ID NO: 284 - protein, 22 aa, Artificial Sequence
  Synthetic construct
MLLLVTSLLLCELPHPAFLLIP

SEQ ID NO: 285 - protein, 164 aa, Artificial Sequence
  Synthetic construct
VCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2025/0009795 A1
(43) Pub. Date: Jan. 9, 2025

(54) TRUNCATED DOMAIN IV EGFR AND USES THEREOF

(71) Applicant: CITY OF HOPE, Duarte, CA (US)

(72) Inventors: Alfur HUNG, Duarte, CA (US); Miso PARK, Duarte, CA (US); Yi-Chiu KUO, Duarte, CA (US); John C. WILLIAMS, Duarte, CA (US); Sangeeta Bardhan COOK, Duarte, CA (US)

(21) Appl. No.: 18/277,367

(22) PCT Filed: Jan. 14, 2022
(86) PCT No.: PCT/US22/01264

§ 371 Date: Aug. 15, 2023

Related U.S. Application Data
(60) Provisional application No. 63/150,064, filed on Feb. 16, 2021.

(51) Int. Cl. A61K 35/17; A61K 39/00; C07K 14/71; C07K 14/715; C12N 5/0783; C12N 15/86; G01N 33/569

(52) U.S. Cl. CPC A61K 35/17; A61K 39/4611; A61K 39/4631; A61K 39/464404; A61K 39/464412; A61K 39/464419; C07K 14/71; C07K 14/7155; C12N 5/0636; C12N 15/86; G01N 33/56972; A61K 2239/13; A61K 2239/21; A61K 2239/22; C12N 2740/10043; G01N 2333/71

(57) ABSTRACT

A truncated EGFR (tEGFR) cell surface molecule and its uses is provided herein. The tEGFR cell surface molecule includes an EGFR domain IV and does not include an EGFR domain III and may be used, inter alia, as an in vivo tracking marker for genetically modified human T cells. Furthermore, the tEGFR cell surface molecule has cellular depletion potential through mediated through specific anti-domain IV EGFR antibodies. Thus, the tEGFR cell surface molecules provided herein may, inter alia, be used as a non-immunogenic selection tool, tracking marker, a depletion tool or a suicide gene for genetically modified cells having therapeutic potential.

### CROSS-REFERENCE TO RELATED APPLICATIONS

This application claims priority to U.S. Provisional Application No. 63/150,064, filed Feb. 16, 2021, and PCT Application No. PCT/US22/12621, filed Jan. 14, 2022, which are hereby incorporated by reference in their entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-797002WO_ST25.TXT, created on Feb. 16, 2022, 148,094 bytes, machine format IBM-PC, MS Windows operating system, is hereby incorporated by reference.

### BACKGROUND

Immune cell products with homogenous expression of tumor targeting chimeric antigen receptors (CARs) are desirable for clinical evaluation of adoptive therapy strategies to eliminate the product-to-product variability of transgene expression otherwise intrinsic to transduction and other genetic modification procedures without subsequent selection. Immunotherapy using genetically redirected immune cells is an attractive approach for treating minimal residual disease in a variety of cancer patients. However, immunologic rejection of cell products expressing antibiotic selection proteins as part of the transduction strategy has impeded this strategy. A novel selection marker that is not expressed on human lymphocytes, does not contain endogenous signaling or trafficking function, and is recognized by a known, preferably commercially available, pharmaceutical grade antibody reagent that can be utilized for selection, in vivo tracking, and depletion of transduced cells would be a significant improvement in the art. Disclosed herein, inter alia, are solutions to these and other problems in the art.

### BRIEF SUMMARY OF THE INVENTION

Compositions and methods for purification, both in vivo and ex vivo, of genetically modified cells are provided herein. The genetically modified cells may be modified by transduction, or any other process that adds, deletes, alters, or disrupts an endogenous nucleotide sequence. The genetically modified cells may be transduced T cells with altered activity, including altered immunoactivity.

Provided herein is a non-immunogenic selection epitope compatible with, for example, immunomagnetic selection, which facilitates immunotherapy in cancer patients without undesirable immunologic rejection of cell products (i.e. as seen when expressing antibiotic selection proteins). In some embodiments, the non-immunogenic selection epitope is an endogenous cell-surface molecule that is modified or truncated to retain an extracellular epitope recognized by an antibody or functional fragment thereof (e.g., the anti-domain IV antibody provided herein), and to remove any signaling or trafficking domains and/or any extracellular domains unrecognized by the antibody (e.g., the anti-domain IV antibody provided herein). The removal of the signaling or trafficking domains and/or any extracellular domains unrecognized by the antibody (e.g., the anti-domain IV antibody provided herein) renders the endogenous cell-surface molecule inert, which is a desired property for the molecule, while being anchored in the cell membrane through a transmembrane domain. The non-immunogenic selection epitope may also be used as a selection tool or tracking marker.

The modified endogenous cell-surface molecule may be, but is not limited to, any cell-surface related receptor, ligand, glycoprotein, cell adhesion molecule, antigen, integrin or cluster of differentiation (CD) that is modified as described herein. In embodiments, the modified endogenous cell-surface molecule is a truncated tyrosine kinase receptor. In one aspect, the truncated tyrosine kinase receptor is a member of the epidermal growth factor receptor family (e.g., ErbB1, ErbB2, ErbB3, ErbB4).

Epidermal growth factor receptor, also known as EGFR, ErbB1 and HER1, is a cell-surface receptor of the epidermal growth factor family of extracellular ligands. Alterations in EGFR activity have been implicated in certain cancers. In embodiments, a gene encoding an EGFR polypeptide is provided that is formed by removal of nucleic acid sequences that encode polypeptides including the membrane distal EGF-binding domain and the cytoplasmic signaling tail (a “truncated EGFR”, “tEGFR” or “EGFRt”), but retains the extracellular domain IV epitope recognized by any anti-EGFR antibody (e.g., anti-domain IV EGFR antibody) provided herein including embodiments thereof. In embodiments, tEGFR does not include EGFR domain III. In embodiments, tEGFR includes a transmembrane domain. In embodiments, the transmembrane domain is a EGFR transmembrane domain.

Application of biotinylated-anti-EGFR antibodies (e.g., anti-domain IV EGFR antibodies), provided herein to immunomagnetic selection in combination with anti-biotin microbeads successfully will enrich T cells that are lentivirally transduced with EGFRt-containing constructs from as low as 2% of the population to greater than 90% purity without observable toxicity to the cell preparation. Constitutive expression of this inert EGFRt cell surface molecule does not affect T cell phenotype or effector function as directed by the coordinately expressed chimeric antigen receptor (CAR), CD19R. Through flow cytometric analysis, EGFRt will be successfully utilized as an in vivo tracking marker for T cell engraftment in mice. Furthermore, EGFRt will be shown to have suicide gene potential through anti-EGFR antibodies (e.g., anti-domain IV EGFR antibodies)-mediated antibody dependent cellular cytotoxicity (ADCC) pathways. Thus, EGFRt may be used as a non-immunogenic selection tool, tracking marker, and suicide gene for transduced T cells that have immunotherapeutic potential. The EGFRt nucleic acid may also be detected by means well known in the art.

In another embodiment, methods of discovering and designing modified, truncated or altered endogenous cell-surface molecules which bind to anti-EGFR antibodies (e.g., anti-domain IV EGFR antibodies) as described herein are provided. The methods include modeling the protein of interest and truncating functional portions, while leaving the antibody-binding portions intact. The resulting modified receptor or ligand can be sorted using a labeled antibody and then enriched such that the concentration of the modified receptor or ligand is increased.

Yet another embodiment provides a method of selecting a transduced T cell including transducing a population of T cells with a modified, truncated or altered endogenous cell-surface molecule gene sequence (e.g., truncated EGFR) and then contacting an antibody that binds the modified ligand or receptor sequence to the transduced T cells. If the modified receptor sequence is EGFRt, the antibody is preferably a biotinylated anti-EGFR antibody (e.g., anti-domain IV EGFR antibody). The T cells are then sorted by adding anti-biotin microbeads and selected using immunomagnetic separation, adding fluorochrome-conjugated anti-biotin and selecting the T cells using Fluorescence Activated Cell Sorting, or any other reliable method of sorting the cells. The modified ligand or receptor sequences, such as the EGFRt sequence, may be contained in a suitable transfer vehicle such as a lentiviral vector.

These and other embodiments are further explained in the drawing and detailed description herein.

### BRIEF DESCRIPTION OF THE DRAWINGS

FIG. 1. is a schematic representation of the EGFR protein domains. Mutations within the extracellular domains (ECD) are shown, several of which are shown to induce EGFR auto-phosphorylation, which in turn promotes cellular transformation.

FIG. 2. shows the superposition of structures of therapeutic anti-EGFR antibodies in complex with domain III of EGFR. The binding location of the antibodies is consistent with inhibition of EGF binding to EGFR. The anti-EGFR antibody Fabs are shown as ribbon diagrams and EGFR is shown as a surface rendering. The PDB accession codes for the overlaid structures are as labeled.

FIG. 3. shows non-antibody EGFR-binding therapeutics in complex with EGFR domain III. The non-antibody EGFR-binding therapeutics are depicted as ribbon diagrams and EGFR is shown as a surface rendering. The PDB accession codes for the structures are as labeled.

FIG. 4. shows the superposition of various Her2 targeting biologics with Her2. The binding of trastuzumab to the juxtamembrane domain (domain IV) of Her2 is labeled. Her2 is represented as a surface rendering and Her2 targeting biologics are shown as ribbon diagrams.

FIGS. 5A-5C. show the correlation between T cell activation and the proximity of the epitope and cell membrane. FIG. 5A, illustrates the distance between the cancer cell membrane and T cell membrane upon formation of the T cell receptor (TCR)/CD3 complex involved in antibody-dependent cellular cytotoxicity (ADCC) activity. FIG. 5B. demonstrates that the geometry of the Her2 antigen in respect to the cancer cell membrane is critical to achieve effective ADCC activity. Targeting of domains closer to the cancer cell membrane results in greater ADCC effect while targeting the domain furthest from the membrane does not result in effective ADCC activity. FIG. 5C. is a graph illustrating the results from a T cell activation assay, showing that targeting of antigen domains closer to the cell membrane effectively produces high ADCC activity compared to antigen domains further from the membrane.

FIG. 6. are schematics showing the constructs of chimeric human EGFR domain IV proteins D4-IgG2a, EGFR-D4-IgG2a and EGFR-IGA1.

FIG. 7. are chromatograms depicting purification of mouse D4-IgG2A (top panel) and human EGFR-D4-IgA1 (bottom panel) with size exclusion chromatography. The chromatography indicates the antibodies were purified to substantial homogeneity.

FIG. 8. illustrates results from an ELISA experiment identifying six hybridoma clones that bind human EGFR-IgA protein. EGFR-IgA proteins were immobilized in microwells and were subsequently incubated with hybridoma culture supernatant. Results indicate that the 5C8 clone had the highest antibody titer.

FIG. 9. shows results from a flow cytometry experiment testing binding of hybridoma clones to SKOV3 cells. Results indicate that the 5C8 antibody clone binds to the ovarian cancer cell line SKOV3 compared to an isotype control.

FIG. 10. shows results from an ELISA experiment illustrating the specificity of the 5C8 clone for binding with various recombinant proteins. The results indicate that the clone is specific for binding to D4-IgA protein which includes EGFR domain IV fused with human IgA1.Fc, and EGFR-His.

FIG. 11. illustrates results from a flow cytometry experiment showing that the 5C8 clone is specific for EGFR-expressing cells, for example the breast cancer cell line SKBR3. As expected, the 5C8 antibody does not bind to Jurkat cells which do not express EGFR.

FIG. 12. illustrates results from an ELISA experiment showing that the Ig isotypes of the 5C8 clone are γ1 and λ.

FIGS. 13A and 13B. show purification and characterization of the anti-domain IV EGFR antibody clone EGFRD4-5C8. FIG. 13A. is a chromatogram depicting purification of EGFRD4-5C8 using size exclusion chromatography and FIG. 13B. shows a reducing (R) and non-reducing (NR) gel characterizing the purified product. As expected, the non-reducing gel showed a product at approximately 150 kDa while the reducing gel showed two bands at approximately 25 kDa and 50 kDa.

FIGS. 14A-14C. are sensorgrams from SPR experiments that depict binding of FIG. 14A. EGFR-D4-5C8 antibody, FIG. 14B. cetuximab Fab, and FIG. 14C. wild type traszumab to EGFR domain IV. EGFR-D4-5C8 was shown to be captured by immobilized EGFR domain IV, while cetuximab Fab and wild type trastuzumab do not show substantial binding to domain IV of EGFR.

FIGS. 15A-15C. are sensorgrams from SPR experiments showing binding of FIG. 15A. EGFR-D4-5C8 Fab, FIG. 15B. cetuximab Fab, and FIG. 15C. wild type trastuzumab Fab to EGFR domain IV. As expected, EGFR-D4-5C8 Fab was shown to be captured by immobilized EGFR domain IV, while cetuximab Fab and wild type trastuzumab Fab do not show substantial binding to domain IV of EGFR.

FIG. 16. are representative images of Western blots showing detection of phosphorylated-EGFR (phsopho-EGFR), phosphorylated Akt (phospho-Akt), or β-actin (positive control) upon incubating the human ovarian carcinoma cell line OVCAR3 with no antibody, EGF, cetuximab or the EGFR-D4-5C8 antibody clone. Upon addition of cetuximab, no phosphor-EGFR was detected, as expected. However, phospho-EGFR was detected upon addition of EGFR-D4-5C8. The results indicate that cetuximab and EGFR-D4-5C8 affect different cellular pathways.

FIG. 17. shows flow cytometry data detecting the binding of cetuximab (top row) or EGFR-D4-5C8 (bottom row) to MDA-MB-468, SCOV3 or OVCAR3 cells. The similar binding abilities of cetuximab and EGFR-D4-5C8 to the cells may be attributed to the fluorophore intensity and binding affinity of the secondary antibody PE anti-Fc.

FIG. 18A. show results from an ADCC assay. MDA-MB-468 cells and SKOV3 cells were incubated with Jurkat cells expressing NFAT-regulated luciferase and either 5C8 or cetuximab antibodies at different concentrations. Luciferase substrate was added to each solution, and ADCC activity was measured based on luminescence intensity.

FIG. 18B. is flow cytometry data showing that cetuximab, anti-HER3 antibody and a dual anti-EGFR/HER3 meditope-enabled antibody bind to the bladder carcinoma cell line 486 and the ovarian cancer cell line SKOV3 (left panel); the EGFR-D4-5C8 antibody also shows binding ability to both cell lines (right panel).

FIGS. 19A-19G. are results from ADCC assays conducted with Jurkat cells and various EGFR-expressing cancer cell lines incubated with the 5C8 antibody clone or cetuximab. FIG. 19A. MDA-MB-468 cells, FIG. 19B. SW48 cells, FIG. 19C. SKOV3 cells, FIG. 19D. A549 cells, and FIG. 19E. HCT116 cells were incubated with 5C8 or cetuximab antibody at different concentrations. After a 6 hr incubation, luciferase substrate was added in each well to react with luciferase expressed by Jurkat cells. ADCC activity was measured based on luminescence intensity. Results from the assays show that the 5C8 antibody exerts stronger ADCC effects than cetuximab. FIG. 19F. shows the combined ADCC for the 5C8 clone and FIG. 19G. shows the combined ADCC for cetuximab.

FIG. 20. shows binding of EGFR-targeting antibodies to EGFR-expressing cancer cell lines, as determined by flow cytometry. Cells were incubated with 10 μg/ml 5C8 or 10 μg/ml cetuximab for 30 minutes, and subsequently washed, followed by staining of cells with anti-kappa-Alexa-647 secondary antibody. Histogram analysis (top panel) and median fluorescence intensity of each cell line before and after antibody binding to the cells (bottom panel) is depicted.

FIG. 21. is the time course for in vivo ADCC experiments using animal xenograft models showing. EGFR⁺ tumor killing. For the experiment, female SCID mice were subcutaneously injected with five million MDA-MB-468 cells on day 1. Starting on day 7, 5 mg/kg of 5C8-IgG1, 5C8-IgG2a, or cetuximab-IgG2a antibodies were intraperitoneally injected into the mice two times per week. A total nine doses were administered to the mice. Tumor volume was calculated as V=(L×W×W)/2, following caliper measurement to determine the length and width of the tumors.

FIG. 22. illustrates results from an ADCC experiment using 5C8-IgG1, 5C8-IgG2a, or cetuximab-IgG2a antibodies. Results indicate the 5C8-IgG2a antibody can eradicate tumors in vivo, while the 5C8-IgG1 antibody does not have a therapeutic effect as compared with the vehicle control.

FIG. 23. is a phylogram showing sequence distance relationships between the identified anti-domain IV EGFR antibody clones.

FIG. 24. Depicted are phylograms comparing the sequence relationships between the identified anti-domain IV EGFR antibody clones and the sequence alignment of various antibody clones.

FIGS. 25A-25F. are chromatograms showing purification of anti-domain IV EGFR antibody clones FIG. 25A. EGFRD4-7Ab, FIG. 25B. EGFRD4-28Ab, FIG. 25C. EGFRD4-30Ab, FIG. 25D. EGFRD4-31Ab, and FIG. 25E. EGFRD4-34Ab by size exclusion chromatography; and FIG. 25F. a representative image of a non-reduced and reduced gel showing separation of the purified products. As expected, a higher molecular weight band for each sample is shown on the non-reducing gel, while the reducing gel shows two bands at approximately 50 kDa and 25 kDa for each antibody sample.

FIGS. 26A-26F. show sensorgrams from SPR experiments depicting binding of various anti-domain IV EGFR antibodies to domain IV of EGFR. EGFR domain IV was immobilized on an CM5 chip at a density of 500 RU through EDC/NHS coupling. The EGFRD4 (IgG) antibodies FIG. 26A. EGFRD4-7Ab, FIG. 26B. EGFRD4-28Ab, FIG. 26C. EGFRD4-30Ab, FIG. 26D. EGFRD4-31Ab, FIG. 26E. EGFRD4-34Ab, and FIG. 26F. EGFRD4-5C8 were prepared in HBS-EP⁺ running buffer and were injected at concentrations of 30 nM, 10 nM, and 3 nM at 25° C.

FIG. 27. are sensorgrams from SPR experiments showing binding of the EGFRD4-5C8 clone to EGFR domain IV. EGFR domain IV was immobilized on an CM5 chip at a density of 500 RU through EDC/NHS coupling, and various concentrations of EGFRD4-5C8 were prepared in HBS-EP⁺ running buffer and injected. SPR binding curves for 30 nM, 10 nM, 3 nM and 1 nM EGFRD4-5C8 are shown (top panel), in addition to a magnified portion of the sensorgram showing binding curves for 10 nM, 3 nM and 1 nM EGFRD4-5C8 (bottom panel).

FIG. 28A illustrates the selection of EGFRt+ T cells using biotinylated an anti-EGFR antibody (e.g., anti-domain IV EGFR antibody) as provided herein. A schematic is shown of the EGFR antibody biotinylation and reformulation process.

FIG. 28B depicts schematics of both the immunomagnetic (top) and the fluorescence activated cell sorting (bottom) EGFRt selection procedures.

FIG. 28C depicts schematics of the CD19CAR-T2A-EGFRt (left) and CD19CAR-T2A-EGFRt-IMPDH2dm (right) constructs contained in lentiviral vectors used for the immunomagnetic selection of various T cell lines lentivirally transduced with CAR and EGFRt containing constructs. Codon optimized sequence portions of the CD19-specific, CD28 co-stimulatory CAR, followed by the self-cleavable T2A, EGFRt and IMPDH2dm selection markers are indicated, along with the Elongation Factor 1 promoter sequences (EF-1p), and the GCSFR alpha chain signal sequences (GCSFRss, which directs surface expression).

FIG. 29 is the nucleotide (sense strand is SEQ ID NO:277, antisense strand is SEQ ID NO:278) sequence and amino acid (SEQ ID NO:279) sequences of GMCSFR alpha chain signal sequence linked to EGFRt including domain III and domain IV. The GMCSFR alpha chain signal sequence, which directs surface expression, is encoded by nucleotides 1-66. EGFRt including domain III and IV is encoded by nucleotides 67-1071.

FIG. 30 is the nucleotide (sense strand is SEQ ID NO:280, antisense strand is SEQ ID NO:281) sequence and amino acid (SEQ ID NO:282) sequences of CD19R-CD28gg-Zeta (CO)-T2A-EGFRt. CD19R-CD28gg-Zeta (CO) is encoded by nucleotides 1-2040; T2A is encoded by nucleotides 2041-2112; GMCSFR is encoded by nucleotides 2113-2178; EGFRt including domain III and domain IV is encoded by nucleotides 2179-3186.

FIGS. 31A and 31B. illustrate FIG. 31A. data showing thermal stability curves of EGFR D4 5C8Fab, EGFR D4 28Fab, EGFR D4 30Fab, EGFR D4 31Fab, EGFR D4 34 Fab and meTras 183E Fab (meditope-enabled trastuzumab), and FIG. 31B. the melting point (Tₘ) of each Fab domain. Each protein in this study is a murine-human chimeric Fab.

FIG. 32. shows binding of EGFR-targeting antibodies to EGFR-expressing cancer cell lines MDA-MB-468. SKOV3 and OVCAR-3, as determined by flow cytometry. Because the 5C8 antibody has lower binding affinity, more antibody is needed to detect a shift in intensity.

FIGS. 33A-33C. shows binding of EGFR D4 targeting antibodies and cetuximab to FIG. 33A. MDA-MB-468. MDA-MB-468. FIG. 33B. SKOV3, OVCAR-3, FIG. 33C. U87, and U87-EGFRviii cells, as determined by flow cytometry. The cells were detached by trypsin and incubated with 1, 10, or 100 μg/ml EGFR Ab in 2% BSA in PBS at 4° C. After 30 minutes, cells were washed with 2% BSA in PBS. The cells were then stained with secondary antibodies, either anti-Fc PE for MDA-MB-468, MDA-MB-231, SKOV3 and OVCAR3 cells, or anti-Fc Alexa-647 for U87 and U87-EGFRviii cells at 4° C. for 30 min. The cells were analyzed by flow cytometry after washing.

FIGS. 34A-34D. illustrates results from cell viability assays conducted with cetuximab, EGFR-D4 targeting antibody, or a combination thereof and cancer cell lines FIG. 34A. MDA-MB-468, FIG. 34B. SKOV3 and FIG. 34C. OVCAR-3. Cells were incubated with different concentrations of the indicated Ab for 72 h and cell viability was assessed using the Promega CellTiter-Glo® Luminescence kit based on the manufacture's instructions. Cetuximab was selected to intentionally block EGF from binding to EGFR by Mendelsohn (PMID: 6298788) and Rodeck (PMID: 2250044). The reasoning was that cancer cells overexpressing EGFR (e.g. MDA-MB-468) are onco-addicted; thus, by blocking EGF from binding to EGFR the cells would undergo apoptosis. This hypothesis works well for EGFR onco-addicted cell lines. For the EGFR D4 antibody, since domain three of EGFR was not targeted, blocking of EGF binding was not expected. Thus, results obtained from this study were as predicted. A feature of Cetuximab, pantitumab and other mAbs that block EGF binding and signaling pathways, is that skin rash is typically produced. Thus, this may be an important distinguishing feature of EGF blocking antibodies versus non-EGF blocking approaches. FIG. 34D. is a schematic showing a mechanism of resistance to domain III targeting therapeutics. Upon EGF binding, the extracellular domain of EGFR switches from an inactive tethered state to an active untethered state. CTX selection, which prevents EGF binding by interacting with domain III of tethered EGFR, can lead to cells acquiring EGFR mutations (G33S, N56K) in domain I, which leads to ligand-independent activation and prevents receptor internalization/degradation. Mutant EGFR does not bind CTX since it is ‘trapped’ in the open confirmation, leading to CTX resistance (PLOS One. 2020 Feb. 18; 15 (2): e0229077).

FIGS. 35A and 35B. illustrate results from ADCC assays with Jurkat cells expressing immunogloulin receptor CD16-158F and FIG. 35A. MDA-MB-468 cells and FIG. 35B. SKOV3 cells. ADCC predominantly works through the FcRIIIa receptor expressed on NK cells. Tumor cells (2.5e4) were seeded in a 96-well white wall plate on day 1 and allowed to attach overnight. On day 2, medium was removed from the plate and Jurkat cells (1e5) expressing CD16-158F were added in each well with Ab at the indicated concentration. The final volume was 60 ul per well. After incubation for 6 h, 50 ul of luciferase substrate was added in each well and luminescence was measured immediately. There is a polymophormism in humans at residue 158 of CD16, which is either a valine or phenylalanine (PMID: 10444269). The valine substitution is binds more strongly to human IgG1 and thus produces a stronger ADCC effect. This study tests the 158F variant. Results show that ADCC is observed for the 5C8 clone but not for two other EGFR targeting mAbs, cetuximab and Duligotuzumab (2n1). CD16-158F has low affinity to Fc, and thus, weak ADCC activity was shown.

FIGS. 36A-36E. illustrates binding of EGFR-targeting antibodies to EGFR expressing cancer cell lines and ADCC activity through Jurkat cells expressing immunoglobulin receptor CD16-158V. FIG. 36A. shows results from FACS studies assessing binding of EGFR-targeting antibodies to cancer cells, and FIG. 36B. shows MFI indicative of antibody binding. Cells were detached by trypsin and incubated with 10 μg/ml cetuximab or 5C8 in 2% BSA in PBS at 4° C. After 30 min, cells were washed by 2% BSA in PBS followed by staining cells with anti-Fc Alexa-647 secondary antibody at 4° C. for 30 min. Cells were analyzed by flow cytometry after wash. Jurkat activation assay results are illustrated in studies using FIG. 36C. EGFR D4 5C8 antibody and FIG. 36D. Cetuximab. Tumor cells (2.5e4) were seeded in a 96 well white wall plate on day 1 and allowed to attach overnight. On day 2, medium was removed from the plate and Jurkat cells (1e5) expressing CD16-158V were added to each well with Ab at the indicated concentration. The final volume was 60 ul per well. After incubation for 6 h, 50 ul of luciferase substrate was added in each well and luminescence was measured immediately. As noted in the FIGS. 32A and 32B, CD16 polymorphism can alter ADCC activity. The CD16-158V Jurkat cells were generated to access any differences in ADCC. Based on the data, the level of ADCC is much higher using CD16 with this polymorphism (as compared to the 158F polymorphism). Critically, y-axes of each graph are different. The cetuximab panel (FIG. 33D) is ˜4× expanded compared to the 5C8 panel (FIG. 33C). In other words, cetuximab can activate ADCC but it is far less effective compared to the 5C8 mAb. FIG. 36E. illustrates FACS data showing CD16-158V expression on Jurkat cells used in this study.

FIGS. 37A-37E. shows results of ADCC assays through Jurkat cells expressing CD16-158V and FIG. 37A. MDA-MB-468, FIG. 37B. SCOV3, FIG. 37C. SW48, FIG. 37D. A549 and FIG. 37E. HCT-116 cells. The data show a direct comparison of ADCC using EGFR D4-targeting antibody and Cetuximab, and show that the EGFR D4 targeting antibody is significantly more effective than Cetuximab. Tumor cells (2.5e4) were seeded in 96 well white wall plate on day 1 and allowed to attach overnight. On day 2, medium was removed from the plate and Jurkat cells (1e5) with CD16-158V expression were added in each well with Ab at the indicated concentration at the final volume of 60 ul per well. After incubation for 6 h, 50 ul of luciferase substrate was added in each well and luminescence was measured immediately.

FIGS. 38A-38C. illustrate EGFR binding to triple-negative breast cancer (TNBC) cell lines and ADCC activity. FIG. 38A. shows FACS data assessing various concentrations of indicated antibody binding to MDA-MB-468 and MDA-MB-231 cells. Cells were detached by trypsin and incubated with 1, 10, or 100 μg/ml EGFR Ab in 2% BSA in PBS at 4° C. After 30 min, the cells were washed by 2% BSA in PBS, followed by staining with anti-Fc PE secondary antibody at 4° C. for 30 min. Cells were analyzed by flow cytometry after after washing. FIG. 38B. illustrates ADCC with using Jurkat cells expressing low affinity CD16-158F and FIG. 38C. illustrates ADCC activity with Jurkat cells expressing high affinity CD16-158V. ADCC assays were performed by co-culture of CD16-expressing Jurkat cells (1e5) and EGFR-expressing cancer cells (2.5e4) in the presence EGFR Ab at the indicated concentrations. The final incubation volume was 60 ul. After 6 h incubation, 50 ul luciferase substrate was added to each well to react with luciferase expressed by the Jurkat cells. ADCC activity was measured based on luminescence intensity. The results show that CD16-158V overexpressed in Jurkat cells had higher affinity to Fc compared to CD16-158F, and therefore induced higher ADCC activity.

FIGS. 39A-39C. show ADCC activity through high affinity and low affinity CD16 receptors. FIG. 39A. illustrates binding of Cetuximab and EGFR D4 targeting antibodies to MDA-MB-468 cells. Cells were detached by trypsin and incubated with 1, 10, or 100 μg/ml EGFR Ab in 2% BSA in PBS at 4° C. After 30 min, cells were washed by 2% BSA in PBS followed by staining cells with anti-Fc PE secondary antibody at 4° C. for 30 min. Cells were analyzed by flow cytometry after washing. FIG. 39B. shows results from ADCC assays through high affinity CD16-158V and FIG. 39C. illustrates results of ADCC assays through low affinity CD16-158F. ADCC assays were performed by co-culture of CD16-expressing Jurkat cells (1e5) and EGFR-expressing cancer cells (2.5e4) in the presence of EGFR-targeting Ab at different concentrations. The final incubation volume is 60 ul. After 6 h incubation, 50 ul luciferase substrate was added in each well to react with luciferase expressed by Jurkat cells. ADCC activity was measured based on luminescence intensity. The results showed that CD16-158V overexpressed in Jurkat cells had higher affinity to Fc compared to CD16-158F, and therefore induced higher ADCC activity.

FIG. 40. illustrates ADCC results through PBMC. In each well of a microplate, PBMC (2e5) and MDA-MB-468 tumor cells (1e4) were incubated for 12 h with or without the indicated Ab. ET ratio=20:1. The left panel illustrates the percentage of tumor cells remaining after PBMC treatment, with control tumor cells (no treatment) set to 100%. The right panel illustrates the percentage of tumor cells killed by PBMC. PBMC #1, PBMC #2 and PBMC #3 were from different donors. The previous ADCC assays were based on engineered Jurkat cells that quantified ADCC activation through NFAT driven luciferase expression. This method is consistent and efficient. As an alternative, PBMC isolated from patients can be used, and cell killing measured. The left panel shows the number of viable tumor cells after treatment with PBMC. In the absence of PMBC, cetuximab kills a fraction of the cells. Without wishing to be bound by theory, this may be due to EGF blockade and onco-addiction. The presence of the Ab further reduces the number of viable cells, although it is noted that the PBMC alone reduce the number of tumor cells. Thus, while there are differences between the 5C8 clone and the cetuximab, these differences likely reflect the combination of onco-addiction and PMBC killing beyond ADCC activity. The right panel represents the same results, but is represented as percent cell death.

FIGS. 41A-41C. illustrates results from ADCC assays through PBMC with cell lines FIG. 41A. MDA-MB-468. FIG. 41B. U87 and FIG. 41C. U87-EGFRviii. In each well. PBMC (2e5) and tumor cells (1e4) were incubated for 6 h with or without the indicated Ab. Tumor cells without any treatment were set as 100%. The E:T ratio was 20:1. The results illustrate that Cetuximab barely affected cell viability in the absence of PBMCs. Further, the addition of only PBMC dramatically reduced cell viability for all cell lines studies. Results using the U87 line are compelling-incubation with only PBMC drastically reduces viability. Without PBMC, addition of the 5C8 antibody and Cetuximab do not result in significant decreases in cell viability. Additional studies are performed with these and other cell lines to assess conditions including receptor environment, recycling, and other intrinsic factors.

FIGS. 42A-42D. show percent cell killing by PBMC as assessed by ADCC assays for FIG. 42A. MDA-MB-468. FIG. 42B. U87 and FIG. 42C. U87-EGFRviii cells, and FIG. 42D. FACS data showing binding of Cetuximab and EGFR D4 5C8 to the cell lines MDA-MB-468. U87 and U87-EGFRviii. For ADCC assays, in each well. PBMC (2e5) and tumor cells (1e4) were incubated for 6 h with or without indicated Ab. Tumor cells without any treatment were set as 100%. The E:T ratio was 20:1. For FACS analysis, cells were detached by trypsin and incubated with 1, 10, or 100 μg/ml EGFR Ab in 2% BSA in PBS at 4° C. After 30 min, cells were washed by 2% BSA in PBS followed by staining cells with anti-Fc PE (for MDA-MB-468) or anti-Fc Alexa-647 (for U87 and U87-EGFRviii) secondary antibodies at 4° C. for 30 min. Cells were analyzed by flow cytometry after washing.

FIG. 43. shows a treatment plan for animal xenograft models for EGFR⁺ tumor killing to assess in vivo ADCC. Female SCID mice were subcutaneously injected with MDA-MB-468 breast cancer cells at 5×10⁶ cells/site/mouse on day one. Starting on day 7, 5 mg/kg antibodies were intraperitoneally injected into mice twice per week. A total nine doses were given to the mice. A caliper was used to measure length and width of tumor, and tumor volume was calculated as V=(L×W×W)/2. For the study, mouse strains SCID (BALB/c-Igh^{b scid}) were used. The mice were divided into the following treatment groups: PBS, 5 mg/kg, 5 mice; 5C8-IgG1, 5 mg/kg, 5 mice; 5C8-IgG2a, 5 mg/kg, 5 mice; Cetuximab-IgG2a, 5 mg/kg, 5 mice. The Ab were delivered intraperitoneally. The weight of each mouse was ˜20 mg, and antibody was administered at ˜100 mg/mouse. Standard Model Endpoints included tumor volumes (V=(W²×L)/2 for caliper measurements) and Kaplan-Meier survival analysis.

FIG. 44. shows the MDA-MB-468 xenograft-tumor volume of mice receiving various antibody treatments or PBS vehicle control, as indicated. The treatment groups were: PBS, 5 mg/kg, 5 mice; 5C8-IgG1, 5 mg/kg, 5 mice; 5C8-IgG2a, 5 mg/kg, 5 mice; Cetuximab-IgG2a, 5 mg/kg, 5 mice. The Ab were delivered intraperitoneally. The weight of each mouse was ˜20 mg, and antibody was administered at ˜100 mg/mouse.

FIG. 45. shows a treatment plan for animal xenograft models for EGFR⁺ tumor killing to assess in vivo ADCC. Female SCID mice were subcutaneously injected with MDA-MB-468 breast cancer cells at 7×10⁶ cells/site/mouse on day one. Starting on day 8, 5 mg/kg antibodies were intraperitoneally injected into mice twice per week. A total of ten doses were given to each mouse. Tumor volume was calculated as V=(L×W×W)/2 after a caliper was used to measure length and width of tumor. For the study, mouse strains SCID (BALB/c-Igh^{b scid}) were used. The mice were divided into the following treatment groups: PBS, 5 mg/kg, 4 mice; 5C8-IgG1, 5 mg/kg, 4 mice; 5C8-IgG2a, 5 mg/kg, 4 mice. The antibodies were delivered intraperitoneally. The weight of each mouse was ˜20 mg, and antibody was administered at ˜100 mg/mouse. Standard Model Endpoints included tumor volumes (V=(W²×L)/2 for caliper measurements) and Kaplan-Meier survival analysis.

FIG. 46. shows the MDA-MB-468 xenograft-tumor volume of mice receiving various antibody treatments or PBS vehicle control, as indicated. It was noted that the group treated with Cetuximab-IgG2a had abdominal distension. This was possibly due to endotoxin contamination for Cetuximab IgG2a Ab. Thus, the mouse group treated with cetuximab-IgG2a was removed from this data set. It was noted that endotoxin units for Cetuximab IgG2a was 0.112 EU/ug, while endotoxin units for 5C8 IgG1 was 0.016 EU/ug. The tumors were allowed to grow to a large size before initiating the treatment. A murine version of cetuximab was used for this study. However, subsequent testing of the protein sample indicated substantial amounts of endotoxin, as noted above. Replicates of this study are underway.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., EGFR) in other proteins with different numbering systems. For example, by performing a simple sequence alignment with a protein (e.g., EGFR) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the residue to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see. e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see. e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see. e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below; due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see. e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3).

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F (ab)′2, a dimer of Fab which itself is a light chain joined to V_{H}-C_{H1} by a disulfide bond. The F (ab)′2 may be reduced under mild conditions to break the disulfide linkage in the hinge region, thereby converting the F (ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeven et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g, variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “EGFR protein” or “EGFR” as used herein includes any of the recombinant or naturally-occurring forms of epidermal growth factor receptor, also known as Proto-oncogene c-ErbB-1, Receptor tyrosine-protein kinase erbB-1, ERBB, ERBB1, HER1, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids. A vector” may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art. Transcription of the DNA encoding the polypeptides of the present invention by higher eukaryotes is increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cy tomegalovirus early promoter enhancer, the polyoma enhancer on the late side of the replication origin, and adenovirus enhancers.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See. e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego. The term detectable moiety includes a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “cell surface molecule” as used herein, refers to a molecule wherein at least a portion of the molecule is expressed on the surface of a cell. In embodiments, the cell surface molecule spans the membrane of a cell including an extracellular portion and a transmembrane portion.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. EGFR protein). Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. EGFR protein) relative to the activity or function of the protein in the absence of the inhibitor. In embodiments inhibition means negatively affecting (e.g. decreasing) the concentration or levels of EGFR relative to the concentration or level of the protein in the absence of the inhibitor. In embodiments inhibition refers to reduction of a disease or symptoms of disease. In embodiments, inhibition refers to a reduction in the activity of EGFR. Thus, inhibition includes, at least in part, partially or totally blocking stimulation, decreasing, preventing, or delaying activation, or inactivating, desensitizing, or down-regulating signal transduction or enzymatic activity or the amount of EGFR. In embodiments, inhibition refers to a reduction of activity of EGFR resulting from a direct interaction (e.g. an inhibitor binds to EGFR). In embodiments, inhibition refers to a reduction of activity of EGFR from an indirect interaction (e.g. an inhibitor binds to a protein that activates EGFR, thereby preventing target protein activation).

Thus, the terms “inhibitor,” “repressor” or “antagonist” or “downregulator” interchangeably refer to a substance capable of detectably decreasing the expression or activity of a given gene or protein (e.g. EGFR protein). The antagonist can decrease EGFR expression or activity 10%, 20%, 30%, 40%, 50%, 60%, 70%, 80%, 90% or more in comparison to a control in the absence of the antagonist. In certain instances, EGFR expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., breast cancer, lung cancer)) means that the disease (e.g. cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The term “genetic modification” means any process that adds, deletes, alters, or disrupts an endogenous nucleotide sequence and includes, but is not limited to viral mediated gene transfer, liposome mediated transfer, transformation, transfection and transduction, e.g., viral mediated gene transfer such as the use of vectors based on DNA viruses such as lentivirus, adenovirus, retroviruses, adeno-associated virus and herpes virus.

“Variant” refers to polypeptides having amino acid sequences that differ to some extent from a native sequence polypeptide. Ordinarily, amino acid sequence variants will possess at least about 80% sequence identity, more preferably, at least about 90% homologous by sequence. The amino acid sequence variants may possess substitutions, deletions, and/or insertions at certain positions within the reference amino acid sequence.

“Antibody-dependent cell-mediated cytotoxicity” and “ADCC” refer to a cell-mediated reaction in which nonspecific cytotoxic cells that express Fc receptors, such as natural killer cells, neutrophils, and macrophages, recognize bound antibody on a target cell and cause lysis of the target cell. ADCC activity may be assessed using methods, such as those described in U.S. Pat. No. 5,821,337.

“Effector cells” are leukocytes which express one or more effector cell ligands (e.g. constant region receptors such as CD16) and perform effector functions. The term “effector cell ligand” as provided herein refers to a cell surface molecule expressed on an effector cell of the immune system (e.g., a cytotoxic T cell, a helper T cell, a B cell, a natural killer cell). Upon binding of the antibody to the effector cell ligand expressed on the effector cell, the effector cell is activated and able to exert its function (e.g., selective killing or eradication of malignant, infected or otherwise unhealthy cells). In embodiments, the effector cell ligand is a CD3 protein. In embodiments, the effector cell ligand is a CD16 protein. In embodiments, the effector cell ligand is a CD32 protein. In embodiments, the effector cell ligand is a NKp46 protein.

“Non-immunogenic” refers to a material that does not initiate, provoke or enhance an immune response where the immune response includes the adaptive and/or innate immune responses.

“Receptor” means a polypeptide that is capable of specific binding to a molecule. Whereas many receptors may typically operate on the surface of a cell, some receptors may bind ligands when located inside the cell (and prior to transport to the surface) or may reside predominantly intra-cellularly and bind ligand therein.

The term “truncated EGFR” or “tEGFR” as used herein refers to an EGFR protein that includes EGFR domain IV, and does not include the membrane distal EGF-binding domains I, II or III or the cytoplasmic signaling tail. In embodiments, the tEGFR does not include EGFR domain I, EGFR domain II, EGFR domain II and the cytoplasmic signaling tail.

The term “EGFR domain IV” or “domain IV EGFR” refers to the amino acid sequence typically found between domain III and the transmembrane domain of recombinant or naturally occurring forms of EGFR protein. In embodiments, EGFR domain IV, or variants or homologs thereof have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR domain IV.

In embodiments, the domain IV EGFR is substantially identical to the domain IV of the protein identified by the UniProt reference number P00533 or a variant or homolog having substantial identity thereto. In embodiments, the domain IV EGFR has at least 90%, 95%, 96%, 97%, 98%, 99% or 100% amino acid sequence identity across the whole sequence or a portion of the sequence (e.g. a 50, 100, 150 or 200 continuous amino acid portion) of the sequence of SEQ ID NO:276. In embodiments, the EGFR domain IV is substantially identical to the amino acid sequence identified by SEQ ID NO:276.

SEQ ID NO: 276:
VCHALCSPEGCWGPEPRDCVSCRNVSRGRECVDKCNLLEGEPREF
VENSECIQCHPECLPQAMNITCTGRGPDNCIQCAHYIDGPHCVKT
CPAGVMGENNTLVWKYADAGHVCHLCHPNCTYGCTGPGLEGCPTN
GPKIPS.

The term “EGFR domain III” or “domain III EGFR” refers to the amino acid sequence typically found between domain II EGFR and domain IV EGFR of recombinant or naturally occurring forms of EGFR protein. In embodiments, EGFR domain III, or variants or homologs thereof have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR domain III.

As used herein, “chimeric antigen receptor” is used according to its conventional meaning in the art refers to a recombinant protein including an antibody region and a transmembrane region.

An “antibody region” as provided herein refers to a monovalent or multivalent protein moiety that forms part of an antibody. A person of ordinary skill in the art would therefor immediately recognize that the antibody region is a protein moiety capable of binding an antigen (epitope). Thus, the antibody region provided herein may include a domain of an antibody or fragment (e.g., Fab) thereof. In embodiments, the antibody region includes a variable light chain domain and a variable heavy chain domain. A “variable light chain domain” as provided herein refers to a polypeptide including a light chain variable (VL) region. In embodiments, the variable light chain domain is a light chain variable (VL) region. A “variable heavy chain domain” as provided herein refers to a polypeptide including a heavy chain variable (VH) region. In embodiments, the variable heavy chain domain is a heavy chain variable (VH) region.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein (e.g., the tEGFR surface molecule, chimeric antigen receptor) including embodiments thereof are contemplated. Non-limiting examples of transmembrane domains include the transmembrane domains of CD28, CD8, CD4 or CD3-zeta.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain is a CD3 ζ intracellular T-cell signaling domain.

As used herein, “cancer antigen” refers to a molecule expressed on a cancer cell. In embodiments, the cancer antigen is expressed at a higher level relative to a standard control. IN embodiments, the cancer antigen is expressed on a healthy cell. A “standard control” can be the level of expression of the cancer antigen of a healthy cell. The standard control may be the expression level of the cancer antigen in a cell from a healthy subject (i.e. a subject that does not have cancer). The standard control may be the expression level of a non-cancerous cell derived from the same subject as the cancer antigen expressing cancer. In embodiments, the standard control is an expression level of a low cancer antigen or cancer antigen negative cancer cell. For example, the standard control can be the expression level of a biological sample comprising healthy cells (i.e. non-cancer cells). The standard control can be the expression level of cells from a subject that has already been treated for a cancer antigen expressing cancer. In instances, the control value can be obtained from the same subject (i.e. from a later-obtained sample, subsequent to treatment of the cancer antigen expressing cancer). The standard control can also represent an average expression level gathered from a population of similar subjects (i.e. healthy individuals with a similar medical background, same age, weight, etc.). In embodiments, the expression level of a cancer antigen is at least 2-fold higher than the expression level of a standard control. In embodiments, the expression level of a cancer antigen is at least 5, 10, 50, 100, 150, 200, 250, 300, 350, 400, 450, 500, or 1,000-fold higher than the expression level of a standard control. In embodiments, the expression level of a cancer antigen is 5, 10, 50, 100, 200, 300, 400, 500, 1,000, 10,000 or 100,000-fold higher than the expression level of a standard control.

The term “CD19 protein” or “CD19” as used herein includes any of the recombinant or naturally-occurring forms of B-lymphocyte antigen CD19, also known as CD19 molecule (Cluster of Differentiation 19), B-Lymphocyte Surface Antigen B4, T-Cell Surface Antigen Leu-12 and CVID3, or variants or homologs thereof that maintain CD19 activity (e.g. within at least 50%, 80%, 90%, 95%, 96%, 97%, 98%, 99% or 100% activity compared to CD19). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 protein. In embodiments, the CD19 protein is substantially identical to the protein identified by the UniProt reference number P15391 or a variant or homolog having substantial identity thereto.

The term “IL-15 protein” or “IL-15” as used herein includes any of the recombinant or naturally-occurring forms of Interleukin-15 (IL-15), or variants or homologs thereof that maintain IL-15 activity (e.g. within at least 50%, 80%, 90%, 95%, 96%, 97%, 98%, 99% or 100% activity compared to IL-15). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference number P40933 or a variant or homolog having substantial identity thereto.

As used herein, “self-cleaving peptidyl sequence” refers to a class of peptide sequences that can induce ribosomal skipping during translation, which results in the generation of multiple peptides originally encoded by a single mRNA. In embodiments, the self-cleaving peptidyl sequence is a T2A sequence. In embodiments, the self-cleaving peptidyl sequence is a T2A sequence or a 2A sequence. In embodiments, the self-cleaving peptidyl sequence is a foot-and-mouth disease virus sequence. In embodiments, the self-cleaving peptidyl sequence is PVKQLLNFDLLKLAGDVESNPGP. In embodiments, the self-cleaving peptidyl sequence is an equine rhinitis A virus sequence. In embodiments, the self-cleaving peptidyl sequence is QCTNYALLKLAGDVESNPGP. In embodiments, the self-cleaving peptidyl sequence is a porcine teschovirus 1 sequence. In embodiments, the self-cleaving peptidyl sequence is ATNFSLLKQAGDVEENPGP. In embodiments, the self-cleaving peptidyl sequence is Thosea asigna virus sequence. In embodiments, the self-cleaving peptidyl sequence is EGRGSLLTCGDVESNPGP.

### Modified Cell-Surface Molecules

In one embodiment, a gene encoding a modified endogenous cell-surface molecule (e.g., tEGFR) that may be used as a non-immunogenic selection epitope compatible with immunomagnetic selection is provided. Such a non-immunogenic selection epitope may facilitate immunotherapy in cancer patients without undesirable immunologic rejection of cell products. The endogenous cell surface molecule (e.g., tEGFR) may be modified or truncated to retain an extracellular epitope recognized by an anti-domain IV antibody or functional fragment thereof (e.g., the anti-domain IV antibody provided herein), and to remove any signaling or trafficking domains and/or any extracellular domains unrecognized by said anti-domain IV antibody. A modified endogenous cell surface molecule (e.g., tEGFR) which lacks a signaling or trafficking domain and/or any extracellular domains unrecognized by said anti-domain IV antibody is rendered inert.

The modified endogenous cell-surface molecule (e.g., tEGFR) may be, but is not limited to, any non-immunogenic cell-surface related receptor, glycoprotein, cell adhesion molecule, antigen, integrin or cluster of differentiation (CD) that is modified as described herein. Modification of such cell-surface molecules is accomplished by keeping an epitope that is recognized by an anti-domain IV antibody (e.g., the anti-domain IV antibody provided herein) or functional fragment thereof; and removing any signaling or trafficking domains and/or any extracellular domains unrecognized by an anti-domain IV antibody (e.g., the anti-domain IV antibody provided herein). Removal of the signaling or trafficking domains and/or any extracellular domains unrecognized by an anti-domain IV antibody (e.g., the anti-domain IV antibody provided herein) renders the endogenous cell-surface molecule non-immunogenic and/or inert.

Examples of endogenous cell-surface molecules that may be modified or truncated according to the embodiments described herein include, but are not limited to EpCAM, VEGFR, integrins (e.g., integrins .alpha . . . nu . . . beta.3, .alpha.4, .alpha . . . PI.b.beta.3, .alpha.4.beta. 7, .alpha.5.beta. 1, .alpha . . . nu . . . beta.3, .alpha . . . nu.), TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, or clusters of differentiation (e.g., CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7).

Corresponding antibodies that may be used to recognize a modified or truncated endogenous cell-surface molecule (e.g., tEGFR) include any of the antibodies provided herein including embodiments thereof.

In some embodiments, the modified endogenous cell-surface molecule (e.g., tEGFR) is encoded by a modified or truncated tyrosine kinase receptor gene. Examples of tyrosine kinase receptors that may be modified or truncated according to the embodiments described herein include, but are not limited to, members of the endothelial growth factor receptor family (EGRF/ErbB1/HER1; ErbB2/HER2/neu; ErbB3/HER3; ErbB4/HER4), hepatocyte growth factor receptor (HGFR/c-MET) and insulin-like growth factor receptor-1 (IGF-1R). According to some embodiments, modified tyrosine kinase receptors retain an extracellular epitope recognized by a known antibody or functional fragment thereof, and lack at least a tyrosine kinase domain. A modified tyrosine kinase receptor which lacks at least a tyrosine kinase domain renders the receptor inert.

In embodiments, the modified endogenous cell surface molecule is a truncated EGFR (tEGFR) that includes an EGFR domain IV and does not include an EGFR domain III. In embodiments, the tEGFR does not include Domain I, Domain II, Domain III, the Juxtamembrane Domain and the Tyrosine Kinase Domain as compared to an unmodified EGFR.

### Methods of Use

In another embodiment, a modified endogenous cell-surface molecule may be used as a marker for in vivo T cell engraftment. For example, when the modified endogenous cell-surface molecule is EGFRt, the EGFRt may be used to track the uptake of the T cells to which it is attached in vivo without affecting cellular function of the T cells or the cells to which the T cells are targeted, such as bone marrow cells in a transplant situation. The use of an anti-domain IV EGFR antibody as provided herein and conjugated to probes or reporter genes such as sr39TK may be used to improve the tracking potential of EGFRt-expressing cells to patients via PET imaging techniques.

In a separate embodiment, a modified endogenous cell-surface molecule may be used to induce cell suicide. For example, EGFRt may be used as a suicide gene via anti-domain IV EGFR antibody-mediated complement and/or antibody dependent cell mediated cytotoxicity (ADCC) pathways.

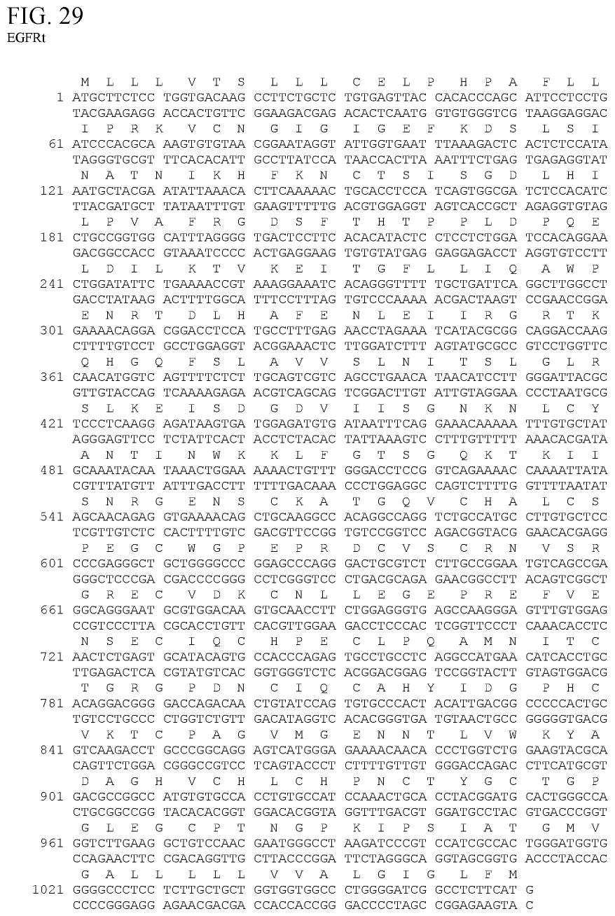

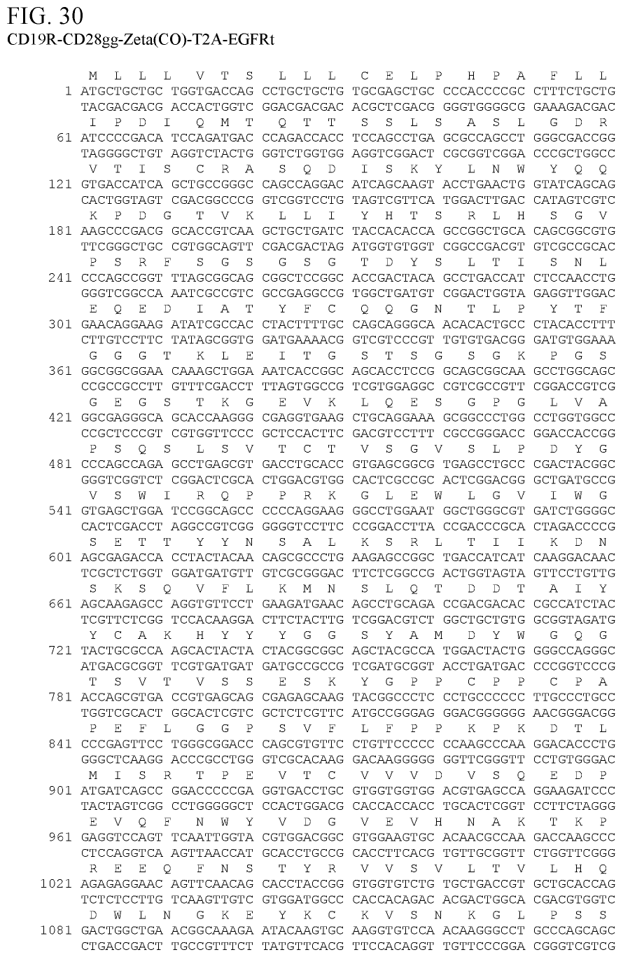

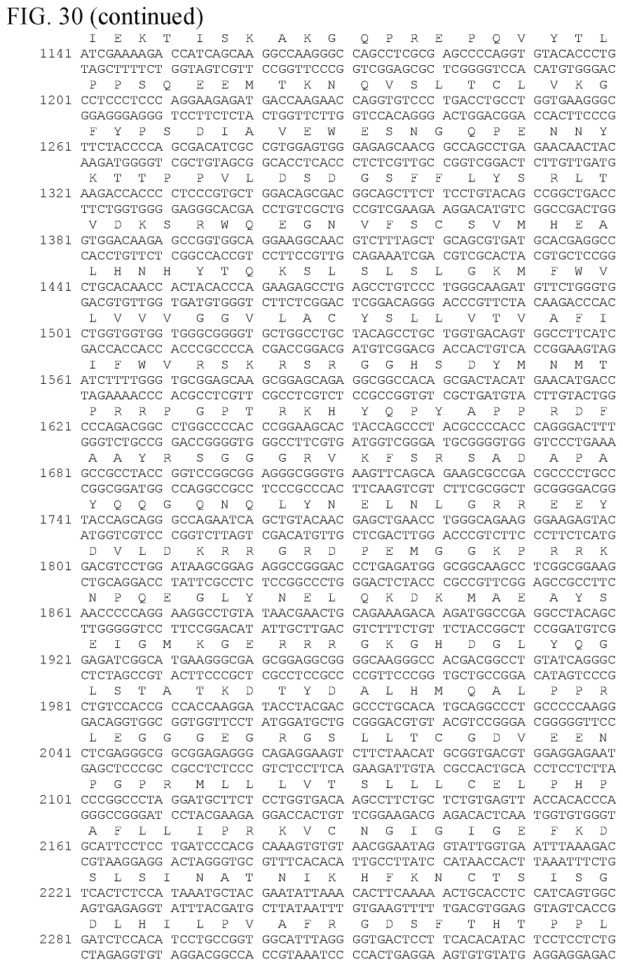

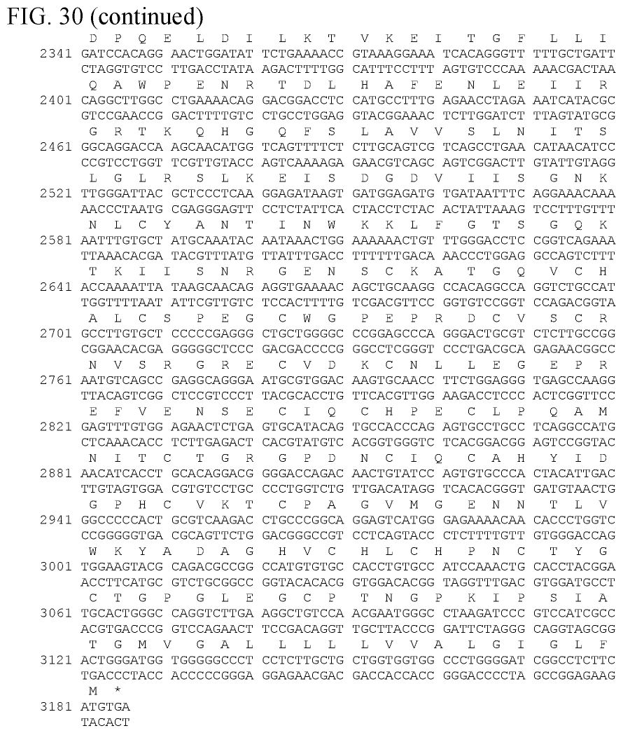

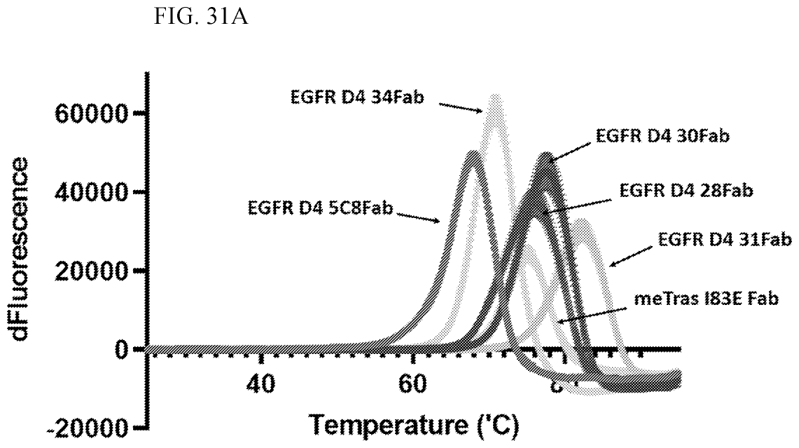

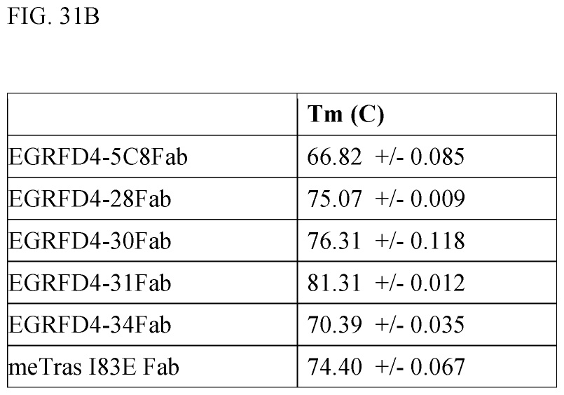

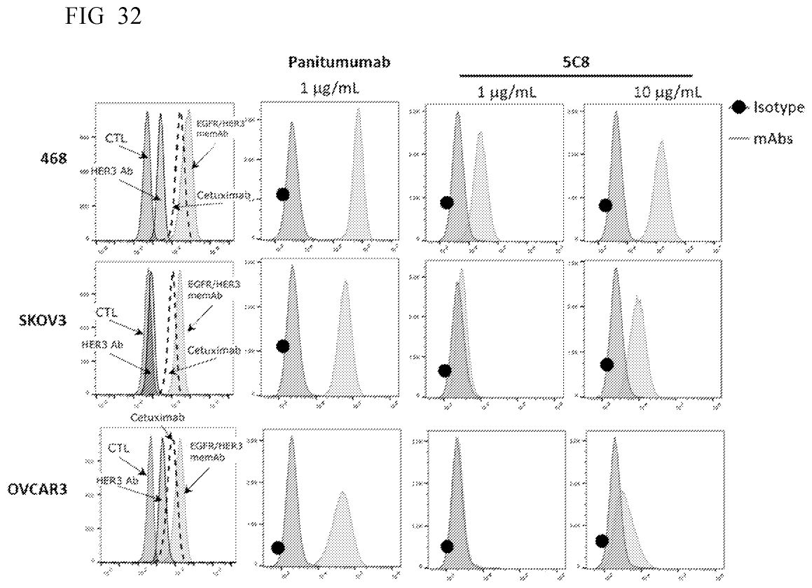

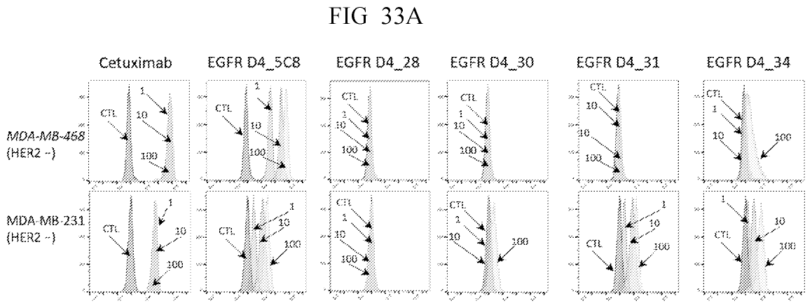

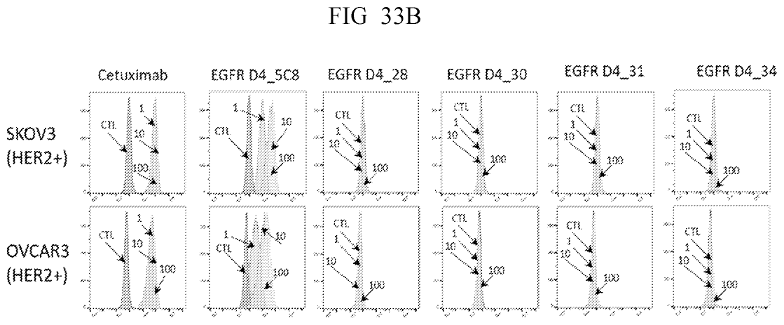

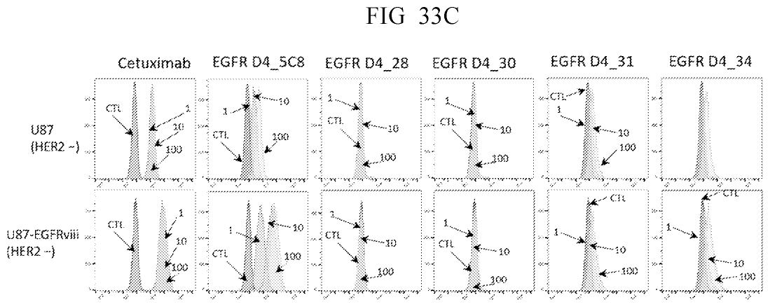

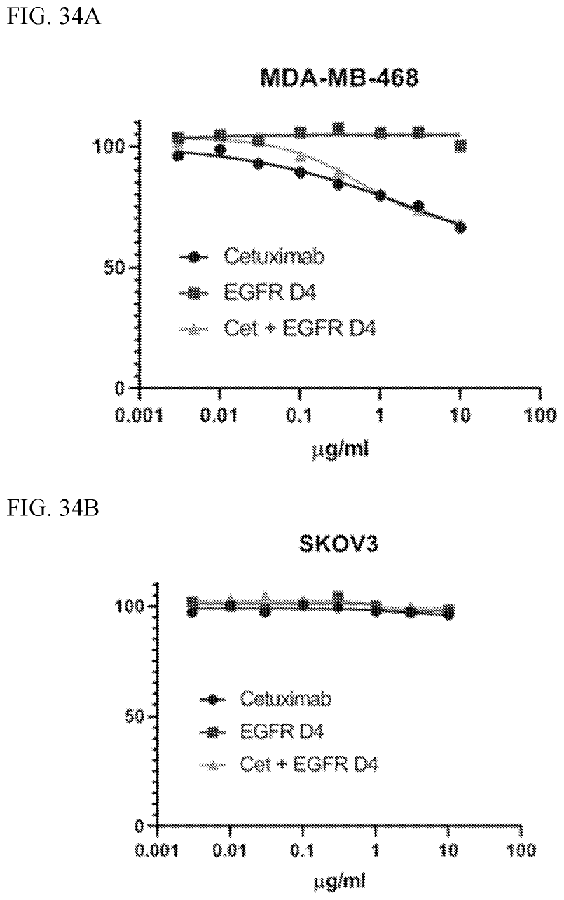

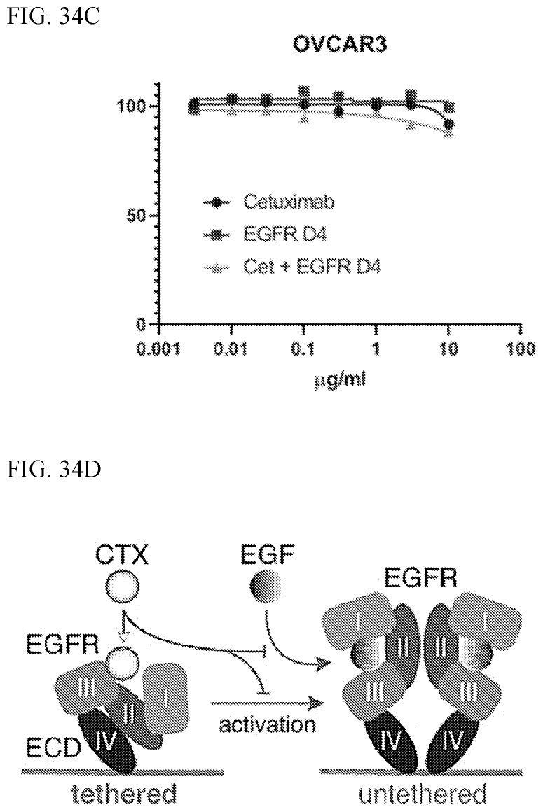

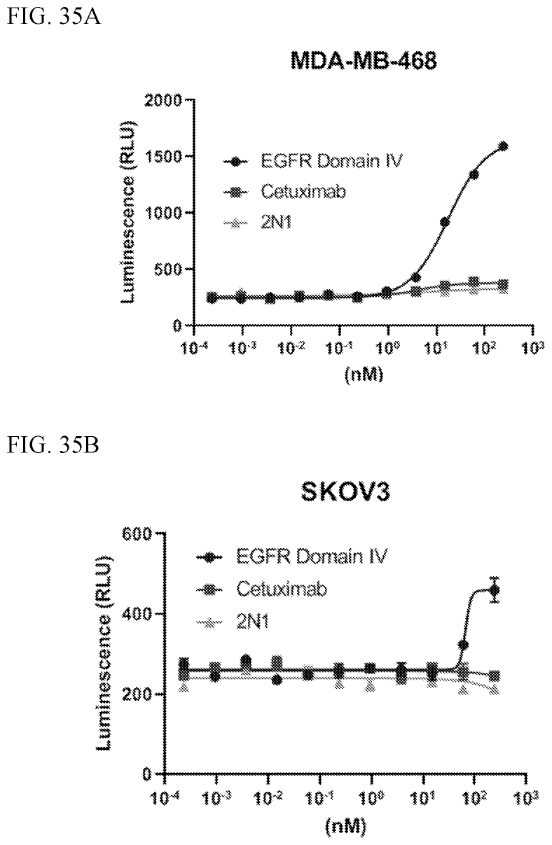

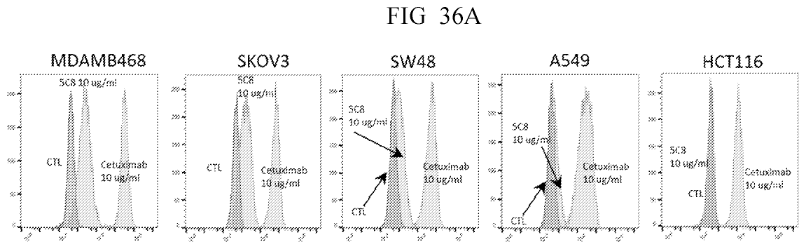

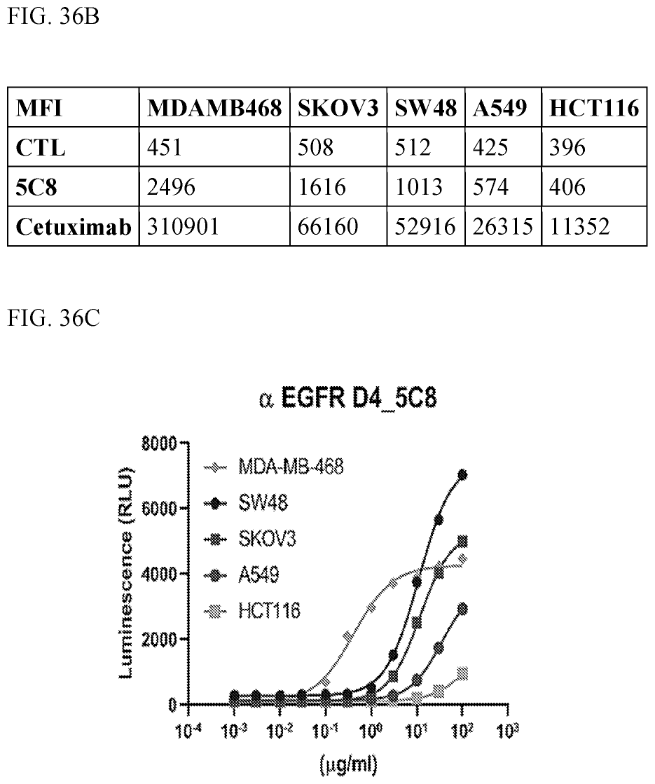

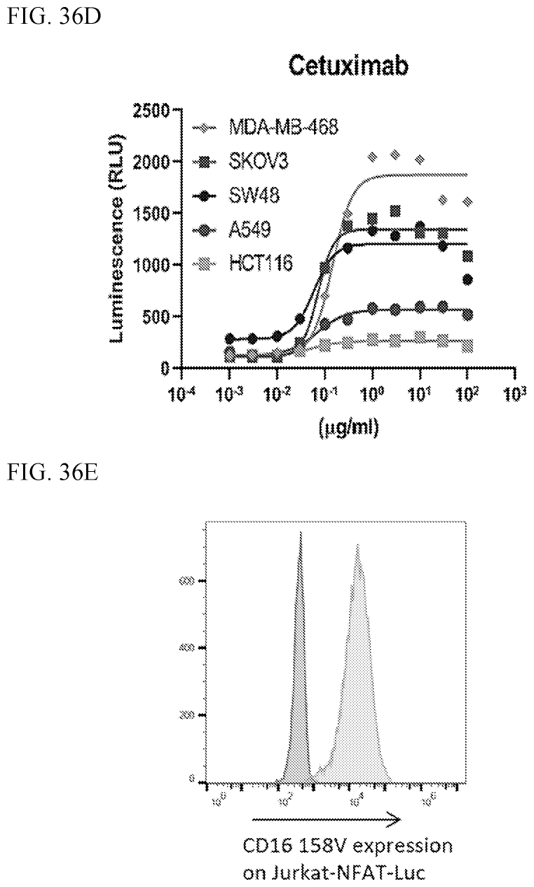

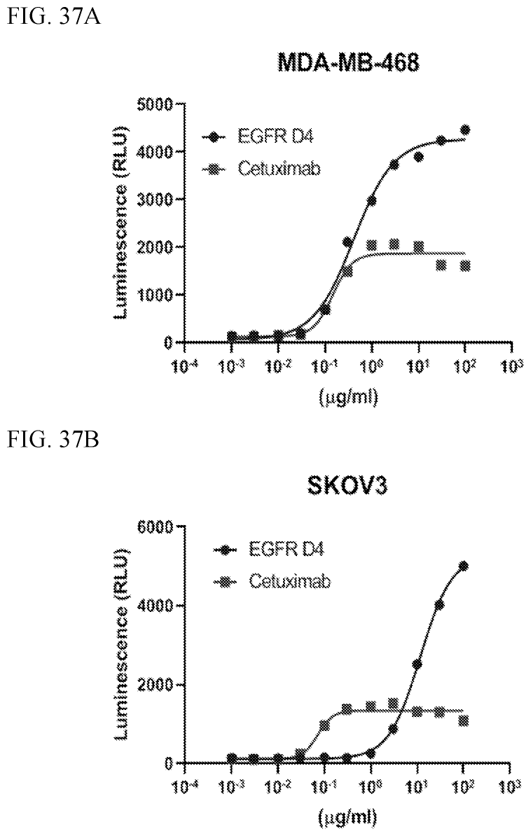

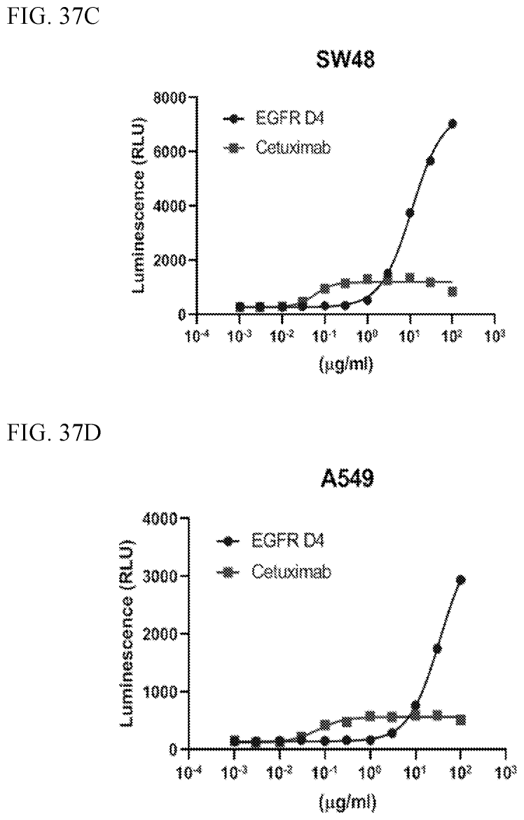

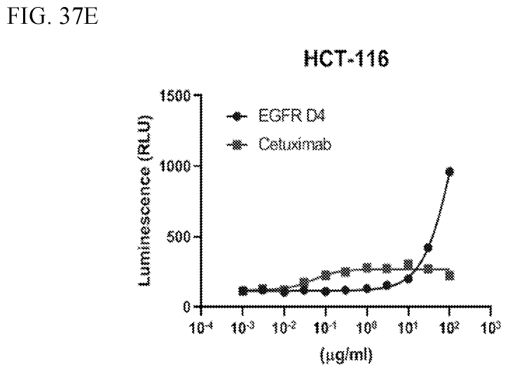

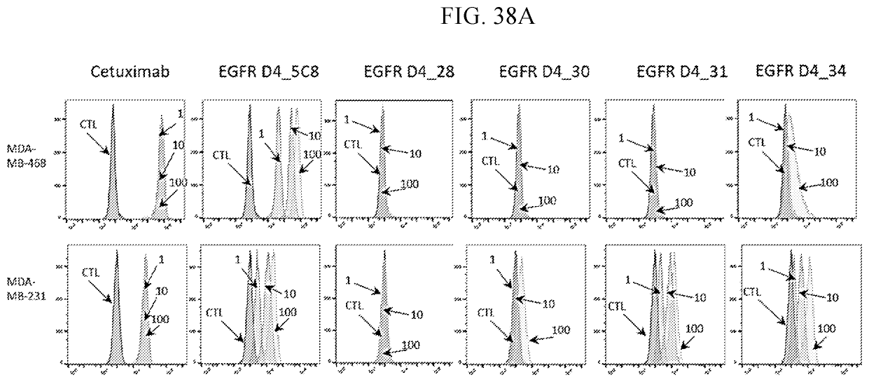

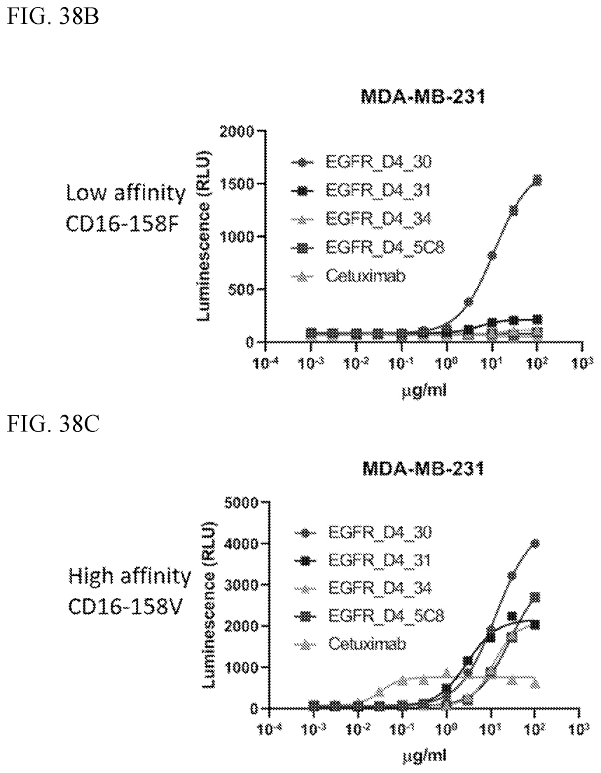

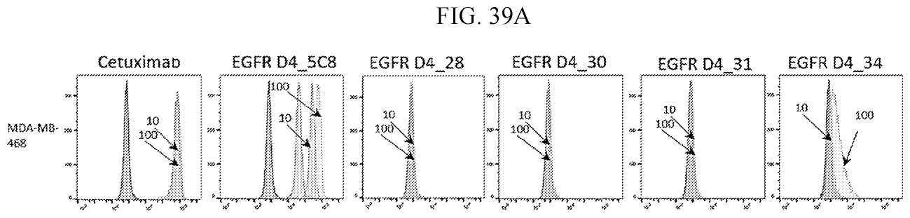

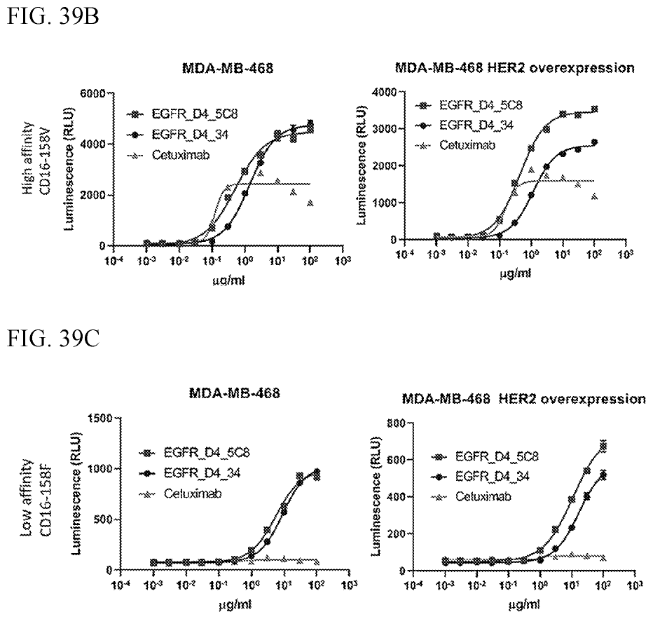

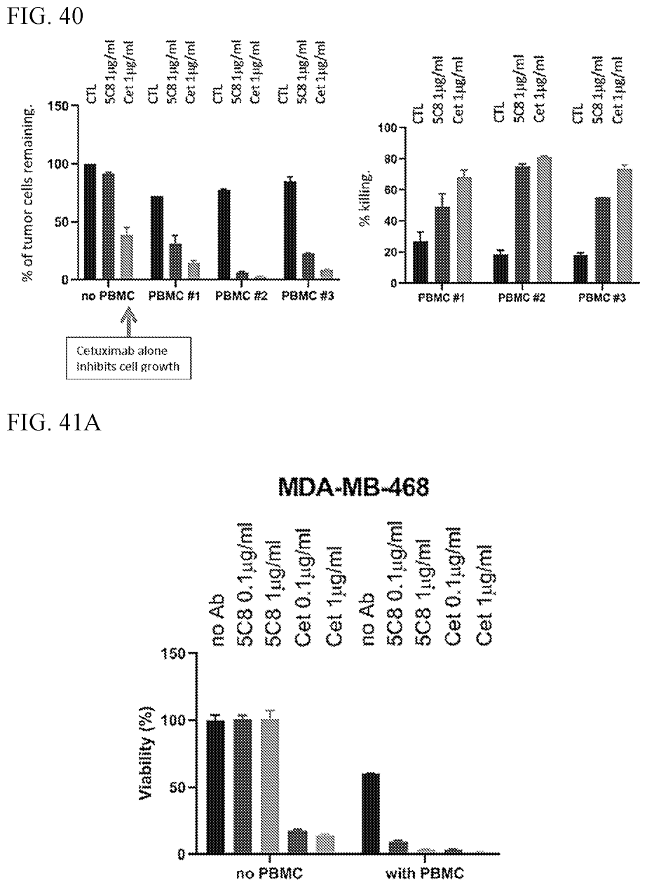

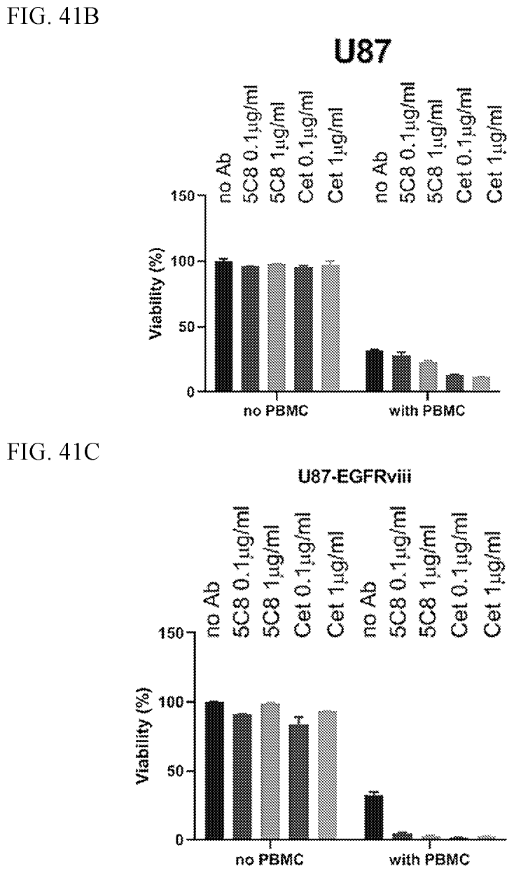

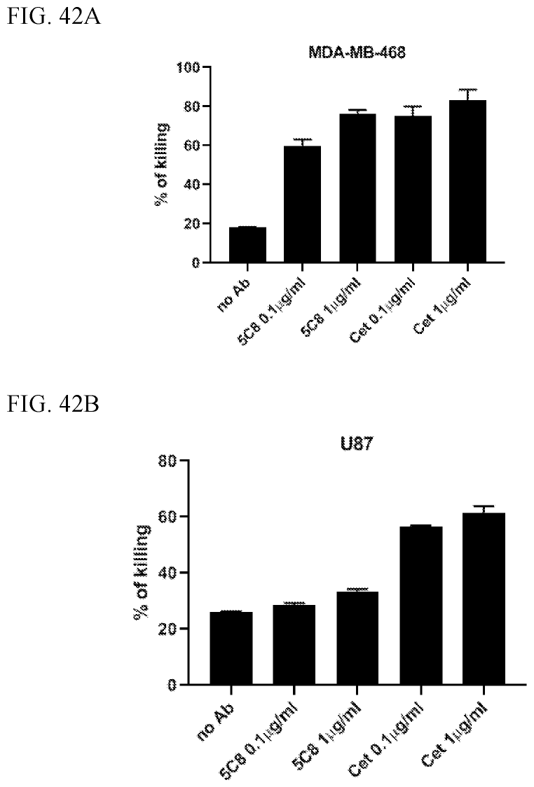

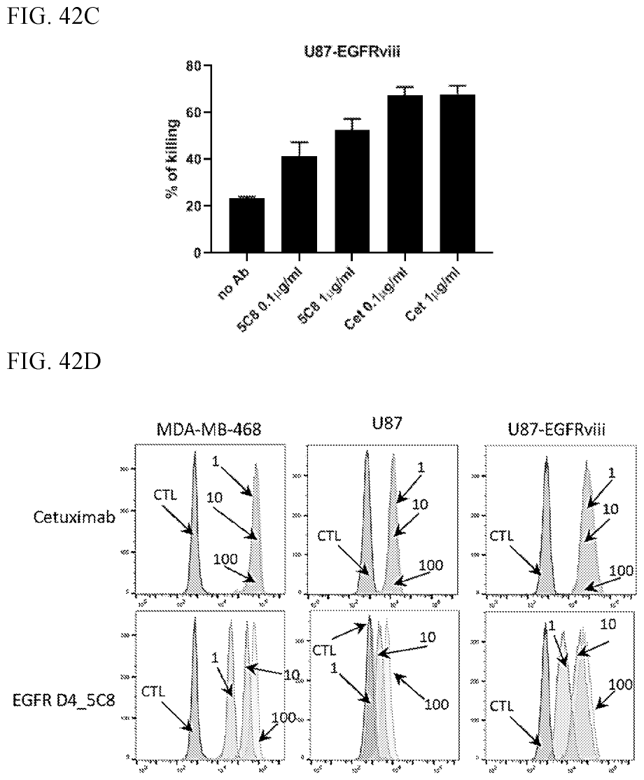

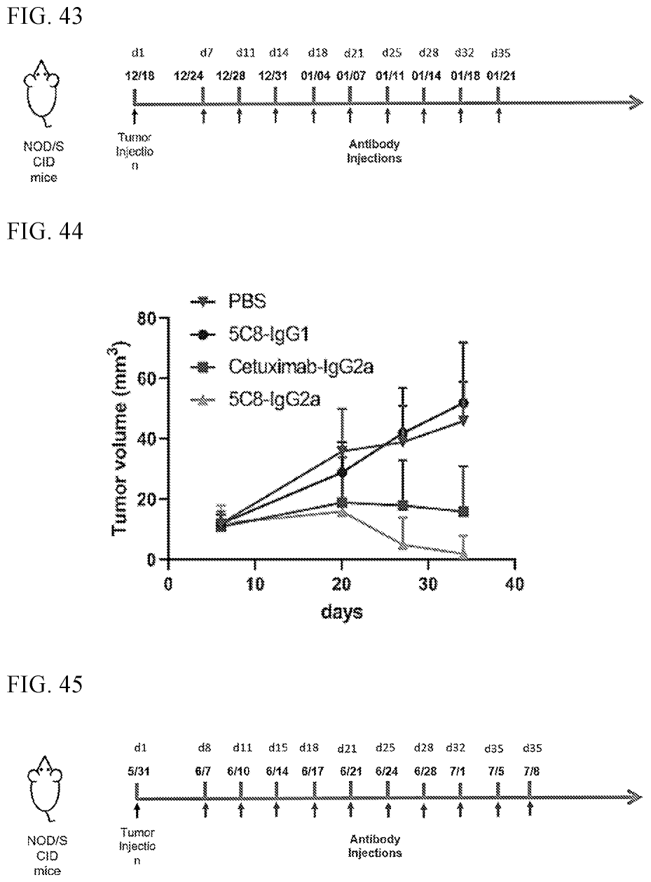

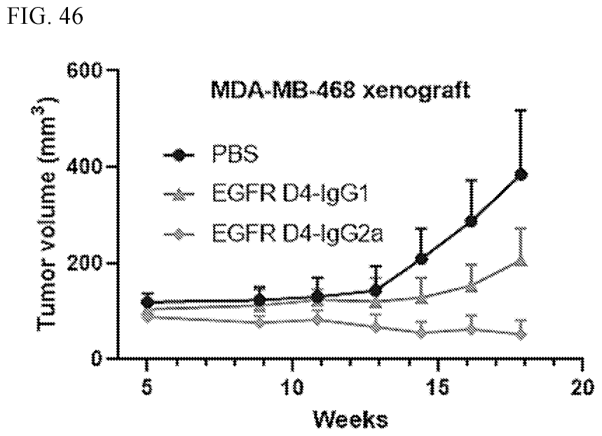

In other embodiments, the truncated epidermal growth factor receptor (EGFRt) selection epitope or other modified cell-surface molecule is attached to other sequences. One exemplary sequence is the GMCSFR alpha chain signal sequence, which directs surface expression, attached to EGFRt. GMCSFR is encoded by nucleotides 1-66 and EGFRt is encoded by nucleotides 67-1071. See FIG. 29. Also in FIG. 29 is the antisense strand and amino acid sequences of GMCSFR alpha chain signal sequence linked to EGFRt. Another such sequence is a codon-optimized cDNA sequence encoding an anti-CD19 costimulatory chimeric antigen receptor (CD19R-CD28gg-Zeta (CO)), and a cleavable T2A linker. Cytotoxic T lymphocytes (CTLs) modified to express a CD19-specific chimeric antigen receptor (CAR) that signals via a cytoplasmic costimulatory (CD28) domain fused to the cytoplasmic CD3-.zeta. domain exhibits superior anti-tumor potency that can be attributed to CD28-mediated survival and enhanced cytokine production. This construct may be further modified to incorporate a C-terminal 2A cleavable linker followed by the coding sequence for a truncated human EGFR (EGFRt) for the purpose of immunomagnetic purification of CAR-expressing transductants using anti-domain IV EGFR antibody-biotin/anti-biotin microbeads. See the CD19R-CD28gg-Zeta (CO)-T2A-EGFRt sequence attached as FIG. 30, (nucleotide sense strand), (nucleotide anti-sense strand), and (protein). Lentivector transduction of primary human T cells with this codon-optimized cDNA directs the coordinated expression of the CAR and EGFRt (FIG. 30).

To eliminate variability between transgene expression products otherwise intrinsic to transduction procedures without subsequent selection, a non-immunogenic selection epitope, EGFRt, compatible with immunomagnetic selection using the CliniMACS device (Miltenyi Biotec, Bergisch Gladbach, Germany) was developed. For example, EGFRt is a truncated human epidermal growth factor receptor that lacks the membrane distal EGF-binding domain and the ectoplasmic signaling tail, but retains the extracellular membrane proximal epitope recognized by the anti-domain IV EGFR antibody provided herein including embodiments thereof. Biotinylated-anti-domain IV EGFR antibody is applied to immunomagnetic selection in combination with anti-biotin microbeads (Miltenyi). Human OKT3 blasts that had been lentivirally transduced with CD19R-CD28gg-Zeta (CO)-T2A-EGFRt were subjected to immunomagnetic selection using the Miltenyi AutoMACS device, and the frequency of EGFRt+CAR+ T cells was enriched from 22% (pre-selection) to 99% (post-selection) without observable toxicity to the cell preparation. It is also possible that, instead of or in addition to immunomagnetic sorting, the EGFRt can be purified using fluorescence-based cell sorting techniques.

Due to the absence of the EGF-binding domains and intracellular signaling domains, EGFRt is inactive when expressed by T cells. Importantly, the EGFRt-selected T cells maintain their desired effector phenotype—including anti-tumor cyotoxic activity mediated by the chimeric antigen receptor that is coordinately expressed with the EGFRt—and remain amenable to established expansion protocols.

Overall, this EGFRt has various advantages for immunotherapeutic cell products compared to other selection markers that have been previously reported. Specifically, unlike truncated CD4 and CD19, it is not endogenously expressed by subpopulations of lymphocytes. Furthermore, in contrast to truncated CD34 and low affinity nerve growth factor receptor, it does not have any activity that might negatively affect the immune cell product (i.e., in terms of signaling or trafficking). Lastly, it alone can be bound/recognized by any of the anti-domain IV EGFR antibodies provided herein including embodiments thereof. Together, these attributes make the EGFRt provided herein a superior selection marker for any transfection/transduction system that can be applied to the generation of cell products for adoptive immunotherapy. Thus, EGFRt is well suited to be used as a selection marker for, e.g., lentivirally transduced T cells of immunotherapeutic relevance.

### Anti-Domain IV Egfr Antibodies

Provided herein are, inter alia, novel antibodies that specifically bind to domain IV of EGFR and are able to effectively induce antibody dependent cell mediated cytoxicity (ADCC) against EGFR-expressing cells. In embodiments, the immunoglobulin and the Fab of the antibodies provided herein bind domain IV of EGFR with differential affinity. In further embodiments, the Fab of an antibody provided herein binds domain IV EGFR with lower affinity than the IgG of the same antibody. Therefore, and without being bound to any specific theory, the antibodies provided herein may be capable of selectively binding EGFR high expressing cells (e.g., cancer cells), thereby providing for highly specific antibody therapeutics without adverse effects. Further provided herein are EGFR antibodies capable of binding truncated domain IV EGFR but substantially not binding to full-length EGFR. The term “full-length EGFR” as provided herein refers to endogenous EGFR, which includes domain I, domain II, domain III and domain IV. In contrast to full-length EGFR, a truncated domain IV EGFR as provided herein refers to a EGFR peptide, which includes domain IV, but does not include domain I, domain II or domain II EGFR.

In an aspect is provided an anti-epidermal growth factor receptor (EGFR) antibody capable of binding a truncated domain IV EGFR and not substantially binding to EGFR comprising domain I, domain II, domain II and domain IV.

In an aspect is provided an anti-epidermal growth factor receptor (EGFR) antibody capable of binding EGFR at a higher K_{D} relative to an antibody capable of binding domain I of EGFR, domain II of EGFR or domain III of EGFR.

In an aspect is provided an anti-epidermal growth factor receptor (EGFR) antibody including a heavy chain variable domain and a light chain variable domain, wherein the heavy chain variable domain includes any one of the combinations of a CDR1 sequence, a CDR2 sequence and a CDR3 sequence set forth by Table 1, 3, 5 or 7; and wherein the light chain variable domain includes any one of the combinations of a CDR1 sequence, a CDR2 sequence and a CDR3 sequence set forth by Table 2, 4, 6 or 8.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:16, a CDR H2 as set forth in SEQ ID NO:17 and a CDR H3 as set forth in SEQ ID NO:18; and wherein the light chain variable domain includes: a CDR L1 as set forth in SEQ ID NO:52, a CDR L2 as set forth in SEQ ID NO:53, and a CDR L3 as set forth in SEQ ID NO:54.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:19, a CDR H2 as set forth in SEQ ID NO:20 and a CDR H3 as set forth in SEQ ID NO:21; and wherein the light chain variable domain includes: a CDR L1 as set forth in SEQ ID NO:55, a CDR L2 as set forth in SEQ ID NO:56, and a CDR L3 as set forth in SEQ ID NO:57.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:22, a CDR H2 as set forth in SEQ ID NO:23 and a CDR H3 as set forth in SEQ ID NO:24; and wherein the light chain variable domain includes: a CDR L1 as set forth in SEQ ID NO:58, a CDR L2 as set forth in SEQ ID NO:59, and a CDR L3 as set forth in SEQ ID NO:60.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:25, a CDR H2 as set forth in SEQ ID NO:26 and a CDR H3 as set forth in SEQ ID NO:27; and wherein the light chain variable domain includes: a CDR L1 as set forth in SEQ ID NO:61, a CDR L2 as set forth in SEQ ID NO:62, and a CDR L3 as set forth in SEQ ID NO:63.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:34, a CDR H2 as set forth in SEQ ID NO:35 and a CDR H3 as set forth in SEQ ID NO:36; and wherein the light chain variable domain includes: a CDR L1 as set forth in SEQ ID NO:70, a CDR L2 as set forth in SEQ ID NO:71, and a CDR L3 as set forth in SEQ ID NO:72.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:85, a CDR H2 as set forth in SEQ ID NO:86 and a CDR H3 as set forth in SEQ ID NO:87; and wherein the light chain variable domain includes: a CDR L1 as set forth in SEQ ID NO:118, a CDR L2 as set forth in SEQ ID NO:119, and a CDR L3 as set forth in SEQ ID NO:120.

In an aspect is provided an anti-epidermal growth factor receptor (EGFR) antibody comprising a heavy chain variable domain and a light chain variable domain, wherein the heavy chain variable domain includes: a CDR H1 as set forth in SEQ ID NO:88, a CDR H2 as set forth in SEQ ID NO:89 and a CDR H3 as set forth in SEQ ID NO:90; and wherein the light chain variable domain includes: a CDR L1 as set forth in SEQ ID NO:121, a CDR L2 as set forth in SEQ ID NO:122, and a CDR L3 as set forth in SEQ ID NO:123.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:94, a CDR H2 as set forth in SEQ ID NO:95 and a CDR H3 as set forth in SEQ ID NO:96; and wherein the light chain variable domain includes: a CDR L1 as set forth in SEQ ID NO:127, a CDR L2 as set forth in SEQ ID NO:128, and a CDR L3 as set forth in SEQ ID NO:129.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:97, a CDR H2 as set forth in SEQ ID NO:98 and a CDR H3 as set forth in SEQ ID NO:99; and wherein the light chain variable domain includes: a CDR L1 as set forth in SEQ ID NO:130, a CDR L2 as set forth in SEQ ID NO:131, and a CDR L3 as set forth in SEQ ID NO:132.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:100, a CDR H2 as set forth in SEQ ID NO:101 and a CDR H3 as set forth in SEQ ID NO:102; and wherein the light chain variable domain includes: a CDR L1 as set forth in SEQ ID NO:133, a CDR L2 as set forth in SEQ ID NO:134, and a CDR L3 as set forth in SEQ ID NO:135. In embodiments, the antibody includes a heavy chain sequence of SEQ ID NO:247 and a light chain sequence of SEQ ID NO:248.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:103, a CDR H2 as set forth in SEQ ID NO:104 and a CDR H3 as set forth in SEQ ID NO:105; and wherein the light chain variable domain includes: a CDR L1 as set forth in SEQ ID NO:136, a CDR L2 as set forth in SEQ ID NO:137, and a CDR L3 as set forth in SEQ ID NO:138. In embodiments, the antibody includes a heavy chain sequence of SEQ ID NO:249 and a light chain sequence of SEQ ID NO:250.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:139, a CDR H2 as set forth in SEQ ID NO:140 and a CDR H3 as set forth in SEQ ID NO:141; and wherein the light chain variable domain includes: a CDR L1 as set forth in SEQ ID NO:169, a CDR L2 as set forth in SEQ ID NO:170, and a CDR L3 as set forth in SEQ ID NO:171.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:145, a CDR H2 as set forth in SEQ ID NO:146 and a CDR H3 as set forth in SEQ ID NO:147; and wherein the light chain variable domain includes: a CDR L1 as set forth in SEQ ID NO:175, a CDR L2 as set forth in SEQ ID NO:176, and a CDR L3 as set forth in SEQ ID NO:177.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:148, a CDR H2 as set forth in SEQ ID NO:149 and a CDR H3 as set forth in SEQ ID NO:150; and wherein the light chain variable domain includes: a CDR L1 as set forth in SEQ ID NO:178, a CDR L2 as set forth in SEQ ID NO:179, and a CDR L3 as set forth in SEQ ID NO:180. In embodiments, the antibody includes a heavy chain sequence of SEQ ID NO:257 and a light chain sequence of SEQ ID NO:258.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:154, a CDR H2 as set forth in SEQ ID NO:155 and a CDR H3 as set forth in SEQ ID NO:156; and wherein the light chain variable domain includes: a CDR L1 as set forth in SEQ ID NO:184, a CDR L2 as set forth in SEQ ID NO:185, and a CDR L3 as set forth in SEQ ID NO:186.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:157, a CDR H2 as set forth in SEQ ID NO:158 and a CDR H3 as set forth in SEQ ID NO:159; and wherein the light chain variable domain includes: a CDR L1 as set forth in SEQ ID NO:187, a CDR L2 as set forth in SEQ ID NO:188, and a CDR L3 as set forth in SEQ ID NO:189.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:163, a CDR H2 as set forth in SEQ ID NO:164 and a CDR H3 as set forth in SEQ ID NO:165; and wherein the light chain variable domain includes: a CDR L1 as set forth in SEQ ID NO:193, a CDR L2 as set forth in SEQ ID NO:194, and a CDR L3 as set forth in SEQ ID NO:195.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:166, a CDR H2 as set forth in SEQ ID NO:167 and a CDR H3 as set forth in SEQ ID NO:168; and wherein the light chain variable domain includes: a CDR L1 as set forth in SEQ ID NO:196, a CDR L2 as set forth in SEQ ID NO:197, and a CDR L3 as set forth in SEQ ID NO:198.

In an aspect is provided an anti-epidermal growth factor receptor (EGFR) antibody including a heavy chain variable domain and a light chain variable domain, wherein the heavy chain variable domain includes: a CDR H1 as set forth in SEQ ID NO:199, a CDR H2 as set forth in SEQ ID NO:200 and a CDR H3 as set forth in SEQ ID NO:201; and wherein the light chain variable domain includes: a CDR L1 as set forth in SEQ ID NO:202, a CDR L2 as set forth in SEQ ID NO:203, and a CDR L3 as set forth in SEQ ID NO:204. In embodiments, the antibody includes a heavy chain sequence of SEQ ID NO:271 and a light chain sequence of SEQ ID NO:272.

In embodiments, the antibody includes a heavy chain variable domain including any one of the combinations of a CDR1 sequence, a CDR2 sequence and a CDR3 sequence set forth by Table 1 and a light chain variable domain including any one of the combinations of a CDR1 sequence, a CDR2 sequence and a CDR3 sequence set forth by Table 2. In embodiments, the antibody includes a heavy chain variable domain including any one of the combinations of a CDR1 sequence, a CDR2 sequence and a CDR3 sequence set forth by Table 1 and a light chain variable domain including any one of the combinations of a CDR1 sequence, a CDR2 sequence and a CDR3 sequence set forth by Table 4. In embodiments, the antibody includes a heavy chain variable domain including any one of the combinations of a CDR1 sequence, a CDR2 sequence and a CDR3 sequence set forth by Table 1 and a light chain variable domain including any one of the combinations of a CDR1 sequence, a CDR2 sequence and a CDR3 sequence set forth by Table 6. In embodiments, the antibody includes a heavy chain variable domain including any one of the combinations of a CDR1 sequence, a CDR2 sequence and a CDR3 sequence set forth by Table 1 and a light chain variable domain including any one of the combinations of a CDR1 sequence, a CDR2 sequence and a CDR3 sequence set forth by Table 8.

In embodiments, the antibody includes a heavy chain variable domain including any one of the combinations of a CDR1 sequence, a CDR2 sequence and a CDR3 sequence set forth by Table 3 and a light chain variable domain including any one of the combinations of a CDR1 sequence, a CDR2 sequence and a CDR3 sequence set forth by Table 2. In embodiments, the antibody includes a heavy chain variable domain including any one of the combinations of a CDR1 sequence, a CDR2 sequence and a CDR3 sequence set forth by Table 3 and a light chain variable domain including any one of the combinations of a CDR1 sequence, a CDR2 sequence and a CDR3 sequence set forth by Table 4. In embodiments, the antibody includes a heavy chain variable domain including any one of the combinations of a CDR1 sequence, a CDR2 sequence and a CDR3 sequence set forth by Table 3 and a light chain variable domain including any one of the combinations of a CDR1 sequence, a CDR2 sequence and a CDR3 sequence set forth by Table 6. In embodiments, the antibody includes a heavy chain variable domain including any one of the combinations of a CDR1 sequence, a CDR2 sequence and a CDR3 sequence set forth by Table 3 and a light chain variable domain including any one of the combinations of a CDR1 sequence, a CDR2 sequence and a CDR3 sequence set forth by Table 8.

In embodiments, the antibody includes a heavy chain variable domain including any one of the combinations of a CDR1 sequence, a CDR2 sequence and a CDR3 sequence set forth by Table 5 and a light chain variable domain including any one of the combinations of a CDR1 sequence, a CDR2 sequence and a CDR3 sequence set forth by Table 2. In embodiments, the antibody includes a heavy chain variable domain including any one of the combinations of a CDR1 sequence, a CDR2 sequence and a CDR3 sequence set forth by Table 5 and a light chain variable domain including any one of the combinations of a CDR1 sequence, a CDR2 sequence and a CDR3 sequence set forth by Table 4. In embodiments, the antibody includes a heavy chain variable domain including any one of the combinations of a CDR1 sequence, a CDR2 sequence and a CDR3 sequence set forth by Table 5 and a light chain variable domain including any one of the combinations of a CDR1 sequence, a CDR2 sequence and a CDR3 sequence set forth by Table 6. In embodiments, the antibody includes a heavy chain variable domain including any one of the combinations of a CDR1 sequence, a CDR2 sequence and a CDR3 sequence set forth by Table 5 and a light chain variable domain including any one of the combinations of a CDR1 sequence, a CDR2 sequence and a CDR3 sequence set forth by Table 8.

In embodiments, the antibody includes a heavy chain variable domain including any one of the combinations of a CDR1 sequence, a CDR2 sequence and a CDR3 sequence set forth by Table 7 and a light chain variable domain including any one of the combinations of a CDR1 sequence, a CDR2 sequence and a CDR3 sequence set forth by Table 2. In embodiments, the antibody includes a heavy chain variable domain including any one of the combinations of a CDR1 sequence, a CDR2 sequence and a CDR3 sequence set forth by Table 7 and a light chain variable domain including any one of the combinations of a CDR1 sequence, a CDR2 sequence and a CDR3 sequence set forth by Table 4. In embodiments, the antibody includes a heavy chain variable domain including any one of the combinations of a CDR1 sequence, a CDR2 sequence and a CDR3 sequence set forth by Table 7 and a light chain variable domain including any one of the combinations of a CDR1 sequence, a CDR2 sequence and a CDR3 sequence set forth by Table 6. In embodiments, the antibody includes a heavy chain variable domain including any one of the combinations of a CDR1 sequence, a CDR2 sequence and a CDR3 sequence set forth by Table 7 and a light chain variable domain including any one of the combinations of a CDR1 sequence, a CDR2 sequence and a CDR3 sequence set forth by Table 8.

In embodiments, the antibody has a heavy chain variable domain and a light chain variable domain, wherein the heavy chain variable domain includes: a CDR H1 as set forth in SEQ ID NO:1, a CDR H2 as set forth in SEQ ID NO:2 and a CDR H3 as set forth in SEQ ID NO:3; and wherein the light chain variable domain includes: a CDR L1 as set forth in SEQ ID NO:37, a CDR L2 as set forth in SEQ ID NO:38, and a CDR L3 as set forth in SEQ ID NO:39.

In embodiments, the antibody has a heavy chain variable domain and a light chain variable domain, wherein the heavy chain variable domain includes: a CDR H1 as set forth in SEQ ID NO:4, a CDR H2 as set forth in SEQ ID NO:5 and a CDR H3 as set forth in SEQ ID NO:6; and wherein the light chain variable domain includes: a CDR L1 as set forth in SEQ ID NO:40, a CDR L2 as set forth in SEQ ID NO:41, and a CDR L3 as set forth in SEQ ID NO:42.

In embodiments, the antibody has a heavy chain variable domain and a light chain variable domain, wherein the heavy chain variable domain includes: a CDR H1 as set forth in SEQ ID NO:7, a CDR H2 as set forth in SEQ ID NO:8 and a CDR H3 as set forth in SEQ ID NO:9; and wherein the light chain variable domain includes: a CDR L1 as set forth in SEQ ID NO:43, a CDR L2 as set forth in SEQ ID NO:44, and a CDR L3 as set forth in SEQ ID NO:45.

In embodiments, the antibody has a heavy chain variable domain and a light chain variable domain, wherein the heavy chain variable domain includes: a CDR H1 as set forth in SEQ ID NO:10, a CDR H2 as set forth in SEQ ID NO:11 and a CDR H3 as set forth in SEQ ID NO:12; and wherein the light chain variable domain includes: a CDR L1 as set forth in SEQ ID NO:46, a CDR L2 as set forth in SEQ ID NO:47, and a CDR L3 as set forth in SEQ ID NO:48.

In embodiments, the antibody has a heavy chain variable domain and a light chain variable domain, wherein the heavy chain variable domain includes: a CDR H1 as set forth in SEQ ID NO:13, a CDR H2 as set forth in SEQ ID NO:14 and a CDR H3 as set forth in SEQ ID NO:15; and wherein the light chain variable domain includes: a CDR L1 as set forth in SEQ ID NO:49, a CDR L2 as set forth in SEQ ID NO:50, and a CDR L3 as set forth in SEQ ID NO:51.

In embodiments, the antibody has a heavy chain variable domain and a light chain variable domain, wherein the heavy chain variable domain includes: a CDR H1 as set forth in SEQ ID NO:16, a CDR H2 as set forth in SEQ ID NO:17 and a CDR H3 as set forth in SEQ ID NO:18; and wherein the light chain variable domain includes: a CDR L1 as set forth in SEQ ID NO:52, a CDR L2 as set forth in SEQ ID NO:53, and a CDR L3 as set forth in SEQ ID NO:54.

In embodiments, the antibody has a heavy chain variable domain and a light chain variable domain, wherein the heavy chain variable domain includes: a CDR H1 as set forth in SEQ ID NO:19, a CDR H2 as set forth in SEQ ID NO:20 and a CDR H3 as set forth in SEQ ID NO:21; and wherein the light chain variable domain includes: a CDR L1 as set forth in SEQ ID NO:55, a CDR L2 as set forth in SEQ ID NO:56, and a CDR L3 as set forth in SEQ ID NO:57.

In embodiments, the antibody has a heavy chain variable domain and a light chain variable domain, wherein the heavy chain variable domain includes: a CDR H1 as set forth in SEQ ID NO:22, a CDR H2 as set forth in SEQ ID NO:23 and a CDR H3 as set forth in SEQ ID NO:24; and wherein the light chain variable domain includes: a CDR L1 as set forth in SEQ ID NO:58, a CDR L2 as set forth in SEQ ID NO:59, and a CDR L3 as set forth in SEQ ID NO:60.

In embodiments, the antibody has a heavy chain variable domain and a light chain variable domain, wherein the heavy chain variable domain includes: a CDR H1 as set forth in SEQ ID NO:25, a CDR H2 as set forth in SEQ ID NO:26 and a CDR H3 as set forth in SEQ ID NO:27; and wherein the light chain variable domain includes: a CDR L1 as set forth in SEQ ID NO:61, a CDR L2 as set forth in SEQ ID NO:62, and a CDR L3 as set forth in SEQ ID NO:63.

In embodiments, the antibody has a heavy chain variable domain and a light chain variable domain, wherein the heavy chain variable domain includes: a CDR H1 as set forth in SEQ ID NO:28, a CDR H2 as set forth in SEQ ID NO:29 and a CDR H3 as set forth in SEQ ID NO:30; and wherein the light chain variable domain includes: a CDR L1 as set forth in SEQ ID NO:64, a CDR L2 as set forth in SEQ ID NO:65, and a CDR L3 as set forth in SEQ ID NO:66.

In embodiments, the antibody has a heavy chain variable domain and a light chain variable domain, wherein the heavy chain variable domain includes: a CDR H1 as set forth in SEQ ID NO:31, a CDR H2 as set forth in SEQ ID NO:32 and a CDR H3 as set forth in SEQ ID NO:33; and wherein the light chain variable domain includes: a CDR L1 as set forth in SEQ ID NO:67, a CDR L2 as set forth in SEQ ID NO:68, and a CDR L3 as set forth in SEQ ID NO:69.

In embodiments, the antibody has a heavy chain variable domain and a light chain variable domain, wherein the heavy chain variable domain includes: a CDR H1 as set forth in SEQ ID NO:34, a CDR H2 as set forth in SEQ ID NO:35 and a CDR H3 as set forth in SEQ ID NO:36; and wherein the light chain variable domain includes: a CDR L1 as set forth in SEQ ID NO:70, a CDR L2 as set forth in SEQ ID NO:71, and a CDR L3 as set forth in SEQ ID NO:72.

In embodiments, the antibody has a heavy chain variable domain and a light chain variable domain, wherein the heavy chain variable domain includes: a CDR H1 as set forth in SEQ ID NO:73, a CDR H2 as set forth in SEQ ID NO:74 and a CDR H3 as set forth in SEQ ID NO:75; and wherein the light chain variable domain includes: a CDR L1 as set forth in SEQ ID NO:106, a CDR L2 as set forth in SEQ ID NO:107, and a CDR L3 as set forth in SEQ ID NO:108.

In embodiments, the antibody has a heavy chain variable domain and a light chain variable domain, wherein the heavy chain variable domain includes: a CDR H1 as set forth in SEQ ID NO:76, a CDR H2 as set forth in SEQ ID NO:77 and a CDR H3 as set forth in SEQ ID NO:78; and wherein the light chain variable domain includes: a CDR L1 as set forth in SEQ ID NO:109, a CDR L2 as set forth in SEQ ID NO:110, and a CDR L3 as set forth in SEQ ID NO:111.

In embodiments, the antibody has a heavy chain variable domain and a light chain variable domain, wherein the heavy chain variable domain includes: a CDR H1 as set forth in SEQ ID NO:79, a CDR H2 as set forth in SEQ ID NO:80 and a CDR H3 as set forth in SEQ ID NO:81; and wherein the light chain variable domain includes: a CDR L1 as set forth in SEQ ID NO:112, a CDR L2 as set forth in SEQ ID NO:113, and a CDR L3 as set forth in SEQ ID NO:114.

In embodiments, the antibody has a heavy chain variable domain and a light chain variable domain, wherein the heavy chain variable domain includes: a CDR H1 as set forth in SEQ ID NO:82, a CDR H2 as set forth in SEQ ID NO:83 and a CDR H3 as set forth in SEQ ID NO:84; and wherein the light chain variable domain includes: a CDR L1 as set forth in SEQ ID NO:115, a CDR L2 as set forth in SEQ ID NO:116, and a CDR L3 as set forth in SEQ ID NO:117.

In embodiments, the antibody has a heavy chain variable domain and a light chain variable domain, wherein the heavy chain variable domain includes: a CDR H1 as set forth in SEQ ID NO:85, a CDR H2 as set forth in SEQ ID NO:86 and a CDR H3 as set forth in SEQ ID NO:87; and wherein the light chain variable domain includes: a CDR L1 as set forth in SEQ ID NO:118, a CDR L2 as set forth in SEQ ID NO:119, and a CDR L3 as set forth in SEQ ID NO:120.

In embodiments, the antibody has a heavy chain variable domain and a light chain variable domain, wherein the heavy chain variable domain includes: a CDR H1 as set forth in SEQ ID NO:88, a CDR H2 as set forth in SEQ ID NO:89 and a CDR H3 as set forth in SEQ ID NO:90; and wherein the light chain variable domain includes: a CDR L1 as set forth in SEQ ID NO:121, a CDR L2 as set forth in SEQ ID NO:122, and a CDR L3 as set forth in SEQ ID NO:123.

In embodiments, the antibody has a heavy chain variable domain and a light chain variable domain, wherein the heavy chain variable domain includes: a CDR H1 as set forth in SEQ ID NO:91, a CDR H2 as set forth in SEQ ID NO:92 and a CDR H3 as set forth in SEQ ID NO:93; and wherein the light chain variable domain includes: a CDR L1 as set forth in SEQ ID NO:124, a CDR L2 as set forth in SEQ ID NO:125, and a CDR L3 as set forth in SEQ ID NO:126.

In embodiments, the antibody has a heavy chain variable domain and a light chain variable domain, wherein the heavy chain variable domain includes: a CDR H1 as set forth in SEQ ID NO:94, a CDR H2 as set forth in SEQ ID NO:95 and a CDR H3 as set forth in SEQ ID NO:96; and wherein the light chain variable domain includes: a CDR L1 as set forth in SEQ ID NO:127, a CDR L2 as set forth in SEQ ID NO:128, and a CDR L3 as set forth in SEQ ID NO:129.

In embodiments, the antibody has a heavy chain variable domain and a light chain variable domain, wherein the heavy chain variable domain includes: a CDR H1 as set forth in SEQ ID NO:97, a CDR H2 as set forth in SEQ ID NO:98 and a CDR H3 as set forth in SEQ ID NO:99; and wherein the light chain variable domain includes: a CDR L1 as set forth in SEQ ID NO:130, a CDR L2 as set forth in SEQ ID NO:131, and a CDR L3 as set forth in SEQ ID NO:132.

In embodiments, the antibody has a heavy chain variable domain and a light chain variable domain, wherein the heavy chain variable domain includes: a CDR H1 as set forth in SEQ ID NO:100, a CDR H2 as set forth in SEQ ID NO:101 and a CDR H3 as set forth in SEQ ID NO:102; and wherein the light chain variable domain includes: a CDR L1 as set forth in SEQ ID NO:133, a CDR L2 as set forth in SEQ ID NO:134, and a CDR L3 as set forth in SEQ ID NO:135.

In embodiments, the antibody has a heavy chain variable domain and a light chain variable domain, wherein the heavy chain variable domain includes: a CDR H1 as set forth in SEQ ID NO:103, a CDR H2 as set forth in SEQ ID NO:104 and a CDR H3 as set forth in SEQ ID NO:105; and wherein the light chain variable domain includes: a CDR L1 as set forth in SEQ ID NO:136, a CDR L2 as set forth in SEQ ID NO:137, and a CDR L3 as set forth in SEQ ID NO:138.

In embodiments, the antibody has a heavy chain variable domain and a light chain variable domain, wherein the heavy chain variable domain includes: a CDR H1 as set forth in SEQ ID NO:139, a CDR H2 as set forth in SEQ ID NO:140 and a CDR H3 as set forth in SEQ ID NO:141; and wherein the light chain variable domain includes: a CDR L1 as set forth in SEQ ID NO:169, a CDR L2 as set forth in SEQ ID NO:170, and a CDR L3 as set forth in SEQ ID NO:171.

In embodiments, the antibody has a heavy chain variable domain and a light chain variable domain, wherein the heavy chain variable domain includes: a CDR H1 as set forth in SEQ ID NO:142, a CDR H2 as set forth in SEQ ID NO:143 and a CDR H3 as set forth in SEQ ID NO:144; and wherein the light chain variable domain includes: a CDR L1 as set forth in SEQ ID NO:172, a CDR L2 as set forth in SEQ ID NO:173, and a CDR L3 as set forth in SEQ ID NO:174.

In embodiments, the antibody has a heavy chain variable domain and a light chain variable domain, wherein the heavy chain variable domain includes: a CDR H1 as set forth in SEQ ID NO:145, a CDR H2 as set forth in SEQ ID NO:146 and a CDR H3 as set forth in SEQ ID NO:147; and wherein the light chain variable domain includes: a CDR L1 as set forth in SEQ ID NO:175, a CDR L2 as set forth in SEQ ID NO:176, and a CDR L3 as set forth in SEQ ID NO:177.

In embodiments, the antibody has a heavy chain variable domain and a light chain variable domain, wherein the heavy chain variable domain includes: a CDR H1 as set forth in SEQ ID NO:148, a CDR H2 as set forth in SEQ ID NO:149 and a CDR H3 as set forth in SEQ ID NO:150; and wherein the light chain variable domain includes: a CDR L1 as set forth in SEQ ID NO:178, a CDR L2 as set forth in SEQ ID NO:179, and a CDR L3 as set forth in SEQ ID NO:180.

In embodiments, the antibody has a heavy chain variable domain and a light chain variable domain, wherein the heavy chain variable domain includes: a CDR H1 as set forth in SEQ ID NO:151, a CDR H2 as set forth in SEQ ID NO:152 and a CDR H3 as set forth in SEQ ID NO:153; and wherein the light chain variable domain includes: a CDR L1 as set forth in SEQ ID NO:181, a CDR L2 as set forth in SEQ ID NO:182, and a CDR L3 as set forth in SEQ ID NO:183.

In embodiments, the antibody has a heavy chain variable domain and a light chain variable domain, wherein the heavy chain variable domain includes: a CDR H1 as set forth in SEQ ID NO:154, a CDR H2 as set forth in SEQ ID NO:155 and a CDR H3 as set forth in SEQ ID NO:156; and wherein the light chain variable domain includes: a CDR L1 as set forth in SEQ ID NO:184, a CDR L2 as set forth in SEQ ID NO:185, and a CDR L3 as set forth in SEQ ID NO:186.

In embodiments, the antibody has a heavy chain variable domain and a light chain variable domain, wherein the heavy chain variable domain includes: a CDR H1 as set forth in SEQ ID NO:157, a CDR H2 as set forth in SEQ ID NO:158 and a CDR H3 as set forth in SEQ ID NO:159; and wherein the light chain variable domain includes: a CDR L1 as set forth in SEQ ID NO:187, a CDR L2 as set forth in SEQ ID NO:188, and a CDR L3 as set forth in SEQ ID NO:189.

In embodiments, the antibody has a heavy chain variable domain and a light chain variable domain, wherein the heavy chain variable domain includes: a CDR H1 as set forth in SEQ ID NO:160, a CDR H2 as set forth in SEQ ID NO:161 and a CDR H3 as set forth in SEQ ID NO:162; and wherein the light chain variable domain includes: a CDR L1 as set forth in SEQ ID NO:190, a CDR L2 as set forth in SEQ ID NO:191, and a CDR L3 as set forth in SEQ ID NO:192.

In embodiments, the antibody has a heavy chain variable domain and a light chain variable domain, wherein the heavy chain variable domain includes: a CDR H1 as set forth in SEQ ID NO:163, a CDR H2 as set forth in SEQ ID NO:164 and a CDR H3 as set forth in SEQ ID NO:165; and wherein the light chain variable domain includes: a CDR L1 as set forth in SEQ ID NO:193, a CDR L2 as set forth in SEQ ID NO:194, and a CDR L3 as set forth in SEQ ID NO:195.

In embodiments, the antibody has a heavy chain variable domain and a light chain variable domain, wherein the heavy chain variable domain includes: a CDR H1 as set forth in SEQ ID NO:166, a CDR H2 as set forth in SEQ ID NO:167 and a CDR H3 as set forth in SEQ ID NO:168; and wherein the light chain variable domain includes: a CDR L1 as set forth in SEQ ID NO:196, a CDR L2 as set forth in SEQ ID NO:197, and a CDR L3 as set forth in SEQ ID NO:198.

In embodiments, the antibody has a heavy chain variable domain and a light chain variable domain, wherein the heavy chain variable domain includes: a CDR H1 as set forth in SEQ ID NO:199, a CDR H2 as set forth in SEQ ID NO:200 and a CDR H3 as set forth in SEQ ID NO:201; and wherein the light chain variable domain includes: a CDR L1 as set forth in SEQ ID NO:202, a CDR L2 as set forth in SEQ ID NO:203, and a CDR L3 as set forth in SEQ ID NO:204.

In embodiments, the antibody includes any one of the heavy chain sequences set forth by Table 9, 10, 11 or 12. In embodiments, the antibody includes any one of the heavy chain sequences set forth by Table 9. In embodiments, the antibody includes any one of the heavy chain sequences set forth by Table 10. In embodiments, the antibody includes any one of the heavy chain sequences set forth by Table 11. In embodiments, the antibody includes any one of the heavy chain sequences set forth by Table 12. In embodiments, the antibody includes any one of the light chain sequences set forth by Table 9, 10, 11 or 12. In embodiments, the antibody includes any one of the light chain sequences set forth by Table 9. In embodiments, the antibody includes any one of the light chain sequences set forth by Table 10. In embodiments, the antibody includes any one of the light chain sequences set forth by Table 11. In embodiments, the antibody includes any one of the light chain sequences set forth by Table 12.

In embodiments, the antibody includes any one of the heavy chain sequences set forth by Table 9 and any one of the light chain sequences set forth in Table 9. In embodiments, the antibody includes any one of the heavy chain sequences set forth by Table 9 and any one of the light chain sequences set forth in Table 10. In embodiments, the antibody includes any one of the heavy chain sequences set forth by Table 9 and any one of the light chain sequences set forth in Table 11. In embodiments, the antibody includes any one of the heavy chain sequences set forth by Table 9 and any one of the light chain sequences set forth in Table 12. In embodiments, the antibody includes any one of the heavy chain sequences set forth by Table 10 and any one of the light chain sequences set forth in Table 9. In embodiments, the antibody includes any one of the heavy chain sequences set forth by Table and any one of the light chain sequences set forth in Table 10. In embodiments, the 10 antibody includes any one of the heavy chain sequences set forth by Table 10 and any one of the light chain sequences set forth in Table 11. In embodiments, the antibody includes any one of the heavy chain sequences set forth by Table 10 and any one of the light chain sequences set forth in Table 12. In embodiments, the antibody includes any one of the heavy chain sequences set forth by Table 11 and any one of the light chain sequences set forth in Table 9. In embodiments, the antibody includes any one of the heavy chain sequences set forth by Table 11 and any one of the light chain sequences set forth in Table 10. In embodiments, the antibody includes any one of the heavy chain sequences set forth by Table 11 and any one of the light chain sequences set forth in Table 11. In embodiments, the antibody includes any one of the heavy chain sequences set forth by Table 11 and any one of the light chain sequences set forth in Table 12. In embodiments, the antibody includes any one of the heavy chain sequences set forth by Table 12 and any one of the light chain sequences set forth in Table 9. In embodiments, the antibody includes any one of the heavy chain sequences set forth by Table 12 and any one of the light chain sequences set forth in Table 10. In embodiments, the antibody includes any one of the heavy chain sequences set forth by Table 12 and any one of the light chain sequences set forth in Table 11. In embodiments, the antibody includes any one of the heavy chain sequences set forth by Table 12 and any one of the light chain sequences set forth in Table 12.

In embodiments, the antibody includes any one of the heavy chain sequence and light chain sequence combinations set forth by Table 9, 10, 11 or 12. In embodiments, the antibody has a heavy chain sequence of SEQ ID NO:205 and a light sequence of SEQ ID NO:206. In embodiments, the antibody has a heavy chain sequence of SEQ ID NO:207 and a light sequence of SEQ ID NO:208. In embodiments, the antibody has a heavy chain sequence of SEQ ID NO:209 and a light sequence of SEQ ID NO:210. In embodiments, the antibody has a heavy chain sequence of SEQ ID NO:211 and a light sequence of SEQ ID NO:212. In embodiments, the antibody has a heavy chain sequence of SEQ ID NO:213 and a light sequence of SEQ ID NO:214. In embodiments, the antibody has a heavy chain sequence of SEQ ID NO:215 and a light sequence of SEQ ID NO:216. In embodiments, the antibody has a heavy chain sequence of SEQ ID NO:217 and a light sequence of SEQ ID NO:218. In embodiments, the antibody has a heavy chain sequence of SEQ ID NO:219 and a light sequence of SEQ ID NO:220. In embodiments, the antibody has a heavy chain sequence of SEQ ID NO:221 and a light sequence of SEQ ID NO:222. In embodiments, the antibody has a heavy chain sequence of SEQ ID NO:223 and a light sequence of SEQ ID NO:224. In embodiments, the antibody has a heavy chain sequence of SEQ ID NO:225 and a light sequence of SEQ ID NO:226. In embodiments, the antibody has a heavy chain sequence of SEQ ID NO:227 and a light sequence of SEQ ID NO:228. In embodiments, the antibody has a heavy chain sequence of SEQ ID NO:229 and a light sequence of SEQ ID NO:230. In embodiments, the antibody has a heavy chain sequence of SEQ ID NO:231 and a light sequence of SEQ ID NO:232. In embodiments, the antibody has a heavy chain sequence of SEQ ID NO:233 and a light sequence of SEQ ID NO:234. In embodiments, the antibody has a heavy chain sequence of SEQ ID NO:235 and a light sequence of SEQ ID NO:236. In embodiments, the antibody has a heavy chain sequence of SEQ ID NO:237 and a light sequence of SEQ ID NO:238. In embodiments, the antibody has a heavy chain sequence of SEQ ID NO:239 and a light sequence of SEQ ID NO:240. In embodiments, the 20) antibody has a heavy chain sequence of SEQ ID NO:241 and a light sequence of SEQ ID NO:242. In embodiments, the antibody has a heavy chain sequence of SEQ ID NO:243 and a light sequence of SEQ ID NO:244. In embodiments, the antibody has a heavy chain sequence of SEQ ID NO:245 and a light sequence of SEQ ID NO:246. In embodiments, the antibody has a heavy chain sequence of SEQ ID NO:247 and a light sequence of SEQ ID NO:248. In embodiments, the antibody has a heavy chain sequence of SEQ ID NO:249 and a light sequence of SEQ ID NO:250. In embodiments, the antibody has a heavy chain sequence of SEQ ID NO:251 and a light sequence of SEQ ID NO:252. In embodiments, the antibody has a heavy chain sequence of SEQ ID NO:253 and a light sequence of SEQ ID NO:254. In embodiments, the antibody has a heavy chain sequence of SEQ ID NO:255 and a light sequence of SEQ ID NO:256. In embodiments, the antibody has a heavy chain sequence of SEQ ID NO:257 and a light sequence of SEQ ID NO:258. In embodiments, the antibody has a heavy chain sequence of SEQ ID NO:259 and a light sequence of SEQ ID NO:260. In embodiments, the antibody has a heavy chain sequence of SEQ ID NO:261 and a light sequence of SEQ ID NO:262. In embodiments, the antibody has a heavy chain sequence of SEQ ID NO:263 and a light sequence of SEQ ID NO:264. In embodiments, the antibody has a heavy chain sequence of SEQ ID NO:265 and a light sequence of SEQ ID NO:266. In embodiments, the antibody has a heavy chain sequence of SEQ ID NO:267 and a light sequence of SEQ ID NO:268. In embodiments, the antibody has a heavy chain sequence of SEQ ID NO:269 and a light sequence of SEQ ID NO:270. In embodiments, the antibody has a heavy chain sequence of SEQ ID NO:271 and a light sequence of SEQ ID NO:272.

In embodiments, the antibody is capable of binding to EGFR. In embodiments, the antibody is capable of binding domain IV of EGFR. In embodiments, the antibody does not substantially bind to domain I, domain II or domain III of EGFR. In embodiments, the antibody does not substantially bind to domain I of EGFR. In embodiments, the antibody does not substantially bind to domain II of EGFR. In embodiments, the antibody does not substantially bind to domain III of EGFR. An antibody does not substantially bind to a domain of EGFR wherein using conventional methods and compositions well known and used in the art to detect the interaction of an antibody to an epitope (e.g., immunofluorescence, Western Blot analysis, FACS analysis) do not reveal a detectable level of binding relative to a standard control (e.g., an antibody known in the art to bind to domain III of EGFR). In embodiments, the antibody binds a truncated domain IV EGFR and does not substantially bind to EGFR comprising domain I, domain II, domain II and domain IV.

The antibody provided herein including embodiments thereof may be a humanized antibody, a Fab′ fragment, a single chain antibody (scFv) or a chimeric antibody. Thus, in embodiments, the antibody is a humanized antibody. In embodiments, antibody is a Fab′ fragment. In embodiments, the antibody is a scFv. In embodiments, the antibody is a chimeric antibody.

In embodiments, the antibody includes a fragment crystallizable (Fc) domain. In embodiments, the Fc domain binds an effector cell ligand. The term “effector cell ligand” as provided herein refers to a cell surface molecule expressed on an effector cell of the immune system (e.g., a cytotoxic T cell, a helper T cell, a B cell, a natural killer cell). Upon binding of the antibody to the effector cell ligand expressed on the effector cell, the effector cell is activated and able to exert its function (e.g., selective killing or eradication of malignant, infected or otherwise unhealthy cells). In embodiments, the effector cell ligand is a CD3 protein. In embodiments, the effector cell ligand is a CD16 protein. In embodiments, the CD16 protein includes a valine at a position corresponding to the position of amino acid residue 158. In embodiments, the CD16 protein includes a phenylalanine at a position corresponding to the position of amino acid residue 158. In embodiments, the effector cell ligand is a CD32 protein. In embodiments, the effector cell ligand is a NKp46 protein.

In embodiments, the Fc domain includes an effector cell inhibiting substitution. In the presence of an effector cell inhibiting substitution the binding of the Fc domain to the effector cell ligand decreases activation of an effector cell relative to the absence of said substitution. In embodiments, the binding of the Fc domain to the effector cell ligand results in substantially no activation of an effector cell relative to the absence of said substitution. In embodiments, the Fc domain includes a N297G substitution, a R292C substitution, a V302C substitution or a combination thereof. In embodiments, the Fc domain includes a N297G substitution. In embodiments, the Fc domain includes a R292C substitution. In embodiments, the Fc domain includes a V302C substitution. Thus, in embodiments, the effector cell inhibiting substitution is a N297G substitution, a R292C substitution, or a V302C substitution.

In embodiments, the Fc domain includes an effector cell enhancing substitution. In the presence of an effector cell enhancing substitution the binding of the Fc domain to the effector cell ligand increases activation of an effector cell relative to the absence of said substitution. In embodiments, the Fc domain includes a S239D substitution, a 1332E substitution or a combination thereof. In embodiments, the Fc domain includes a S239D substitution. In embodiments, the Fc domain includes a 1332E substitution. Thus, in embodiments, the effector cell enhancing substitution is a S239D substitution, or a 1332E substitution.

In embodiments, the antibody provided herein is an IgG. In embodiments, the antibody is a human IgG1. In embodiments, the antibody is capable of eliciting antibody-dependent cell mediated cytotoxicity (ADCC). In embodiments, the antibody is a human IgG2. In embodiments, the antibody is a human IgG2 and the antibody does not elicit ADCC. Wherein the antibody does not elicit ADCC, ADCC is not elicited in a detectable amount. In embodiments, the antibody does not elicit target cell killing in the presence of an effector cell at a detectable amount. Standard methods and compositions conventional in the biological arts are contemplated for detecting ADCC using the antibodies provided herein.

The Fab′ domain of an antibody provided herein including embodiments thereof may bind its epitope (i.e. EGFR) with a binding affinity that is different relative to the binding affinity of the IgG isotype of that same antibody. In embodiments, the Fab domain of the anti-EGFR antibody binds EGFR with a greater K_{D} relative to said IgG. In embodiments, a monovalent form of the antibody binds EGFR with a greater equilibrium dissociation constant (K_{D}) relative to a bivalent form of the antibody. In embodiments, the monovalent form binds EGFR with about 100- to 1000-fold greater K_{D} relative to said bivalent form. In embodiments, the monovalent form binds EGFR with about 200- to 400-fold greater K_{D} relative to said bivalent form.

In embodiments, the Fab′ fragment binds EGFR with a greater equilibrium dissociation constant (K_{D}) relative to the IgG (e.g., human IgG1 or human IgG2). In embodiments, the Fab′ fragment binds EGFR with about 100- to 1000-fold greater K_{D} relative to the IgG. In embodiments, the Fab′ fragment binds EGFR with about 150- to 1000-fold greater K_{D} relative to the IgG. In embodiments, the Fab′ fragment binds EGFR with about 200- to 1000-fold greater K_{D} relative to the IgG. In embodiments, the Fab′ fragment binds EGFR with about 250- to 1000-fold greater K_{D} relative to the IgG. In embodiments, the Fab′ fragment binds EGFR with about 300- to 1000-fold greater K_{D} relative to the IgG. In embodiments, the Fab′ fragment binds EGFR with about 350- to 1000-fold greater K_{D} relative to the IgG. In embodiments, the Fab′ fragment binds EGFR with about 400- to 1000-fold greater K_{D} relative to the IgG. In embodiments, the Fab′ fragment binds EGFR with about 450- to 1000-fold greater K_{D} relative to the IgG. In embodiments, the Fab′ fragment binds EGFR with about 500- to 1000-fold greater K_{D} relative to the IgG. In embodiments, the Fab′ fragment binds EGFR with about 550- to 1000-fold greater K_{D} relative to the IgG. In embodiments, the Fab′ fragment binds EGFR with about 600- to 1000-fold greater K_{D} relative to the IgG. In embodiments, the Fab′ fragment binds EGFR with about 650- to 1000-fold greater K_{D} relative to the IgG. In embodiments, the Fab′ fragment binds EGFR with about 700- to 1000-fold greater K_{D} relative to the IgG. In embodiments, the Fab′ fragment binds EGFR with about 750- to 1000-fold greater K_{D} relative to the IgG. In embodiments, the Fab′ fragment binds EGFR with about 800- to 1000-fold greater K_{D} relative to the IgG. In embodiments, the Fab′ fragment binds EGFR with about 850- to 1000-fold greater K_{D} relative to the IgG. In embodiments, the Fab′ fragment binds EGFR with about 900- to 1000-fold greater K_{D} relative to the IgG. In embodiments, the Fab′ fragment binds EGFR with about 950- to 1000-fold greater K_{D} relative to the IgG.

In embodiments, the Fab′ fragment binds EGFR with about 100- to 950-fold greater K_{D} relative to the IgG. In embodiments, the Fab′ fragment binds EGFR with about 100- to 900-fold greater K_{D} relative to the IgG. In embodiments, the Fab′ fragment binds EGFR with about 100- to 850-fold greater K_{D} relative to the IgG. In embodiments, the Fab′ fragment binds EGFR with about 100- to 800-fold greater K_{D} relative to the IgG. In embodiments, the Fab′ fragment binds EGFR with about 100- to 750-fold greater K_{D} relative to the IgG. In embodiments, the Fab′ fragment binds EGFR with about 100- to 700-fold greater K_{D} relative to the IgG. In embodiments, the Fab′ fragment binds EGFR with about 100- to 650-fold greater K_{D} relative to the IgG. In embodiments, the Fab′ fragment binds EGFR with about 100- to 600-fold greater K_{D} relative to the IgG. In embodiments, the Fab′ fragment binds EGFR with about 100- to 550-fold greater K_{D} relative to the IgG. In embodiments, the Fab′ fragment binds EGFR with about 100- to 500-fold greater K_{D} relative to the IgG. In embodiments, the Fab′ fragment binds EGFR with about 100- to 450-fold greater K_{D} relative to the IgG. In embodiments, the Fab′ fragment binds EGFR with about 100- to 400-fold greater K_{D} relative to the IgG. In embodiments, the Fab′ fragment binds EGFR with about 100- to 350-fold greater K_{D} relative to the IgG. In embodiments, the Fab′ fragment binds EGFR with about 100- to 300-fold greater K_{D} relative to the IgG. In embodiments, the Fab′ fragment binds EGFR with about 100- to 350-fold greater K_{D} relative to the IgG. In embodiments, the Fab′ fragment binds EGFR with about 100- to 200-fold greater K_{D} relative to the IgG. In embodiments, the Fab′ fragment binds EGFR with about 100- to 150-fold greater K_{D} relative to the IgG.

In embodiments, the Fab′ fragment binds EGFR with about 100-, 150-, 200-, 250-, 300-, 350-, 400-, 450-, 500-, 550-, 600-, 650-, 700-, 750-, 800-, 850-, 900-, 950-, or 1000-fold greater K_{D} relative to the IgG.

In embodiments, the Fab′ fragment binds EGFR with 100- to 1000-fold greater K_{D} relative to the IgG. In embodiments, the Fab′ fragment binds EGFR with 150- to 1000-fold greater K_{D} relative to the IgG. In embodiments, the Fab′ fragment binds EGFR with 200- to 1000-fold greater K_{D} relative to the IgG. In embodiments, the Fab′ fragment binds EGFR with 250- to 1000-fold greater K_{D} relative to the IgG. In embodiments, the Fab′ fragment binds EGFR with 300- to 1000-fold greater K_{D} relative to the IgG. In embodiments, the Fab fragment binds EGFR with 350- to 1000-fold greater K_{D} relative to the IgG. In embodiments, the Fab′ fragment binds EGFR with 400- to 1000-fold greater K_{D} relative to the IgG. In embodiments, the Fab′ fragment binds EGFR with 450- to 1000-fold greater K_{D} relative to the IgG. In embodiments, the Fab′ fragment binds EGFR with 500- to 1000-fold greater K_{D} relative to the IgG. In embodiments, the Fab′ fragment binds EGFR with 550- to 1000-fold greater K_{D} relative to the IgG. In embodiments, the Fab′ fragment binds EGFR with 600- to 1000-fold greater K_{D} relative to the IgG. In embodiments, the Fab′ fragment binds EGFR with 650- to 1000-fold greater K_{D} relative to the IgG. In embodiments, the Fab′ fragment binds EGFR with 700- to 1000-fold greater K_{D} relative to the IgG. In embodiments, the Fab′ fragment binds EGFR with 750- to 1000-fold greater K_{D} relative to the IgG. In embodiments, the Fab′ fragment binds EGFR with 800- to 1000-fold greater K_{D} relative to the IgG. In embodiments, the Fab′ fragment binds EGFR with 850- to 1000-fold greater K_{D} relative to the IgG. In embodiments, the Fab′ fragment binds EGFR with 900- to 1000-fold greater K_{D} relative to the IgG. In embodiments, the Fab′ fragment binds EGFR with 950- to 1000-fold greater K_{D} relative to the IgG.

in embodiments, the Fab′ fragment binds EGFR with 100- to 950-fold greater K_{D} relative to the IgG. In embodiments, the Fab′ fragment binds EGFR with 100- to 900-fold greater K_{D} relative to the IgG. In embodiments, the Fab′ fragment binds EGFR with 100- to 850-fold greater K_{D} relative to the IgG. In embodiments, the Fab′ fragment binds EGFR with 100- to 800-fold greater K_{D} relative to the IgG. In embodiments, the Fab′ fragment binds EGFR with 100- to 750-fold greater K_{D} relative to the IgG. In embodiments, the Fab fragment binds EGFR with 100- to 700-fold greater K_{D} relative to the IgG. In embodiments, the Fab′ fragment binds EGFR with 100- to 650-fold greater K_{D} relative to the IgG. In embodiments, the Fab′ fragment binds EGFR with 100- to 600-fold greater K_{D} relative to the IgG. In embodiments, the Fab′ fragment binds EGFR with 100- to 550-fold greater K_{D} relative to the IgG. In embodiments, the Fab′ fragment binds EGFR with 100- to 500-fold greater K_{D} relative to the IgG. In embodiments, the Fab′ fragment binds EGFR with 100- to 450-fold greater K_{D} relative to the IgG. In embodiments, the Fab′ fragment binds EGFR with 100- to 400-fold greater K_{D} relative to the IgG. In embodiments, the Fab′ fragment binds EGFR with 100- to 350-fold greater K_{D} relative to the IgG. In embodiments, the Fab′ fragment binds EGFR with 100- to 300-fold greater K_{D} relative to the IgG. In embodiments, the Fab′ fragment binds EGFR with 100- to 350-fold greater K_{D} relative to the IgG. In embodiments, the Fab′ fragment binds EGFR with 100- to 200-fold greater K_{D} relative to the IgG. In embodiments, the Fab′ fragment binds EGFR with 100- to 150-fold greater K_{D} relative to the IgG.

In embodiments, the Fab′ fragment binds EGFR with 100-, 150-, 200-, 250-, 300-, 350-, 400-, 450-, 500-, 550-, 600-, 650-, 700-, 750-, 800-, 850-, 900-, 950-, or 1000-fold greater K_{D} relative to the IgG.

In embodiments, the Fab′ fragment binds EGFR with about 200- to 400-fold greater K_{D} relative to the IgG. In embodiments, the Fab′ fragment binds EGFR with about 210- to 400-fold greater K_{D} relative to the IgG. In embodiments, the Fab′ fragment binds EGFR with about 220- to 400-fold greater K_{D} relative to the IgG. In embodiments, the Fab′ fragment binds EGFR with about 230- to 400-fold greater K_{D} relative to the IgG. In embodiments, the Fab′ fragment binds EGFR with about 240- to 400-fold greater K_{D} relative to the IgG. In embodiments, the Fab′ fragment binds EGFR with about 250- to 400-fold greater K_{D} relative to the IgG. In embodiments, the Fab′ fragment binds EGFR with about 260- to 400-fold greater K_{D} relative to the IgG. In embodiments, the Fab′ fragment binds EGFR with about 270- to 400-fold greater K_{D} relative to the IgG. In embodiments, the Fab′ fragment binds EGFR with about 280- to 400-fold greater K_{D} relative to the IgG. In embodiments, the Fab′ fragment binds EGFR with about 290- to 400-fold greater K_{D} relative to the IgG. In embodiments, the Fab′ fragment binds EGFR with about 300- to 400-fold greater K_{D} relative to the IgG. In embodiments, the Fab′ fragment binds EGFR with about 310- to 400-fold greater K_{D} relative to the IgG. In embodiments, the Fab′ fragment binds EGFR with about 320)- to 400-fold greater K_{D} relative to the IgG. In embodiments, the Fab′ fragment binds EGFR with about 330- to 400-fold greater K_{D} relative to the IgG. In embodiments, the Fab′ fragment binds EGFR with about 340- to 400-fold greater K_{D} relative to the IgG. In embodiments, the Fab′ fragment binds EGFR with about 350- to 400-fold greater K_{D} relative to the IgG. In embodiments, the Fab′ fragment binds EGFR with about 360- to 400-fold greater K_{D} relative to the IgG. In embodiments, the Fab′ fragment binds EGFR with about 370- to 400-fold greater K_{D} relative to the IgG. In embodiments, the Fab′ fragment binds EGFR with about 380- to 400-fold greater K_{D} relative to the IgG. In embodiments, the Fab′ fragment binds EGFR with about 390- to 400-fold greater K_{D} relative to the IgG.

In embodiments, the Fab′ fragment binds EGFR with about 200- to 390-fold greater K_{D} relative to the IgG. In embodiments, the Fab′ fragment binds EGFR with about 200- to 380-fold greater K_{D} relative to the IgG. In embodiments, the Fab′ fragment binds EGFR with about 200- to 370-fold greater K_{D} relative to the IgG. In embodiments, the Fab′ fragment binds EGFR with about 200- to 360-fold greater K_{D} relative to the IgG. In embodiments, the Fab′ fragment binds EGFR with about 200- to 350-fold greater K_{D} relative to the IgG. In embodiments, the Fab′ fragment binds EGFR with about 200- to 340-fold greater K_{D} relative to the IgG. In embodiments, the Fab′ fragment binds EGFR with about 200- to 330-fold greater K_{D} relative to the IgG. In embodiments, the Fab′ fragment binds EGFR with about 200- to 320-fold greater K_{D} relative to the IgG. In embodiments, the Fab′ fragment binds EGFR with about 200- to 310-fold greater K_{D} relative to the IgG. In embodiments, the Fab′ fragment binds EGFR with about 200- to 300-fold greater K_{D} relative to the IgG. In embodiments, the Fab′ fragment binds EGFR with about 200- to 290-fold greater K_{D} relative to the IgG. In embodiments, the Fab′ fragment binds EGFR with about 200- to 280-fold greater K_{D} relative to the IgG. In embodiments, the Fab′ fragment binds EGFR with about 200- to 270-fold greater K_{D} relative to the IgG. In embodiments, the Fab′ fragment binds EGFR with about 200- to 260-fold greater K_{D} relative to the IgG. In embodiments, the Fab′ fragment binds EGFR with about 200- to 250-fold greater K_{D} relative to the IgG. In embodiments, the Fab′ fragment binds EGFR with about 200- to 240-fold greater K_{D} relative to the IgG. In embodiments, the Fab′ fragment binds EGFR with about 200- to 230-fold greater K_{D} relative to the IgG. In embodiments, the Fab′ fragment binds EGFR with about 200- to 220-fold greater K_{D} relative to the IgG. In embodiments, the Fab′ fragment binds EGFR with about 200- to 210-fold greater K_{D} relative to the IgG.

In embodiments, the Fab′ fragment binds EGFR with about 200-, 210-, 220-, 230-, 240-, 250-, 260-, 270-, 280-, 290-, 300-, 310-, 320-, 330-, 340-, 340-, 350-, 370-, 380-, 390- or 400-fold greater K_{D} relative to the IgG.

In embodiments, the Fab′ fragment binds EGFR with 200- to 400-fold greater K_{D} relative to the IgG. In embodiments, the Fab′ fragment binds EGFR with 210- to 400-fold greater K_{D} relative to the IgG. In embodiments, the Fab′ fragment binds EGFR with 220- to 400-fold greater K_{D} relative to the IgG. In embodiments, the Fab′ fragment binds EGFR with 230- to 400-fold greater K_{D} relative to the IgG. In embodiments, the Fab′ fragment binds EGFR with 240- to 400-fold greater K_{D} relative to the IgG. In embodiments, the Fab′ fragment binds EGFR with 250- to 400-fold greater K_{D} relative to the IgG. In embodiments, the Fab′ fragment binds EGFR with 260- to 400-fold greater K_{D} relative to the IgG. In embodiments, the Fab′ fragment binds EGFR with 270- to 400-fold greater K_{D} relative to the IgG. In embodiments, the Fab′ fragment binds EGFR with 280- to 400-fold greater K_{D} relative to the IgG. In embodiments, the Fab′ fragment binds EGFR with 290- to 400-fold greater K_{D} relative to the IgG. In embodiments, the Fab′ fragment binds EGFR with 300- to 400-fold greater K_{D} relative to the IgG. In embodiments, the Fab′ fragment binds EGFR with 310- to 400-fold greater K_{D} relative to the IgG. In embodiments, the Fab′ fragment binds EGFR with 320- to 400-fold greater K_{D} relative to the IgG. In embodiments, the Fab fragment binds EGFR with 330- to 400-fold greater K_{D} relative to the IgG. In embodiments, the Fab′ fragment binds EGFR with 340- to 400-fold greater K_{D} relative to the IgG. In embodiments, the Fab′ fragment binds EGFR with 350- to 400-fold greater K_{D} relative to the IgG. In embodiments, the Fab′ fragment binds EGFR with 360- to 400-fold greater K_{D} relative to the IgG. In embodiments, the Fab′ fragment binds EGFR with 370- to 400-fold greater K_{D} relative to the IgG. In embodiments, the Fab′ fragment binds EGFR with 380- to 400-fold greater K_{D} relative to the IgG. In embodiments, the Fab′ fragment binds EGFR with 390- to 400-fold greater K_{D} relative to the IgG.

In embodiments, the Fab′ fragment binds EGFR with 200- to 390-fold greater K_{D} relative to the IgG. In embodiments, the Fab′ fragment binds EGFR with 200- to 380-fold greater K_{D} relative to the IgG. In embodiments, the Fab′ fragment binds EGFR with 200- to 370-fold greater K_{D} relative to the IgG. In embodiments, the Fab′ fragment binds EGFR with 200- to 360-fold greater K_{D} relative to the IgG. In embodiments, the Fab′ fragment binds EGFR with 200- to 350-fold greater K_{D} relative to the IgG. In embodiments, the Fab′ fragment binds EGFR with 200- to 340-fold greater K_{D} relative to the IgG. In embodiments, the Fab′ fragment binds EGFR with 200- to 330-fold greater K_{D} relative to the IgG. In embodiments, the Fab′ fragment binds EGFR with 200- to 320-fold greater K_{D} relative to the IgG. In embodiments, the Fab′ fragment binds EGFR with 200- to 310-fold greater K_{D} relative to the IgG. In embodiments, the Fab′ fragment binds EGFR with 200- to 300-fold greater K_{D} relative to the IgG. In embodiments, the Fab′ fragment binds EGFR with 200- to 290-fold greater K_{D} relative to the IgG. In embodiments, the Fab′ fragment binds EGFR with 200- to 280-fold greater K_{D} relative to the IgG. In embodiments, the Fab′ fragment binds EGFR with 200- to 270-fold greater K_{D} relative to the IgG. In embodiments, the Fab fragment binds EGFR with 200- to 260-fold greater K_{D} relative to the IgG. In embodiments, the Fab′ fragment binds EGFR with 200- to 250-fold greater K_{D} relative to the IgG. In embodiments, the Fab′ fragment binds EGFR with 200- to 240-fold greater K_{D} relative to the IgG. In embodiments, the Fab′ fragment binds EGFR with 200- to 230-fold greater K_{D} relative to the IgG. In embodiments, the Fab′ fragment binds EGFR with 200- to 220-fold greater K_{D} relative to the IgG. In embodiments, the Fab′ fragment binds EGFR with 200- to 210-fold greater K_{D} relative to the IgG.

In embodiments, the Fab′ fragment binds EGFR with 200-, 210-, 220-, 230-, 240-, 250-, 260-, 270-, 280-, 290-, 300-, 310-, 320-, 330-, 340-, 340-, 350-, 370-, 380-, 390- or 400-fold greater K_{D} relative to the IgG.

In embodiments, the Fab′ fragment binds EGFR with a K_{D} of about 100 nM to about 500 nM. In embodiments, the Fab′ fragment binds EGFR with a K_{D} of about 120 nM to about 500 nM. In embodiments, the Fab′ fragment binds EGFR with a K_{D} of about 140 nM to about 500 nM. In embodiments, the Fab′ fragment binds EGFR with a K_{D} of about 160 nM to about 500 nM. In embodiments, the Fab′ fragment binds EGFR with a K_{D} of about 180 nM to about 500 nM. In embodiments, the Fab′ fragment binds EGFR with a K_{D} of about 200 nM to about 500 nM. In embodiments, the Fab′ fragment binds EGFR with a K_{D} of about 220 nM to about 500 nM. In embodiments, the Fab′ fragment binds EGFR with a K_{D} of about 240 nM to about 500 nM. In embodiments, the Fab′ fragment binds EGFR with a K_{D} of about 260 nM to about 500 nM. In embodiments, the Fab′ fragment binds EGFR with a K_{D} of about 280 nM to about 500 nM. In embodiments, the Fab′ fragment binds EGFR with a K_{D} of about 300 nM to about 500 nM. In embodiments, the Fab′ fragment binds EGFR with a K_{D} of about 320 nM to about 500 nM. In embodiments, the Fab fragment binds EGFR with a K_{D} of about 340 nM to about 500 nM. In embodiments, the Fab′ fragment binds EGFR with a K_{D} of about 360 nM to about 500 nM. In embodiments, the Fab′ fragment binds EGFR with a K_{D} of about 380 nM to about 500 nM. In embodiments, the Fab′ fragment binds EGFR with a K_{D} of about 400 nM to about 500 nM. In embodiments, the Fab′ fragment binds EGFR with a K_{D} of about 420 nM to about 500 nM. In embodiments, the Fab′ fragment binds EGFR with a K_{D} of about 440 nM to about 500 nM. In embodiments, the Fab′ fragment binds EGFR with a K_{D} of about 460 nM to about 500 nM. In embodiments, the Fab′ fragment binds EGFR with a K_{D} of about 480 nM to about 500 nM.

In embodiments, the Fab′ fragment binds EGFR with a K_{D} of about 100 nM to about 480 nM. In embodiments, the Fab′ fragment binds EGFR with a K_{D} of about 100 nM to about 460 nM. In embodiments, the Fab′ fragment binds EGFR with a K_{D} of about 100 nM to about 440) nM. In embodiments, the Fab′ fragment binds EGFR with a K_{D} of about 100) nM to about 420 nM. In embodiments, the Fab′ fragment binds EGFR with a K_{D} of about 100 nM to about 400 nM. In embodiments, the Fab′ fragment binds EGFR with a K_{D} of about 100 nM to about 380) nM. In embodiments, the Fab′ fragment binds EGFR with a K_{D} of about 100 nM to about 360 nM. In embodiments, the Fab′ fragment binds EGFR with a K_{D} of about 100 nM to about 340 nM. In embodiments, the Fab′ fragment binds EGFR with a K_{D} of about 100 nM to about 320 nM. In embodiments, the Fab′ fragment binds EGFR with a K_{D} of about 100 nM to about 300 nM. In embodiments, the Fab′ fragment binds EGFR with a K_{D} of about 100 nM to about 280) nM. In embodiments, the Fab′ fragment binds EGFR with a K_{D} of about 100 nM to about 260 nM. In embodiments, the Fab fragment binds EGFR with a K_{D} of about 100 nM to about 240) nM. In embodiments, the Fab′ fragment binds EGFR with a K_{D} of about 100 nM to about 220 nM. In embodiments, the Fab′ fragment binds EGFR with a K_{D} of about 100 nM to about 200 nM. In embodiments, the Fab′ fragment binds EGFR with a K_{D} of about 100 nM to about 180 nM. In embodiments, the Fab′ fragment binds EGFR with a K_{D} of about 100 nM to about 160 nM. In embodiments, the Fab′ fragment binds EGFR with a K_{D} of about 100 nM to about 140 nM. In embodiments, the Fab′ fragment binds EGFR with a K_{D} of about 100 nM to about 120 nM.

In embodiments, the Fab′ fragment binds EGFR with a K_{D} of about 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440) nM, 460 nM, 480 nM, or 500 nM.

In embodiments, the Fab′ fragment binds EGFR with a K_{D} of 100 nM to 500 nM. In embodiments, the Fab′ fragment binds EGFR with a K_{D} of 120 nM to 500 nM. In embodiments, the Fab′ fragment binds EGFR with a K_{D} of 140) nM to 500 nM. In embodiments, the Fab′ fragment binds EGFR with a K_{D} of 160 nM to 500 nM. In embodiments, the Fab′ fragment binds EGFR with a K_{D} of 180) nM to 500 nM. In embodiments, the Fab′ fragment binds EGFR with a K_{D} of 200 nM to 500 nM. In embodiments, the Fab′ fragment binds EGFR with a K_{D} of 220 nM to 500 nM. In embodiments, the Fab′ fragment binds EGFR with a K_{D} of 240 nM to 500 nM. In embodiments, the Fab′ fragment binds EGFR with a K_{D} of 260 nM to 500 nM. In embodiments, the Fab′ fragment binds EGFR with a K_{D} of 280 nM to 500 nM. In embodiments, the Fab′ fragment binds EGFR with a K_{D} of 300 nM to 500 nM. In embodiments, the Fab′ fragment binds EGFR with a K_{D} of 320 nM to 500 nM. In embodiments, the Fab′ fragment binds EGFR with a K_{D} of 340 nM to 500 nM. In embodiments, the Fab′ fragment binds EGFR with a K_{D} of 360 nM to 500 nM. In embodiments, the Fab′ fragment binds EGFR with a K_{D} of 380 nM to 500 nM. In embodiments, the Fab′ fragment binds EGFR with a K_{D} of 400 nM to 500 nM. In embodiments, the Fab′ fragment binds EGFR with a K_{D} of 420 nM to 500 nM. In embodiments, the Fab′ fragment binds EGFR with a K_{D} of 440 nM to 500 nM. In embodiments, the Fab′ fragment binds EGFR with a K_{D} of 460 nM to 500 nM. In embodiments, the Fab′ fragment binds EGFR with a K_{D} of 480 nM to 500 nM.

In embodiments, the Fab′ fragment binds EGFR with a K_{D} of 100 nM to 480 nM. In embodiments, the Fab′ fragment binds EGFR with a K_{D} of 100 nM to 460 nM. In embodiments, the Fab′ fragment binds EGFR with a K_{D} of 100 nM to 440 nM. In embodiments, the Fab′ fragment binds EGFR with a K_{D} of 100 nM to 420 nM. In embodiments, the Fab′ fragment binds EGFR with a K_{D} of 100 nM to 400 nM. In embodiments, the Fab′ fragment binds EGFR with a K_{D} of 100 nM to 380 nM. In embodiments, the Fab′ fragment binds EGFR with a K_{D} of 100 nM to 360 nM. In embodiments, the Fab′ fragment binds EGFR with a K_{D} of 100 nM to 340 nM. In embodiments, the Fab′ fragment binds EGFR with a K_{D} of 100 nM to 320 nM. In embodiments, the Fab′ fragment binds EGFR with a K_{D} of 100 nM to 300 nM. In embodiments, the Fab′ fragment binds EGFR with a K_{D} of 100 nM to 280 nM. In embodiments, the Fab′ fragment binds EGFR with a K_{D} of 100 nM to 260 nM. In embodiments, the Fab′ fragment binds EGFR with a K_{D} of 100 nM to 240 nM. In embodiments, the Fab′ fragment binds EGFR with a K_{D} of 100 nM to 220 nM. In embodiments, the Fab′ fragment binds EGFR with a K_{D} of 100 nM to 200 nM. In embodiments, the Fab′ fragment binds EGFR with a K_{D} of 100 nM to 180 nM. In embodiments, the Fab′ fragment binds EGFR with a K_{D} of 100 nM to 160 nM. In embodiments, the Fab′ fragment binds EGFR with a K_{D} of 100 nM to 140 nM. In embodiments, the Fab′ fragment binds EGFR with a K_{D} of 100 nM to 120 nM.

In embodiments, the Fab′ fragment binds EGFR with a K_{D} of 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, or 500 nM.

In embodiments, the Fab′ fragment binds EGFR with a K_{D} of about 170 nM. In embodiments, the Fab′ fragment binds EGFR with a K_{D} of 170 nM.

In embodiments, the IgG binds EGFR with a K_{D} from about 100 pM to 1000 pM. In embodiments, the IgG binds EGFR with a K_{D} from about 120 pM to 1000 pM. In embodiments, the IgG binds EGFR with a K_{D} from about 140 pM to 1000 pM. In embodiments, the IgG binds EGFR with a K_{D} from about 160 pM to 1000 pM. In embodiments, the IgG binds EGFR with a K_{D} from about 180 pM to 1000 pM. In embodiments, the IgG binds EGFR with a K_{D} from about 200 pM to 1000 pM. In embodiments, the IgG binds EGFR with a K_{D} from about 220 pM to 1000 pM. In embodiments, the IgG binds EGFR with a K_{D} from about 240 pM to 1000 pM. In embodiments, the IgG binds EGFR with a K_{D} from about 260 pM to 1000 pM. In embodiments, the IgG binds EGFR with a K_{D} from about 280 pM to 1000 pM. In embodiments, the IgG binds EGFR with a K_{D} from about 300 pM to 1000 pM. In embodiments, the IgG binds EGFR with a K_{D} from about 320 pM to 1000 pM. In embodiments, the IgG binds EGFR with a K_{D} from about 340 pM to 1000 pM. In embodiments, the IgG binds EGFR with a K_{D} from about 360 pM to 1000 pM. In embodiments, the IgG binds EGFR with a K_{D} from about 380 pM to 1000 pM. In embodiments, the IgG binds EGFR with a K_{D} from about 400 pM to 1000 pM. In embodiments, the IgG binds EGFR with a K_{D} from about 420 pM to 1000 pM. In embodiments, the IgG binds EGFR with a K_{D} from about 440 pM to 1000 pM. In embodiments, the IgG binds EGFR with a K_{D} from about 460 pM to 1000 pM. In embodiments, the IgG binds EGFR with a K_{D} from about 480 pM to 1000 pM. In embodiments, the IgG binds EGFR with a K_{D} from about 500 pM to 1000 pM. In embodiments, the IgG binds EGFR with a K_{D} from about 520 pM to 1000 pM. In embodiments, the IgG binds EGFR with a K_{D} from about 540 pM to 1000 pM. In embodiments, the IgG binds EGFR with a K_{D} from about 560 pM to 1000 pM.

In embodiments, the IgG binds EGFR with a K_{D} from about 580 pM to 1000 pM. In embodiments, the IgG binds EGFR with a K_{D} from about 600 pM to 1000 pM. In embodiments, the IgG binds EGFR with a K_{D} from about 620 pM to 1000 pM. In embodiments, the IgG binds EGFR with a K_{D} from about 640 pM to 1000 pM. In embodiments, the IgG binds EGFR with a K_{D} from about 660 pM to 1000 pM. In embodiments, the IgG binds EGFR with a K_{D} from about 680 pM to 1000 pM. In embodiments, the IgG binds EGFR with a K_{D} from about 700 pM to 1000 pM. In embodiments, the IgG binds EGFR with a K_{D} from about 720 pM to 1000 pM. In embodiments, the IgG binds EGFR with a K_{D} from about 740 pM to 1000 pM. In embodiments, the IgG binds EGFR with a K_{D} from about 760 pM to 1000 pM. In embodiments, the IgG binds EGFR with a K_{D} from about 780 pM to 1000 pM. In embodiments, the IgG binds EGFR with a K_{D} from about 800 pM to 1000 pM. In embodiments, the IgG binds EGFR with a K_{D} from about 820 pM to 1000 pM. In embodiments, the IgG binds EGFR with a K_{D} from about 840 pM to 1000 pM. In embodiments, the IgG binds EGFR with a K_{D} from about 860 pM to 1000 pM. In embodiments, the IgG binds EGFR with a K_{D} from about 880 pM to 1000 pM. In embodiments, the IgG binds EGFR with a K_{D} from about 900 pM to 1000 pM. In embodiments, the IgG binds EGFR with a K_{D} from about 920 pM to 1000 pM. In embodiments, the IgG binds EGFR with a K_{D} from about 940 pM to 1000 pM. In embodiments, the IgG binds EGFR with a K_{D} from about 960 pM to 1000 pM. In embodiments, the IgG binds EGFR with a K_{D} from about 980 pM to 1000 pM.

In embodiments, the IgG binds EGFR with a K_{D} from about 100 pM to 980 pM. In embodiments, the IgG binds EGFR with a K_{D} from about 100 pM to 960 pM. In embodiments, the IgG binds EGFR with a K_{D} from about 100 pM to 940 pM. In embodiments, the IgG binds EGFR with a K_{D} from about 100 pM to 920 pM. In embodiments, the IgG binds EGFR with a K_{D} from about 100 pM to 900 pM. In embodiments, the IgG binds EGFR with a K_{D} from about 100 pM to 880 pM. In embodiments, the IgG binds EGFR with a K_{D} from about 100 pM to 860 pM. In embodiments, the IgG binds EGFR with a K_{D} from about 100 pM to 840 pM. In embodiments, the IgG binds EGFR with a K_{D} from about 100 pM to 820 pM. In embodiments, the IgG binds EGFR with a K_{D} from about 100 pM to 800 pM. In embodiments, the IgG binds EGFR with a K_{D} from about 100 pM to 780 pM. In embodiments, the IgG binds EGFR with a K_{D} from about 100 pM to 760 pM. In embodiments, the IgG binds EGFR with a K_{D} from about 100 pM to 740 pM. In embodiments, the IgG binds EGFR with a K_{D} from about 100 pM to 720 pM. In embodiments, the IgG binds EGFR with a K_{D} from about 100 pM to 700 pM. In embodiments, the IgG binds EGFR with a K_{D} from about 100 pM to 680 pM. In embodiments, the IgG binds EGFR with a K_{D} from about 100 pM to 660 pM. In embodiments, the IgG binds EGFR with a K_{D} from about 100 pM to 640 pM. In embodiments, the IgG binds EGFR with a K_{D} from about 100 pM to 620 pM. In embodiments, the IgG binds EGFR with a K_{D} from about 100 pM to 600 pM. In embodiments, the IgG binds EGFR with a K_{D} from about 100 pM to 580 pM. In embodiments, the IgG binds EGFR with a K_{D} from about 100 pM to 560 pM.

In embodiments, the IgG binds EGFR with a K_{D} from about 100 pM to 540 pM. In embodiments, the IgG binds EGFR with a K_{D} from about 100 pM to 520 pM. In embodiments, the IgG binds EGFR with a K_{D} from about 100 pM to 500 pM. In embodiments, the IgG binds EGFR with a K_{D} from about 100 pM to 480 pM. In embodiments, the IgG binds EGFR with a K_{D} from about 100 pM to 460 pM. In embodiments, the IgG binds EGFR with a K_{D} from about 100 pM to 440 pM. In embodiments, the IgG binds EGFR with a K_{D} from about 100 pM to 420 pM. In embodiments, the IgG binds EGFR with a K_{D} from about 100 pM to 400 pM. In embodiments, the IgG binds EGFR with a K_{D} from about 100 pM to 380 pM. In embodiments, the IgG binds EGFR with a K_{D} from about 100 pM to 360 pM. In embodiments, the IgG binds EGFR with a K_{D} from about 100 pM to 340 pM. In embodiments, the IgG binds EGFR with a K_{D} from about 100 pM to 320 pM. In embodiments, the IgG binds EGFR with a K_{D} from about 100 pM to 300 pM. In embodiments, the IgG binds EGFR with a K_{D} from about 100 pM to 280 pM. In embodiments, the IgG binds EGFR with a K_{D} from about 100 pM to 260 pM. In embodiments, the IgG binds EGFR with a K_{D} from about 100 pM to 240 pM. In embodiments, the IgG binds EGFR with a K_{D} from about 100 pM to 220 pM. In embodiments, the IgG binds EGFR with a K_{D} from about 100 pM to 200 pM. In embodiments, the IgG binds EGFR with a K_{D} from about 100 pM to 180 pM. In embodiments, the IgG binds EGFR with a K_{D} from about 100 pM to 160 pM. In embodiments, the IgG binds EGFR with a K_{D} from about 100 pM to 140 pM. In embodiments, the IgG binds EGFR with a K_{D} from about 100 pM to 120 pM.

In embodiments, the IgG binds EGFR with a K_{D} from 100 pM to 1000 pM. In embodiments, the IgG binds EGFR with a K_{D} from 120 pM to 1000 pM. In embodiments, the IgG binds EGFR with a K_{D} from 140 pM to 1000 pM. In embodiments, the IgG binds EGFR with a K_{D} from 160 pM to 1000 pM. In embodiments, the IgG binds EGFR with a K_{D} from 180 pM to 1000 pM. In embodiments, the IgG binds EGFR with a K_{D} from 200 pM to 1000 pM. In embodiments, the IgG binds EGFR with a K_{D} from 220 pM to 1000 pM. In embodiments, the IgG binds EGFR with a K_{D} from 240 pM to 1000 pM. In embodiments, the IgG binds EGFR with a K_{D} from 260 pM to 1000 pM. In embodiments, the IgG binds EGFR with a K_{D} from 280 pM to 1000 pM. In embodiments, the IgG binds EGFR with a K_{D} from 300 pM to 1000 pM. In embodiments, the IgG binds EGFR with a K_{D} from 320 pM to 1000 pM. In embodiments, the IgG binds EGFR with a K_{D} from 340 pM to 1000 pM. In embodiments, the IgG binds EGFR with a K_{D} from 360 pM to 1000 pM. In embodiments, the IgG binds EGFR with a K_{D} from 380 pM to 1000 pM. In embodiments, the IgG binds EGFR with a K_{D} from 400 pM to 1000 pM. In embodiments, the IgG binds EGFR with a K_{D} from 420 pM to 1000 pM. In embodiments, the IgG binds EGFR with a K_{D} from 440 pM to 1000 pM. In embodiments, the IgG binds EGFR with a K_{D} from 460 pM to 1000 pM. In embodiments, the IgG binds EGFR with a K_{D} from 480 pM to 1000 pM. In embodiments, the IgG binds EGFR with a K_{D} from 500 pM to 1000 pM.

In embodiments, the IgG binds EGFR with a K_{D} from 520 pM to 1000 pM. In embodiments, the IgG binds EGFR with a K_{D} from 540 pM to 1000 pM. In embodiments, the IgG binds EGFR with a K_{D} from 560 pM to 1000 pM. In embodiments, the IgG binds EGFR with a K_{D} from 580 pM to 1000 pM. In embodiments, the IgG binds EGFR with a K_{D} from 600 pM to 1000 pM. In embodiments, the IgG binds EGFR with a K_{D} from 620 pM to 1000 pM. In embodiments, the IgG binds EGFR with a K_{D} from 640 pM to 1000 pM. In embodiments, the IgG binds EGFR with a K_{D} from 660 pM to 1000 pM. In embodiments, the IgG binds EGFR with a K_{D} from 680 pM to 1000 pM. In embodiments, the IgG binds EGFR with a K_{D} from 700 pM to 1000 pM. In embodiments, the IgG binds EGFR with a K_{D} from 720 pM to 1000 pM. In embodiments, the IgG binds EGFR with a K_{D} from 740 pM to 1000 pM. In embodiments, the IgG binds EGFR with a K_{D} from 760 pM to 1000 pM. In embodiments, the IgG binds EGFR with a K_{D} from 780 pM to 1000 pM. In embodiments, the IgG binds EGFR with a K_{D} from 800 pM to 1000 pM. In embodiments, the IgG binds EGFR with a K_{D} from 820 pM to 1000 pM. In embodiments, the IgG binds EGFR with a K_{D} from 840 pM to 1000 pM. In embodiments, the IgG binds EGFR with a K_{D} from 860 pM to 1000 pM. In embodiments, the IgG binds EGFR with a K_{D} from 880 pM to 1000 pM. In embodiments, the IgG binds EGFR with a K_{D} from 900 pM to 1000 pM. In embodiments, the IgG binds EGFR with a K_{D} from 920 pM to 1000 pM. In embodiments, the IgG binds EGFR with a K_{D} from 940 pM to 1000 pM. In embodiments, the IgG binds EGFR with a K_{D} from 960 pM to 1000 pM. In embodiments, the IgG binds EGFR with a K_{D} from 980 pM to 1000 pM.

In embodiments, the IgG binds EGFR with a K_{D} from 100 pM to 980 pM. In embodiments, the IgG binds EGFR with a K_{D} from 100 pM to 960 pM. In embodiments, the IgG binds EGFR with a K_{D} from 100 pM to 940 pM. In embodiments, the IgG binds EGFR with a K_{D} from 100 pM to 920 pM. In embodiments, the IgG binds EGFR with a K_{D} from 100 pM to 900 pM. In embodiments, the IgG binds EGFR with a K_{D} from 100 pM to 880 pM. In embodiments, the IgG binds EGFR with a K_{D} from 100 pM to 860 pM. In embodiments, the IgG binds EGFR with a K_{D} from 100 pM to 840 pM. In embodiments, the IgG binds EGFR with a K_{D} from 100 pM to 820 pM. In embodiments, the IgG binds EGFR with a K_{D} from 100 pM to 800 pM. In embodiments, the IgG binds EGFR with a K_{D} from 100 pM to 780 pM. In embodiments, the IgG binds EGFR with a K_{D} from 100 pM to 760 pM. In embodiments, the IgG binds EGFR with a K_{D} from 100 pM to 740 pM. In embodiments, the IgG binds EGFR with a K_{D} from 100 pM to 720 pM. In embodiments, the IgG binds EGFR with a K_{D} from 100 pM to 700 pM. In embodiments, the IgG binds EGFR with a K_{D} from 100 pM to 680 pM. In embodiments, the IgG binds EGFR with a K_{D} from 100 pM to 660 pM.

In embodiments, the IgG binds EGFR with a K_{D} from 100 pM to 640 pM. In embodiments, the IgG binds EGFR with a K_{D} from 100 pM to 620 pM. In embodiments, the IgG binds EGFR with a K_{D} from 100 pM to 600 pM. In embodiments, the IgG binds EGFR with a K_{D} from 100 pM to 580 pM. In embodiments, the IgG binds EGFR with a K_{D} from 100 pM to 560 pM. In embodiments, the IgG binds EGFR with a K_{D} from 100 pM to 540 pM. In embodiments, the IgG binds EGFR with a K_{D} from 100 pM to 520 pM. In embodiments, the IgG binds EGFR with a K_{D} from 100 pM to 500 pM. In embodiments, the IgG binds EGFR with a K_{D} from 100 pM to 480 pM. In embodiments, the IgG binds EGFR with a K_{D} from 100 pM to 460 pM. In embodiments, the IgG binds EGFR with a K_{D} from 100 pM to 440 pM. In embodiments, the IgG binds EGFR with a K_{D} from 100 pM to 420 pM. In embodiments, the IgG binds EGFR with a K_{D} from 100 pM to 400 pM. In embodiments, the IgG binds EGFR with a K_{D} from 100 pM to 380 pM. In embodiments, the IgG binds EGFR with a K_{D} from 100 pM to 360 pM. In embodiments, the IgG binds EGFR with a K_{D} from 100 pM to 340 pM. In embodiments, the IgG binds EGFR with a K_{D} from 100 pM to 320 pM. In embodiments, the IgG binds EGFR with a K_{D} from 100 pM to 300 pM. In embodiments, the IgG binds EGFR with a K_{D} from 100 pM to 280 pM. In embodiments, the IgG binds EGFR with a K_{D} from 100 pM to 260 pM. In embodiments, the IgG binds EGFR with a K_{D} from 100 pM to 240 pM. In embodiments, the IgG binds EGFR with a K_{D} from 100 pM to 220 pM. In embodiments, the IgG binds EGFR with a K_{D} from 100 pM to 200 pM. In embodiments, the IgG binds EGFR with a K_{D} from 100 pM to 180 pM. In embodiments, the IgG binds EGFR with a K_{D} from 100 pM to 160 pM. In embodiments, the IgG binds EGFR with a K_{D} from 100 pM to 140 pM. In embodiments, the IgG binds EGFR with a K_{D} from 100 pM to 120 pM.

In embodiments, the IgG binds EGFR with a K_{D} of about 100 pM, 120 pM, 140 pM, 160 pM, 180 pM, 200 pM, 220 pM, 240 pM, 260 pM, 280 pM, 300 pM, 320 pM, 340 pM, 360 pM, 400 pM, 420 pM, 440 pM, 460 pM, 480 pM, 500 pM, 520 pM, 540 pM, 560 pM, 580 pM, 600 pM, 620 pM, 640 pM, 660 pM, 680 pM, 700 pM, 720 pM, 740 pM, 760 pM, 780 pM, 800 pM, 820 pM, 840 pM, 860 pM, 880 pM, 900 pM, 920 pM, 940 pM, 960 pM, 980 pM, or 1000 pM. In embodiments, the IgG binds EGFR with a K_{D} of 100 pM, 120 pM, 140 pM, 160 pM, 180 pM, 200 pM, 220 pM, 240 pM, 260 pM, 280 pM, 300 pM, 320 pM, 340 pM, 360 pM, 400 pM, 420 pM, 440 pM, 460 pM, 480 pM, 500 pM, 520 pM, 540 pM, 560 pM, 580 pM, 600 pM, 620 pM, 640 pM, 660 pM, 680 pM, 700 pM, 720 pM, 740 pM, 760 pM, 780 pM, 800 pM, 820 pM, 840 pM, 860 pM, 880 pM, 900 pM, 920 pM, 940 pM, 960 pM, 980 pM, or 1000 pM.

In embodiments, the IgG binds EGFR with a K_{D} of about 487 pM. In embodiments, the IgG binds EGFR with a K_{D} of 487 pM. In embodiments, the IgG binds EGFR with a K_{D} of about 214 pM. In embodiments, the IgG binds EGFR with a K_{D} of 214 pM.

The antibodies provided herein including embodiments thereof are capable of binding to EGFR. In embodiments, the antibody binds to EGFR. In embodiments, the antibody binds to domain IV of EGFR. In embodiments, domain IV of EGFR includes the amino acid sequence of SEQ ID NO:276. In embodiments, domain IV of EGFR is the amino acid sequence of SEQ ID NO:276. In embodiments, the antibody binds to the amino acid sequence of SEQ ID NO:276. In embodiments, the antibody binds to an amino acid sequence with 75%, 80%, 85%, 90, 95%, 98% or 99% sequence identity to human domain IV EGFR, variants or homologs thereof. In embodiments, the antibody binds to an amino acid sequence with 75%, 80%, 85%, 90, 95%, 98% or 99% sequence identity to the amino acid sequence of SEQ ID NO:276. In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) of domain IV EGFR. In embodiments, the antibody binds to an amino acid sequence with at least 75%, 80%, 85%, 90, 95%, 98% or 99% sequence identity to human domain IV EGFR, wherein the identity is across the whole sequence or a portion of the sequence (e.g. a 50, 100, 150 or 200 continuous amino acid portion). In embodiments, the antibody binds to an amino acid sequence with at least 75%, 80%, 85%, 90, 95%, 98% or 99% sequence identity to SEQ ID NO:276, wherein the identity is across the whole sequence or a portion of the sequence (e.g. a 50, 100, 150 or 200 continuous amino acid portion).

In embodiments, the domain IV EGFR is substantially identical to the domain IV of the protein identified by the UniProt reference number P00533 or a variant or homolog having substantial identity thereto. In embodiments, the domain IV EGFR has at least 90%, 95%, 96%, 97%, 98%, 99% or 100% amino acid sequence identity across the whole sequence or a portion of the sequence (e.g. a 50, 100, 150 or 200 continuous amino acid portion) of the sequence of SEQ ID NO:276.

In embodiments, the EGFR includes the amino acid sequence of SEQ ID NO:273, SEQ ID NO:274 or SEQ ID NO:275. In embodiments, the EGFR includes the amino acid sequence of SEQ ID NO:273. In embodiments, the EGFR includes the amino acid sequence of SEQ ID NO:274. In embodiments, the EGFR includes the amino acid sequence of SEQ ID NO:275.

In embodiments, the antibody binds the amino acid sequence of SEQ ID NO:273, SEQ ID NO:274 or SEQ ID NO:275. In embodiments, the antibody binds the amino acid sequence of SEQ ID NO:273. In embodiments, the antibody binds the amino acid sequence of SEQ ID NO:274. In embodiments, the antibody binds the amino acid sequence of SEQ ID NO:275. Thus, in an aspect is provided an antibody provided herein including embodiments thereof bound to domain IV of EGFR.

Further provided herein are antibodies capable of binding the same EGFR epitope (e.g. domain IV of EGFR) that is bound by the antibodies provided herein including embodiments thereof. Thus, in an aspect is provided an anti-EGFR antibody, wherein the anti-EGFR antibody binds the same epitope as an antibody including: a heavy chain variable domain including any one of the combinations of a CDR 1 sequence, a CDR2 sequence and a CDR3 sequence set forth by Table 1, 3, 5 or 7; and a light chain variable domain comprising any one of the combinations of a CDR 1 sequence, a CDR2 sequence and a CDR3 sequence set forth by Table 2, 4, 6 or 8. For example, the antibody may bind the same epitope as an anti-EGFR antibody including a heavy chain variable domain including: a CDR H1 as set forth in SEQ ID NO:199, a CDR H2 as set forth in SEQ ID NO:200 and a CDR H3 as set forth in SEQ ID NO:201; and a light chain variable domain including: a CDR L1 as set forth in SEQ ID NO:202, a CDR L2 as set forth in SEQ ID NO:203, and a CDR L3 as set forth in SEQ ID NO:204.

In an aspect is provided an anti-EGFR antibody, wherein the anti-EGFR antibody binds the same epitope as an antibody including: a heavy chain variable domain including a CDR H1 as set forth in SEQ ID NO:199, a CDR H2 as set forth in SEQ ID NO:200 and a CDR H3 as set forth in SEQ ID NO:201; and a light chain variable domain including a CDR L1 as set forth in SEQ ID NO:202, a CDR L2 as set forth in SEQ ID NO:203, and a CDR L3 as set forth in SEQ ID NO:204. In embodiments, the heavy chain sequence has the sequence of SEQ ID NO:271 and the light chain sequence has the sequence of SEQ ID NO:272.

In an aspect is provided an anti-EGFR antibody, wherein the anti-EGFR antibody binds the same epitope as an antibody including: a heavy chain variable domain including a CDR H1 as set forth in SEQ ID NO:103, a CDR H2 as set forth in SEQ ID NO:104 and a CDR H3 as set forth in SEQ ID NO:105; and a light chain variable domain including a CDR L1 as set forth in SEQ ID NO:136, a CDR L2 as set forth in SEQ ID NO:137, and a CDR L3 as set forth in SEQ ID NO:138. In embodiments, the heavy chain sequence has the sequence of SEQ ID NO:249 and said light chain sequence has the sequence of SEQ ID NO:250

In an aspect is provided an anti-EGFR antibody, wherein the anti-EGFR antibody binds the same epitope as an antibody including: a heavy chain variable domain including a CDR H1 as set forth in SEQ ID NO:148, a CDR H2 as set forth in SEQ ID NO:149 and a CDR H3 as set forth in SEQ ID NO:150; and a light chain variable domain including a CDR L1 as set forth in SEQ ID NO:178, a CDR L2 as set forth in SEQ ID NO:179, and a CDR L3 as set forth in SEQ ID NO:180. In embodiments, the heavy chain sequence has the sequence of SEQ ID NO:257 and said light chain sequence has the sequence of SEQ ID NO:258.

In an aspect is provided an anti-EGFR antibody, wherein the anti-EGFR antibody binds the same epitope as an antibody including: a heavy chain variable domain including a CDR H1 as set forth in SEQ ID NO:100, a CDR H2 as set forth in SEQ ID NO:101 and a CDR H3 as set forth in SEQ ID NO:102; and a light chain variable domain including a CDR L1 as set forth in SEQ ID NO:133, a CDR L2 as set forth in SEQ ID NO:134, and a CDR L3 as set forth in SEQ ID NO:135. In embodiments, the heavy chain sequence has the sequence of SEQ ID NO:247 and said light chain sequence has the sequence of SEQ ID NO:248.

In embodiments, the antibody includes any one of the heavy chain sequences set forth by Table 9, 10, 11 or 12. In embodiments, the antibody includes any one of the heavy chain sequences set forth by Table 9. In embodiments, the antibody includes any one of the heavy chain sequences set forth by Table 10. In embodiments, the antibody includes any one of the heavy chain sequences set forth by Table 11. In embodiments, the antibody includes any one of the heavy chain sequences set forth by Table 12. In embodiments, the antibody includes any one of the light chain sequences set forth by Table 9, 10, 11 or 12. In embodiments, the antibody includes any one of the light chain sequences set forth by Table 9. In embodiments, the antibody includes any one of the light chain sequences set forth by Table 10. In embodiments, the antibody includes any one of the light chain sequences set forth by Table 11. In embodiments, the antibody includes any one of the light chain sequences set forth by Table 12. In embodiments, the antibody includes one of the heavy chain sequence and light chain sequence combinations set forth by Table 9, 10, 11 or 12. In embodiments, the antibody includes one of the heavy chain sequence and light chain sequence combinations set forth by Table 9. In embodiments, the antibody includes one of the heavy chain sequence and light chain sequence combinations set forth by Table 10. In embodiments, the antibody includes one of the heavy chain sequence and light chain sequence combinations set forth by Table 11. In embodiments, the antibody includes one of the heavy chain sequence and light chain sequence combinations set forth by Table 12.

In embodiments, the anti-EGFR antibody is capable of binding to EGFR. In embodiments, the anti-EGFR antibody is capable of binding domain IV of EGFR. In embodiments, the anti-EGFR antibody does not substantially bind to domain I, domain II or domain III of EGFR.

Thus, in an aspect is provided an antibody provided herein including embodiments thereof bound to domain IV of EGFR.

### Nucleic Acid Compositions

A gene encoding a modified endogenous cell surface molecule (e.g., tEGFR) may be used as a cell selection or enrichment marker for a genetically modified population of immune cells (e.g., T cells). The gene encoding a modified endogenous cell surface molecule (e.g., tEGFR) may be coupled to a gene encoding a tumor targeting chimeric antigen receptor (CAR). These genes may be inserted into a vector to transduce the population of T cells to be genetically modified. After transduction, the cells that are successfully transduced and express the CAR and modified endogenous cell-surface molecule (e.g., tEGFR) are enriched by any suitable purification method, such as immunomagnetic purification with anti-biotin microbeads or fluorochrome-conjugated anti-biotin for fluorescence activated cell sorting, using a commercial antibody that recognizes the modified endogenous cell-surface molecule expressed by the transduced cell.

In another embodiment, a gene encoding a truncated human epidermal growth factor receptor (EGFRt) that lacks the membrane distal EGF-binding domain and the cytoplasmic signaling tail, but retains domain IV EGFR recognized by any of the antibodies provided herein including embodiments thereof. The EGFRt may be coupled with chimeric antigen receptors specific for a tumor associated antigen. The tumor associated antigen may be CD19, CD20, or CD22, or any other tumor associated antigen, but is preferably CD19 (CD19CAR). The tumor associated antigen is followed by a C-terminal 2A cleavable linker and the coding sequence for EGFRt. The biotinylated-antibody may be used in conjunction with commercially available anti-biotin microbeads for the purpose of immunomagnetic purification of the tumor associated antigen/CAR-expressing transductants. In the instance where the tumor associated antigen is CD19 the product is CD19CAR-expressing transductants. Alternatively, the biotinylated-antibody may be used in conjunction with Fluorochrome-conjugated anti-biotin for fluorescence activated cell sorting.

In an aspect is provided a recombinant nucleic acid including a sequence encoding a truncated EGFR (tEGFR) cell surface molecule, wherein the tEGFR cell surface molecule includes an EGFR domain IV and does not include an EGFR domain III. In embodiments, the tEGFR cell surface molecule does not include an EGFR domain I, an EGFR domain II, an EGFR juxtamembrane domain or an EGFR tyrosine kinase domain. In embodiments, the tEGFR cell surface molecule is non-immunogenic.

In embodiments, the tEGFR cell surface molecule is a human tEGFR cell surface molecule. In embodiments, the tEGFR surface molecule includes a tEGFR transmembrane domain. The tEGFR transmembrane domain anchors the tEGFR surface molecule in the cellular membrane of the cell expressing the tEGFR surface molecule. The tEGFR surface molecule may include one or more cytoplasmic amino acids. A cytoplasmic amino acid as provided herein refers to an amino acid that is attached to a tEGFR transmembrane domain and that is located on the intracellular side of the cellular membrane the transmembrane domain is spanning. In embodiments, the tEGFR surface molecule does not include more than five cytoplasmic amino acids. In embodiments, the tEGFR surface molecule does not include more than four cytoplasmic amino acids. In embodiments, the tEGFR surface molecule does not include more than three cytoplasmic amino acids. In embodiments, the tEGFR surface molecule does not include more than two cytoplasmic amino acids. In embodiments, the tEGFR surface molecule does not include a cytoplasmic amino acid.

In embodiments, the tEGFR cell surface molecule includes an amino acid sequence having a sequence identity of at least 85% to the amino acid sequence of SEQ ID NO:276. In embodiments, the tEGFR cell surface molecule includes an amino acid sequence having a sequence identity of at least 86% to the amino acid sequence of SEQ ID NO:276. In embodiments, the tEGFR cell surface molecule includes an amino acid sequence having a sequence identity of at least 87% to the amino acid sequence of SEQ ID NO:276. In embodiments, the tEGFR cell surface molecule includes an amino acid sequence having a sequence identity of at least 88% to the amino acid sequence of SEQ ID NO:276. In embodiments, the tEGFR cell surface molecule includes an amino acid sequence having a sequence identity of at least 89% to the amino acid sequence of SEQ ID NO:276. In embodiments, the tEGFR cell surface molecule includes an amino acid sequence having a sequence identity of at least 90% to the amino acid sequence of SEQ ID NO:276. In embodiments, the tEGFR cell surface molecule includes an amino acid sequence having a sequence identity of at least 91% to the amino acid sequence of SEQ ID NO:276. In embodiments, the tEGFR cell surface molecule includes an amino acid sequence having a sequence identity of at least 92% to the amino acid sequence of SEQ ID NO:276. In embodiments, the tEGFR cell surface molecule includes an amino acid sequence having a sequence identity of at least 93% to the amino acid sequence of SEQ ID NO:276. In embodiments, the tEGFR cell surface molecule includes an amino acid sequence having a sequence identity of at least 94% to the amino acid sequence of SEQ ID NO:276. In embodiments, the tEGFR cell surface molecule includes an amino acid sequence having a sequence identity of at least 95% to the amino acid sequence of SEQ ID NO:276. In embodiments, the tEGFR cell surface molecule includes an amino acid sequence having a sequence identity of at least 96% to the amino acid sequence of SEQ ID NO:276. In embodiments, the tEGFR cell surface molecule includes an amino acid sequence having a sequence identity of at least 97% to the amino acid sequence of SEQ ID NO:276. In embodiments, the tEGFR cell surface molecule includes an amino acid sequence having a sequence identity of at least 98% to the amino acid sequence of SEQ ID NO:276. In embodiments, the tEGFR cell surface molecule includes an amino acid sequence having a sequence identity of at least 99% to the amino acid sequence of SEQ ID NO:276. In embodiments, the tEGFR cell surface molecule includes an amino acid sequence having a sequence identity of at least 85%, 86%, 87%, 88%, 89%, 90%, 91%, 92%, 93%, 94%, 95%, 96%, 97%, 98% or 99% to the amino acid sequence of SEQ ID NO:276. In further embodiments, the tEGFR cell surface molecule includes a tEGFR transmembrane domain including the amino acid sequence of SEQ ID NO:283. In further embodiments, the tEGFR cell surface molecule includes a tEGFR transmembrane domain with the amino acid sequence of SEQ ID NO:283. In embodiments, the tEGFR cell surface molecule is the amino acid sequence of SEQ ID NO:276.

In embodiments, the tEGFR cell surface molecule includes an amino acid sequence having a sequence identity of at least 85% to the amino acid sequence of SEQ ID NO:285. In embodiments, the tEGFR cell surface molecule includes an amino acid sequence having a sequence identity of at least 86% to the amino acid sequence of SEQ ID NO:285. In embodiments, the tEGFR cell surface molecule includes an amino acid sequence having a sequence identity of at least 87% to the amino acid sequence of SEQ ID NO:285. In embodiments, the tEGFR cell surface molecule includes an amino acid sequence having a sequence identity of at least 88% to the amino acid sequence of SEQ ID NO:285. In embodiments, the tEGFR cell surface molecule includes an amino acid sequence having a sequence identity of at least 89% to the amino acid sequence of SEQ ID NO:285. In embodiments, the tEGFR cell surface molecule includes an amino acid sequence having a sequence identity of at least 90% to the amino acid sequence of SEQ ID NO:285. In embodiments, the tEGFR cell surface molecule includes an amino acid sequence having a sequence identity of at least 91% to the amino acid sequence of SEQ ID NO:285. In embodiments, the tEGFR cell surface molecule includes an amino acid sequence having a sequence identity of at least 92% to the amino acid sequence of SEQ ID NO:285. In embodiments, the tEGFR cell surface molecule includes an amino acid sequence having a sequence identity of at least 93% to the amino acid sequence of SEQ ID NO:285. In embodiments, the tEGFR cell surface molecule includes an amino acid sequence having a sequence identity of at least 94% to the amino acid sequence of SEQ ID NO:285. In embodiments, the tEGFR cell surface molecule includes an amino acid sequence having a sequence identity of at least 95% to the amino acid sequence of SEQ ID NO:285. In embodiments, the tEGFR cell surface molecule includes an amino acid sequence having a sequence identity of at least 96% to the amino acid sequence of SEQ ID NO:285. In embodiments, the tEGFR cell surface molecule includes an amino acid sequence having a sequence identity of at least 97% to the amino acid sequence of SEQ ID NO:285. In embodiments, the tEGFR cell surface molecule includes an amino acid sequence having a sequence identity of at least 98% to the amino acid sequence of SEQ ID NO:285. In embodiments, the tEGFR cell surface molecule includes an amino acid sequence having a sequence identity of at least 99% to the amino acid sequence of SEQ ID NO:285. In embodiments, the tEGFR cell surface molecule includes an amino acid sequence having a sequence identity of at least 85%, 86%, 87%, 88%, 89%, 90%, 91%, 92%, 93%, 94%, 95%, 96%, 97%, 98% or 99% to the amino acid sequence of SEQ ID NO:285. In embodiments, the tEGFR cell surface molecule is the amino acid sequence of SEQ ID NO:285.

In embodiments, the tEGFR cell surface molecule includes an amino acid sequence of SEQ ID NO:276 and an amino acid sequence of SEQ ID NO:283. In embodiments, the tEGFR cell surface molecule includes a tEGFR domain IV domain including the amino acid sequence of SEQ ID NO:276 and a tEGFR transmembrane domain including the amino acid sequence of SEQ ID NO:283. In embodiments, the tEGFR cell surface molecule includes a tEGFR domain IV domain with the amino acid sequence of SEQ ID NO:276 and a tEGFR transmembrane domain with the amino acid sequence of SEQ ID NO:283. In further embodiments the tEGFR cell surface molecule further includes a tEGFR signal peptide including the amino acid sequence of SEQ ID NO:284. In further embodiments, the tEGFR cell surface molecule further includes a tEGFR signal peptide with the amino acid sequence of SEQ ID NO:284.

In embodiments, the tEGFR transmembrane domain includes an amino acid sequence having a sequence identity of at least 85% to the amino acid sequence of SEQ ID NO:283. In embodiments, the tEGFR transmembrane domain includes an amino acid sequence having a sequence identity of at least 86% to the amino acid sequence of SEQ ID NO:283. In embodiments, the tEGFR transmembrane domain includes an amino acid sequence having a sequence identity of at least 87% to the amino acid sequence of SEQ ID NO:283. In embodiments, the tEGFR transmembrane domain includes an amino acid sequence having a sequence identity of at least 88% to the amino acid sequence of SEQ ID NO:283. In embodiments, the tEGFR transmembrane domain includes an amino acid sequence having a sequence identity of at least 89% to the amino acid sequence of SEQ ID NO:283. In embodiments, the tEGFR transmembrane domain includes an amino acid sequence having a sequence identity of at least 90% to the amino acid sequence of SEQ ID NO:283. In embodiments, the tEGFR transmembrane domain includes an amino acid sequence having a sequence identity of at least 91% to the amino acid sequence of SEQ ID NO:283. In embodiments, the tEGFR transmembrane domain includes an amino acid sequence having a sequence identity of at least 92% to the amino acid sequence of SEQ ID NO:283. In embodiments, the tEGFR transmembrane domain includes an amino acid sequence having a sequence identity of at least 93% to the amino acid sequence of SEQ ID NO:283. In embodiments, the tEGFR transmembrane domain includes an amino acid sequence having a sequence identity of at least 94% to the amino acid sequence of SEQ ID NO:283. In embodiments, the tEGFR transmembrane domain includes an amino acid sequence having a sequence identity of at least 95% to the amino acid sequence of SEQ ID NO:283. In embodiments, the tEGFR transmembrane domain includes an amino acid sequence having a sequence identity of at least 96% to the amino acid sequence of SEQ ID NO:283. In embodiments, the tEGFR transmembrane domain includes an amino acid sequence having a sequence identity of at least 97% to the amino acid sequence of SEQ ID NO:283. In embodiments, the tEGFR transmembrane domain includes an amino acid sequence having a sequence identity of at least 98% to the amino acid sequence of SEQ ID NO:283. In embodiments, the tEGFR transmembrane domain includes an amino acid sequence having a sequence identity of at least 99% to the amino acid sequence of SEQ ID NO:283. In embodiments, the tEGFR transmembrane domain includes an amino acid sequence having a sequence identity of at least 85%, 86%, 87%, 88%, 89%, 90%, 91%, 92%, 93%, 94%, 95%, 96%, 97%, 98% or 99% to the amino acid sequence of SEQ ID NO:283. In embodiments, the tEGFR transmembrane domain is the amino acid sequence of SEQ ID NO:283.

In embodiments, the tEGFR cell surface molecule has a sequence identity of at least 85% to the amino acid sequence of SEQ ID NO:276. In embodiments, the tEGFR cell surface molecule has a sequence identity of at least 86% to the amino acid sequence of SEQ ID NO:276. In embodiments, the tEGFR cell surface molecule has a sequence identity of at least 87% to the amino acid sequence of SEQ ID NO:276. In embodiments, the tEGFR cell surface molecule has a sequence identity of at least 88% to the amino acid sequence of SEQ ID NO:276. In embodiments, the tEGFR cell surface molecule has a sequence identity of at least 89% to the amino acid sequence of SEQ ID NO:276. In embodiments, the tEGFR cell surface molecule has a sequence identity of at least 90% to the amino acid sequence of SEQ ID NO:276. In embodiments, the tEGFR cell surface molecule has a sequence identity of at least 91% to the amino acid sequence of SEQ ID NO:276. In embodiments, the tEGFR cell surface molecule has a sequence identity of at least 92% to the amino acid sequence of SEQ ID NO:276. In embodiments, the tEGFR cell surface molecule has a sequence identity of at least 93% to the amino acid sequence of SEQ ID NO:276. In embodiments, the tEGFR cell surface molecule has a sequence identity of at least 94% to the amino acid sequence of SEQ ID NO:276. In embodiments, the tEGFR cell surface molecule has a sequence identity of at least 95% to the amino acid sequence of SEQ ID NO:276. In embodiments, the tEGFR cell surface molecule has a sequence identity of at least 96% to the amino acid sequence of SEQ ID NO:276. In embodiments, the tEGFR cell surface molecule has a sequence identity of at least 97% to the amino acid sequence of SEQ ID NO:276. In embodiments, the tEGFR cell surface molecule has a sequence identity of at least 98% to the amino acid sequence of SEQ ID NO:276. In embodiments, the tEGFR cell surface molecule has a sequence identity of at least 99% to the amino acid sequence of SEQ ID NO:276. In embodiments, tEGFR cell surface molecule has a sequence identity of at least 85%, 86%, 87%, 88%, 89%, 90%, 91%, 92%, 93%, 94%, 95%, 96%, 97%, 98% or 99% to the amino acid sequence of SEQ ID NO:276.

For the recombinant nucleic acid provided herein, in embodiments, the tEGFR cell surface molecule binds an anti-domain IV EGFR antibody. In embodiments, the tEGFR cell surface molecule does not bind an anti-domain III EGFR antibody.

In embodiments, the recombinant nucleic acid further includes a sequence encoding a chimeric antigen receptor, a T cell receptor, or a cytokine receptor. In embodiments, the recombinant nucleic acid further includes a sequence encoding a chimeric antigen receptor. In embodiments, the recombinant nucleic acid further includes a sequence encoding a T cell receptor. In embodiments, the recombinant nucleic acid further includes a sequence encoding a cytokine receptor.

In embodiments, the chimeric antigen receptor includes an antibody region and a transmembrane domain. In embodiments, the antibody region binds to a cancer-antigen. In embodiments, the antibody region binds to CD19. In embodiments, the sequence encoding the chimeric antigen receptor further includes an intracellular T-cell signaling domain. In embodiments, the cytokine receptor is IL15. In embodiments, the recombinant nucleic acid further includes a sequence encoding a self-cleaving peptidyl sequence. In embodiments, the self-cleaving peptidyl sequence connects the sequence encoding the tEGFR cell surface molecule with the sequence encoding the chimeric antigen receptor.

For the recombinant nucleic acid provided herein, in embodiments, the self-cleaving peptidyl sequence encodes a T2A peptidyl sequence, P2A peptidyl sequence, a E2A peptidyl sequence, a F2A peptidyl sequence or a 2A peptidyl sequence. In embodiments, the self-cleaving peptidyl sequence encodes a T2A peptidyl sequence. In embodiments, the self-cleaving peptidyl sequence encodes a P2A peptidyl sequence. In embodiments, the self-cleaving peptidyl sequence encodes a E2A peptidyl sequence. In embodiments, the self-cleaving peptidyl sequence encodes a F2A peptidyl sequence. In embodiments, the self-cleaving peptidyl sequence encodes a 2A peptidyl sequence.

In an aspect is provided an expression vector including the recombinant nucleic acid provided herein including embodiments thereof. In embodiments, the expression vector is an adenoviral vector or a retroviral vector. In embodiments, the expression vector is an adenoviral vector. In embodiments, the expression vector is a retroviral vector.

### Cells

In an aspect is provided a cell including a tEGFR cell surface molecule provided herein including embodiments thereof or an expression vector provided herein including embodiments thereof. In embodiments, the cell is bound to an anti-domain IV EGFR antibody in vitro or in vivo. In embodiments, the cell is bound to an anti-domain IV EGFR antibody in vitro. In embodiments, the cell is bound to an anti-domain IV EGFR antibody in vivo.

In embodiments, the cell is a T cell, a natural killer (Nk) cell or an induced pluripotent stem cell (iPSC). In embodiments, the cell is a T cell. In embodiments, the cell is a natural killer (Nk) cell. In embodiments, the cell is an induced pluripotent stem cell (iPSC). In embodiments, the Nk cell is a cytoprotective Nk cell. In embodiments, the Nk cell is a cytotoxic NK cell.

### Kits

In an aspect is provided a kit composition including (i) an expression vector provided herein including embodiments thereof; and (ii) an anti-domain IV EGFR antibody or an expression vector encoding an anti-domain IV EGFR antibody. In embodiments, the expression vector of (i) and the expression vector or said antibody of (ii) are in separate containers.

### Methods of Selecting Cells

A method of selecting a cell expressing a tEGFR cell surface molecule, the method including: (i) contacting a population of cells with a recombinant nucleic acid provided herein including embodiments thereof or an expression vector provided herein including embodiments thereof, thereby forming a contacted cell population: (ii) contacting the contacted cell population with a tEGFR binding agent, thereby forming a bound tEGFR expressing cell; and (iii) separating the bound tEGFR expressing cell from the contacted cell population, thereby selecting a cell expressing a tEGFR cell surface molecule. In embodiments, the tEGFR binding agent is an anti-domain IV EGFR antibody. In embodiments, the antibody includes a detectable moiety. In embodiments, the population of cells is in a subject. In embodiments, the population of cells is in a tissue culture container.

### Methods of Selecting Cells

In an aspect is provided a method of detecting a cell expressing a tEGFR cell surface molecule, the method including: (i) contacting a population of cells expressing a recombinant nucleic acid provided herein including embodiments thereof or an expression vector provided herein including embodiments thereof with a tEGFR binding agent, and (ii) detecting binding of the binding agent to a tEGFR cell surface molecule thereby detecting a cell expressing a tEGFR cell surface molecule. In embodiments, the tEGFR binding agent is an anti-domain IV EGFR antibody. In embodiments, the antibody includes a detectable moiety.

Also provided are methods for identifying new therapeutic cell products having the following criteria: a modified endogenous cell-surface molecule, ligand or receptor that is not, as modified, endogenously expressed in the subject in which it is intended to be therapeutically utilized, does not have any immunoactivity or other functional activity that would hinder the functioning of the product or the subject into which the product is administered, and that it can be recognized by an anti-domain IV antibody (e.g., the anti-domain IV antibody provided herein).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Embodiments

Embodiment 1. A recombinant nucleic acid comprising a sequence encoding a truncated EGFR (tEGFR) cell surface molecule, wherein said tEGFR cell surface molecule comprises an EGFR domain IV and does not comprise an EGFR domain III.

Embodiment 2. The recombinant nucleic acid of embodiment 1, wherein said tEGFR cell surface molecule does not comprise an EGFR domain I, an EGFR domain II, an EGFR juxtamembrane domain or an EGFR tyrosine kinase domain.

Embodiment 3. The recombinant nucleic acid of embodiment 1 or 2, wherein said tEGFR cell surface molecule is non-immunogenic.

Embodiment 4. The recombinant nucleic acid of any one of embodiments 1-3, wherein said tEGFR cell surface molecule is a human tEGFR cell surface molecule.

Embodiment 5. The recombinant nucleic acid of any one of claims 1-4, wherein said tEGFR cell surface molecule has a sequence identity of at least 85% to the amino acid sequence of SEQ ID NO:276.

Embodiment 6. The recombinant nucleic acid of any one of claims 1-5, wherein said tEGFR cell surface molecule binds an anti-domain IV EGFR antibody.

Embodiment 7. The recombinant nucleic acid of any one of claims 1-6, wherein said tEGFR cell surface molecule does not bind an anti-domain III EGFR antibody.

Embodiment 8. The recombinant nucleic acid of any one of claims 1-6, further comprising a sequence encoding a chimeric antigen receptor, a T cell receptor, or a cytokine receptor.

Embodiment 9. The recombinant nucleic acid of claim 8, wherein said chimeric antigen receptor comprises an antibody region and a transmembrane domain.

Embodiment 10. The recombinant nucleic acid of claim 9, wherein said antibody region binds to a cancer-antigen.

Embodiment 11. The recombinant nucleic acid of claim 9, wherein said antibody region binds to CD19.

Embodiment 12. The recombinant nucleic acid of any one of claims 8-11, wherein said sequence encoding said chimeric antigen receptor further comprises an intracellular T-cell signaling domain.

Embodiment 13. The recombinant nucleic acid of claim 8, wherein said cytokine receptor is IL15.

Embodiment 14. The recombinant nucleic acid of any one of claims 8-12, further comprising a sequence encoding a self-cleaving peptidyl sequence.

Embodiment 15. The recombinant nucleic acid of claim 14, wherein said self-cleaving peptidyl sequence connects said sequence encoding said tEGFR cell surface molecule with said sequence encoding said chimeric antigen receptor.

Embodiment 16. The recombinant nucleic acid of claim 14 or 15, wherein said self-cleaving peptidyl sequence encodes a T2A peptidyl sequence, P2A peptidyl sequence, a E2A peptidyl sequence, a F2A peptidyl sequence or a 2A peptidyl sequence.

Embodiment 17. An expression vector comprising the recombinant nucleic acid of one of claims 1-16.

Embodiment 18. The expression vector of claim 17, wherein said expression vector is an adenoviral vector or a retroviral vector.

Embodiment 19. The expression vector of claim 18, wherein said retroviral vector is a retroviral vector.

Embodiment 20. A cell comprising a tEGFR cell surface molecule of any one of claims 1-16 or an expression vector of any one of claims 17-18.

Embodiment 21. The cell of claim 20, wherein said cell is bound to an anti-domain IV EGFR antibody in vitro or in vivo.

Embodiment 22. The cell of claim 20 or 21, wherein said cell is a T cell, a natural killer (Nk) cell or an induce pluripotent stem cell (iPSC).

Embodiment 23. A kit composition comprising (i) an expression vector of any one of claims 17-18; and (ii) an anti-domain IV EGFR antibody or an expression vector encoding an anti-domain IV EGFR antibody.

Embodiment 24. The kit of claim 23, wherein said expression vector of (i) and said expression vector or said antibody of (ii) are in separate containers.

Embodiment 25. A method of selecting a cell expressing a tEGFR cell surface molecule, said method comprising: (i) contacting a population of cells with a recombinant nucleic acid of any one of claims 1-16 or an expression vector of any one of claims 17-19, thereby forming a contacted cell population: (ii) contacting said contacted cell population with a tEGFR binding agent, thereby forming a bound tEGFR expressing cell; and (iii) separating said bound tEGFR expressing cell from said contacted cell population, thereby selecting a cell expressing a tEGFR cell surface molecule.

Embodiment 26. The method of claim 25, wherein said tEGFR binding agent is an anti-domain IV EGFR antibody.

Embodiment 27. The method of claim 26, wherein said antibody comprises a detectable moiety.

Embodiment 28. The method of any one of claims 25-27, wherein said population of cells is in a subject.

Embodiment 29. The method of any one of claims 25-27, wherein said population of cells is in a tissue culture container.

Embodiment 30. A method of detecting a cell expressing a tEGFR cell surface molecule, said method comprising: (i) contacting a population of cells expressing a recombinant nucleic acid of any one of claims 1-15 or an expression vector of any one of claims 17-19 with a tEGFR binding agent, and (ii) detecting binding of said binding agent to a tEGFR cell surface molecule thereby detecting a cell expressing a tEGFR cell surface molecule.

Embodiment 31. The method of claim 30, wherein said tEGFR binding agent is an anti-domain IV EGFR antibody.

Embodiment 32. The method of claim 31, wherein said antibody comprises a detectable moiety.

### EXAMPLES

### Example 1: Development of EGFR Domain IV Targeting Antibodies

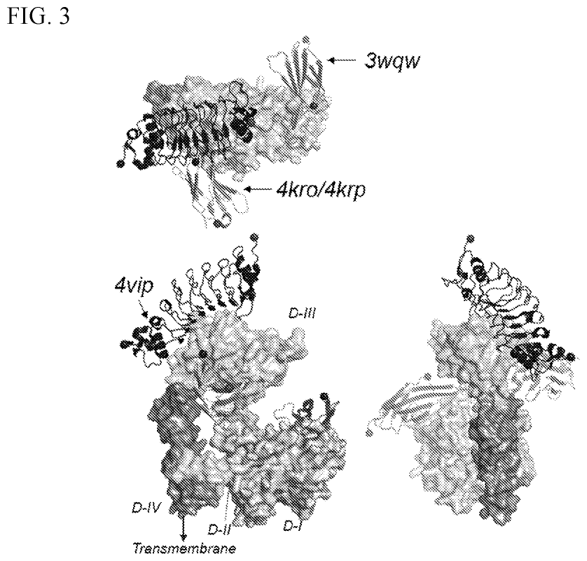

Various EGFR-targeting biologics were investigated for their binding location on the EGFR target. Based on crystallographic data, the superposition of biologics to the extracellular domain indicate that nearly all biologics bind to domain III of the EGFR target (FIG. 2). This is consistent with the mode of action of the biologics, specifically, of EGF blockade as the ligand binds to domain III and is secured by a large conformational change leading to domain I covering the EGF ligand. Likewise, non-antibody EGFR-targeting therapeutics bind to domain III, operating similarly by inhibiting EGF binding (FIG. 3).

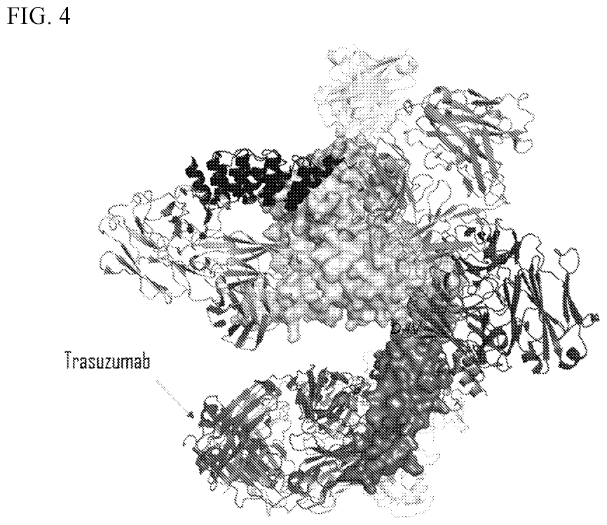

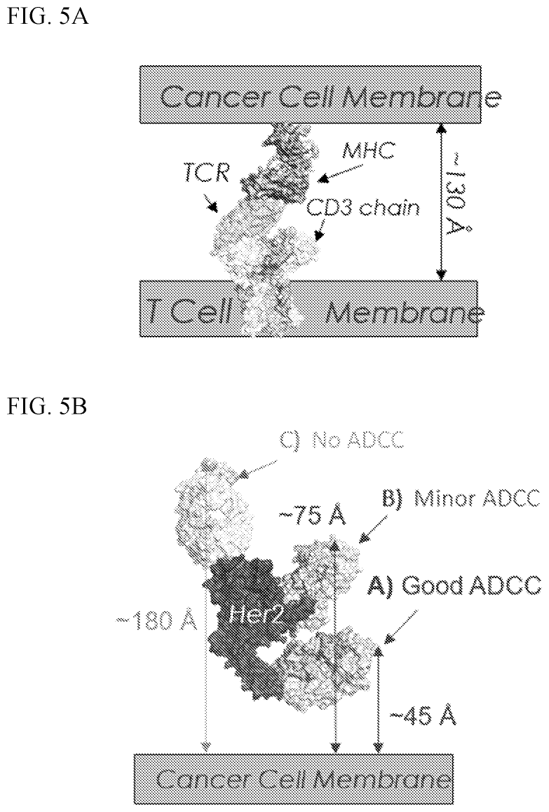

In contrast, trastuzumab, which is a highly effective therapeutic targeting Her2 positive tumors, does not have an extracellular ligand. Trastuzumab binds domain IV of Her2, the juxtamembrane domain (FIG. 4). Thus, the mechanism of action of trastuzumab is distinct from cetuximab. In studies by Applicants, results show that trastuzumab strongly potentiates Antibody-Dependent Cellular Cytotoxicity (ADCC). This is in contrast to the significantly weaker effect that cetuximab induces. Moreover, in combining crystallographic data with T cell activation studies, there appears to be a strong correlation between T cell activation and the proximity of the epitope and membrane distance (FIGS. 5A-5C).

Importantly, the superposition of EGFR-targeting biologics indicates that there are no biologics at least to Applicants' knowledge that bind to domain IV of EGFR. Based on this observation, Applicants developed monoclonal antibodies (mAbs) specifically to target domain IV potently and found that such antibodies activate ADCC surprisingly well. Applicants note that the Fab domains of the anti-domain IV EGFR antibodies bind with low affinity, but the IgG isotype binds with high affinity. The differential binding affinity of the Fab (monovalent binder) compared to the IgG (bivalent binder) suggests selectivity to overexpressed EGFR.

Using multiple cell lines, Applicants show that in embodiments the antibodies provided herein are specific to overexpressing EGFR cell lines which express low or no Her2 expression. This suggests that the antibodies provided herein will have high specificity and safety. That is, side effects that accompany other less specific antibody therapeutics can be avoided. Critically, Applicants show in animal studies that ADCC competent anti-domain IV EGFR mAb eradicates MDA-MB-468 tumor xenograft, a triple negative breast cancer (TNBC) cell line (e.g., no Her2 expression). In contrast, an ADCC-silent variant of the mAb (e.g., swapping Fcs that do not bind CD16) fails to halt tumor growth (e.g., similar to PBS). Moreover, cetuximab re-engineered with the same ADCC competent Fc inhibits tumor growth (but does not eradicate the tumor). Collectively, these extensive data support the development of anti-domain IV EGFR mAbs for treating triple-negative breast cancer (TNBC). In addition, domain IV targeting mAbs may also be useful to treat colorectal cancer and non-small cell lung cancer (NSCLC), also characterized by low or no Her2 expression.

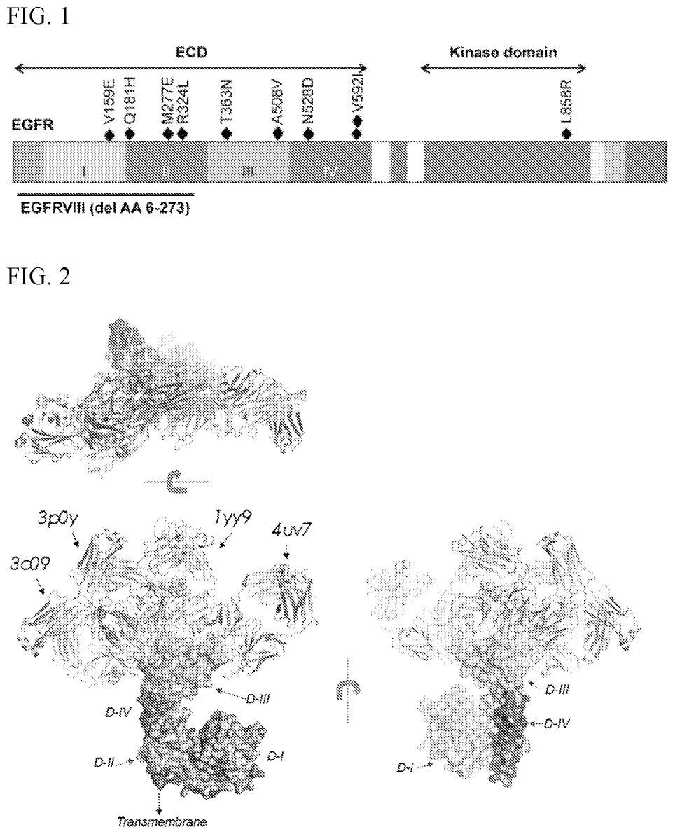

Furthermore, analysis of the co-crystal structure of the Fab domain of anti-domain IV EGFR antibody clone 5C8 with EGFR domain IV is guiding antibody humanization and optimization of the antibody to improve binding affinity to the epitope. The structure provides insight for how the domain IV point mutations (FIG. 1) affect antibody targeting.

### Example 2: Materials and Methods

### Production of Chimeric Human EGFR Domain IV Protein

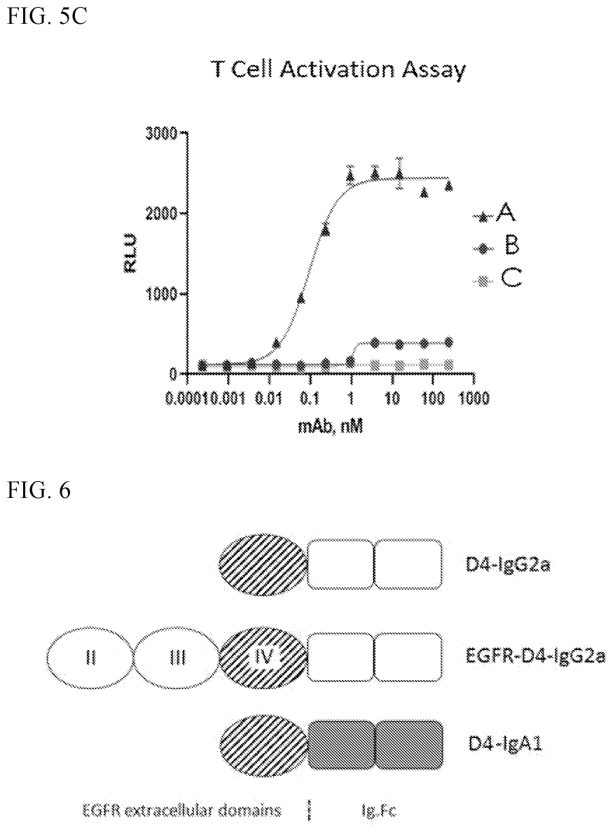

Chimeric human EGFR domain IV proteins were constructed by fusing the extracellular EGFR domain IV, or domains II to IV with mouse IgG2a. Fc, designed as D4-IgG2a (SEQ ID NO:273) and EGFR-D4-IgG2a (SEQ ID NO:274), respectively (FIG. 6). The extracellular domain IV of human EGFR fused with human IgA1.Fc was designated as D4-IgA (SEQ ID NO:275) (FIG. 6). The DNAs encoding these fusion proteins were synthesized and cloned into a mammalian expression vector by the manufacture (Twist Bioscience). The chimeric proteins were produced using an ExpiCHO expression system (Thermo Fisher Scientific). The procedures were followed according to the manufacturer's manual.

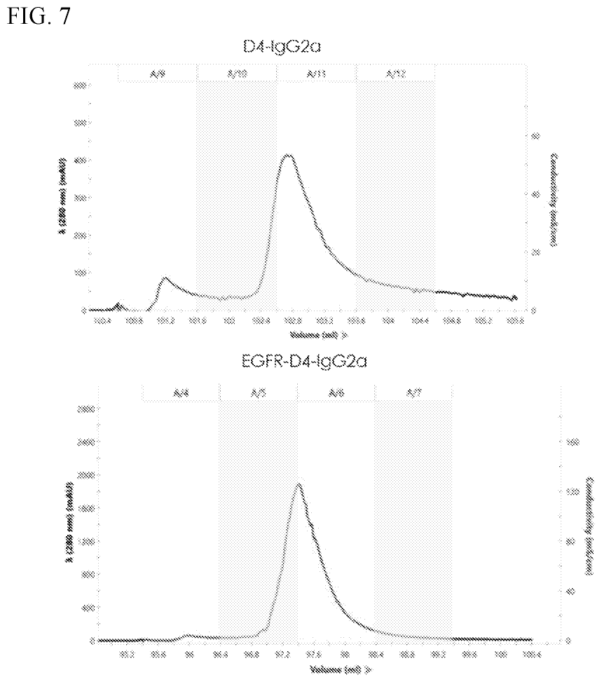

In brief, CHO cells were seeded at 3-4×10⁶ cells/mL in fresh medium one day before transfection. On the next day, cells were adjusted to 6×10⁶ cells/mL. For a 200 mL transfection, 160 μg of DNA was added into 8 mL of OptiPRO™ SFM and then mixed with 640 μL of ExpiFectamine™ diluted in 7.4 mL OptiPRO™ SFM. The mixture was slowly added into the cell culture with gentle swirling, and cells were then cultured at 37° C. for 16-22 h. ExpiCHO™ Enhancer (1.2 mL) and ExpiCHO™ Feed (48 mL) were added into the cell culture, and cells were then cultured at 32° C. for 9-10 days. Cell supernatants were harvested by centrifugation at 4000×g for 30 minutes and passed through 0.22-mm filters for protein purification. Protein A resins (GE Healthcare) and CaptureSelect™ IgA Affinity Matrix (Thermo Fisher Scientific) were used to purify mouse IgG2a and human IgA1 fusion proteins, respectively. Immunogen proteins were further purified with a Superdex 200 Increase 10/300 GL column (GE Healthcare) (FIG. 7).

### Preparations of EGFR Domain IV Hybridomas

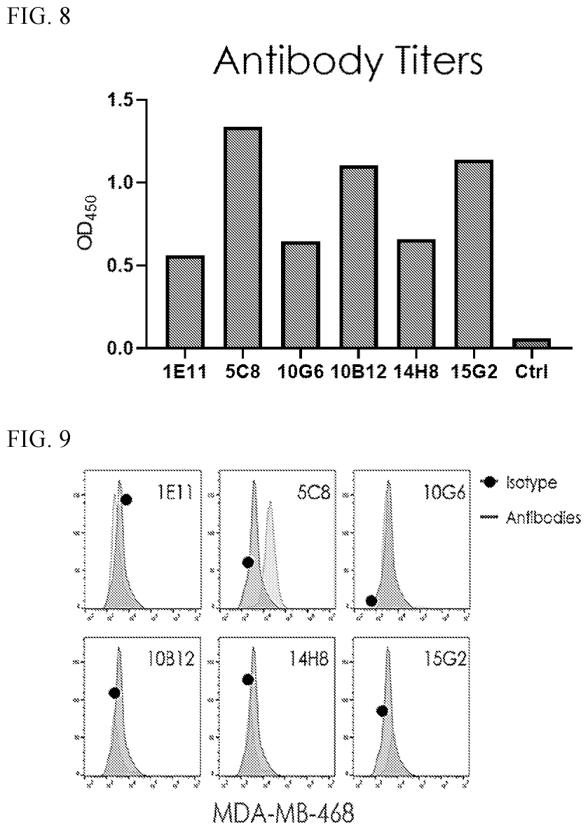

All animal experiments were conducted under the approval of Institutional Animal Care and Use Committee of City of Hope (IUCAC #19070). For mouse immunization, recombinant D4-IgG2a or EGFR-D4 IgG2a fusion proteins were emulsified with complete Freund's adjuvants (Sigma Aldrich) and subcutaneously injected into 10 Balb/c mice (The Jackson Laboratory), respectively. Fifty micrograms of proteins were injected for each mouse. After three weeks, mice received two subcutaneous injections of 50 ug fusion proteins emulsified with incomplete Freund's adjuvants (Sigma Aldrich) in a two-week interval. Three days before spleen harvests, 10 ug of fusion proteins were injected into mice via tail veins. Spleen cells were harvested and fused with mouse myeloma cell line FO (ATCC) at 1:1 ratio using PEG 1500 (Roche). The cell fusion procedures were followed according to the manufacturer's manual. After fusion, cells were selected in complete DMEM medium containing hypoxanthine/aminopterin/thymidine (Thermo Fisher Scientific) and 10% UltraCruz® Hybridoma Cloning Supplement (Santa Cruz) for 10-12 days. Hybridoma culture supernatants were screened for reacting to human EGFR-IgA proteins with ELISAs. To perform ELISA screening, 50 uL of proteins diluted with carbonate/bicarbonate buffer, pH 9.6 at the concentration of 1 ug/mL were added into micro-wells and incubated at 4° C. overnight. Wells were washed with PBS containing 0.1% Tween 20 (PBST) three times and blocked with 200 uL of PBS containing 1% bovine serum albumin (BSA). After incubation at room temperature for 1 h and wash with PBST, 50 uL of culture supernatants were added into wells and incubated at room temperature for 1 hr. After washing, 50 uL of 1:10,000 diluted goat anti-mouse IgG.Fc-HRP (Jackson ImmunoResearch) were added into wells and incubated at room temperature for 1 hr. After washing six times, 50 uL of TMB substrate (Thermo Fisher Scientific) were added into wells for color development. The reactions were stopped by adding 50 μL of 1 N HCl. Wells were read at optical density 450 nm with a Synergy 4 microplate reader (Biotek). Six hybridoma clones were identified from ELISA screening (FIG. 8).

### Flow Cytometry

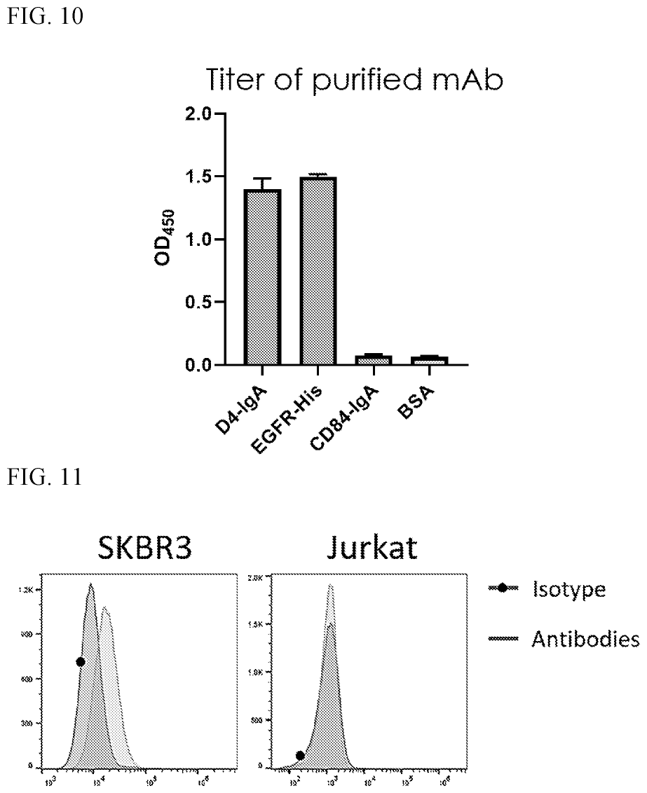

Culture supernatants of hybridoma clones were used to test surface binding for human epithelial cell lines SKOV3 and SKBR3 (ATCC). One million cells were incubated with 100 μL of culture supernatants on ice for 30 min and washed with cold PBS containing 1% FBS and 0.1% sodium azide. Cells were then incubated with 100 μL of 400-fold diluted AffiniPure goat anti-mouse IgG.Fc-AF488 (Jackson ImmunoResearch) on ice for 30 min. Cells were washed with cold PBS containing 1% FBS and 0.1% sodium azide and then analyzed by using an Accuri C6 flow cytometer (BD). Clone 5C8 displayed binding to SKOV3 cells (FIG. 9). The specificity of 5C8 were further tested for reacting to various recombinant proteins and cells lines with ELISA and flow cytometry, respectively (FIGS. 10 and 11).

### Isotyping and Cloning of VH/VL of Anti-EGFR Domain IV Antibody

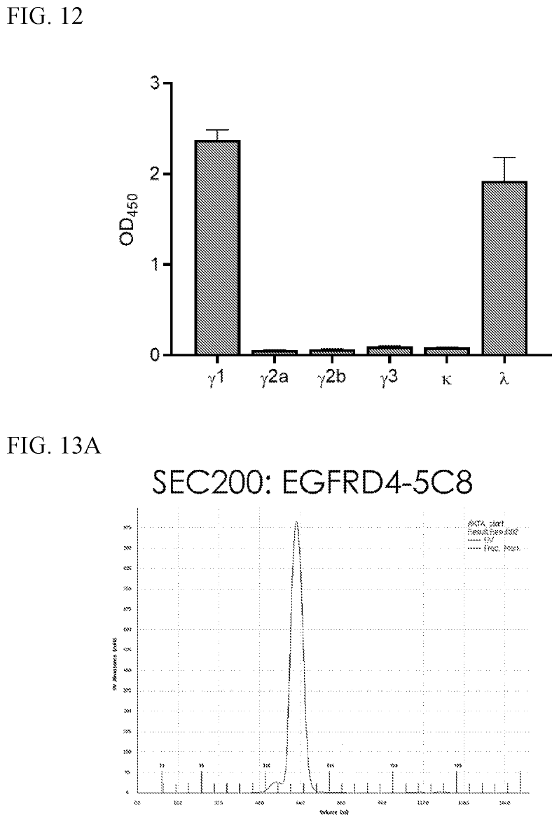

To determine the Ig isotypes of mouse antibodies with ELISAs, D4-IgA proteins were coated onto micro-wells 4° C. overnight. After blocking and washing, hybridoma supernatants were added into wells and incubated for 1 hr. After washing, HRP-conjugated goat anti-mouse Igg1, Igg2a, Igg2b, Igg3, Igk, and Igl (SouthernBiotech) with 1:1,000 dilution were added into wells and incubated for 1 h. Following washing, colors were developed using TMB/HCl and read using a microplate reader. The isotypes of 5C8 were determined to be γ1 and λ. (FIG. 12). To clone VH/VL sequences from hybridomas, mRNAs were extracted using a Quick-RNA Microprep kit (Zymo Research). First-strand cDNAs were synthesized using a SuperScript III First-Strand Synthesis System (Thermo Fisher Scientific). The V_{H} and VL fragments were amplified by PCRs using a Mouse Ig-Primer Set (Millipore Sigma) and OneTaq 2× Master Mix (NEB). Amplified DNA fragments were purified using a DNA Clean-up kit (Zymo Research) and ligated into pGEM-T vectors (Promega) for sequencing.

### Example 3: Generation and Characterization of Anti-Domain IV EGFR

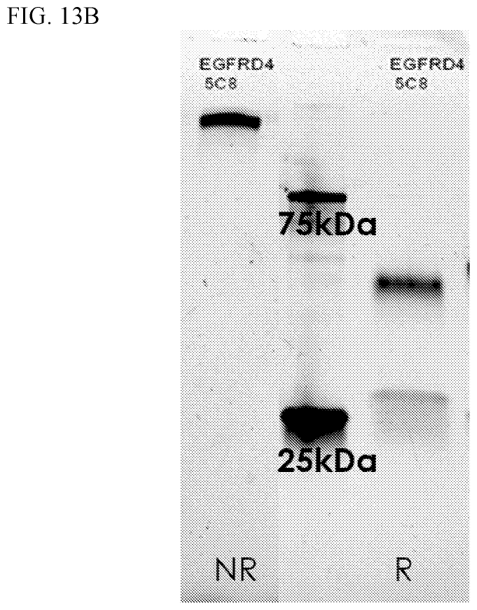

The chimeric anti-domain IV EGFR antibody clone 5C8 (EGFRD4-5C8), which includes murine variable domains (Fvs), was expressed and purified. As a final step, EGFRD4-5C8 was purified by size exclusion chromatography (FIG. 13A), and the purified sample was characterized by both reducing and non-reducing gels. As expected, the non-reducing gel showed a single high molecular weight band, while the reducing gel showed two lower molecular weight bands at approximately 25 kDa and 50 kDa (FIG. 13B).

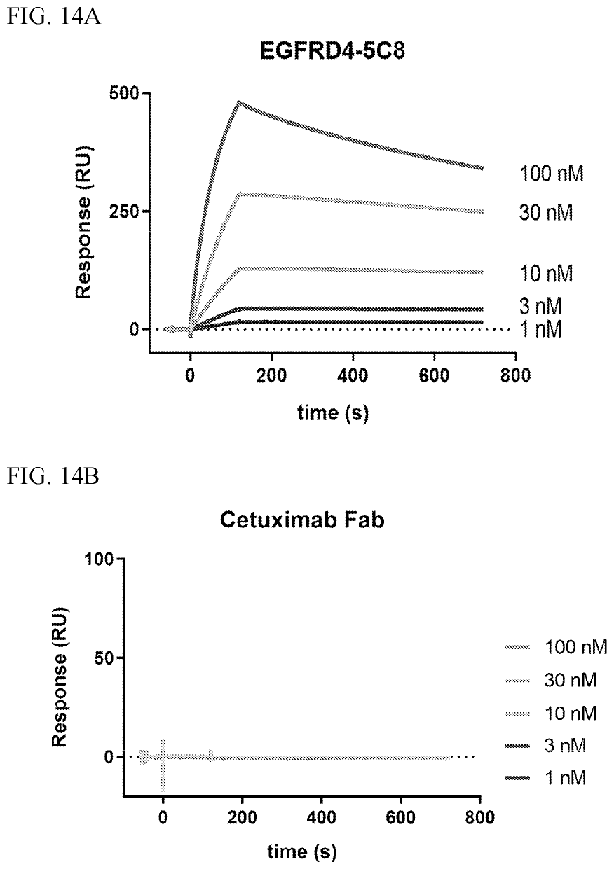

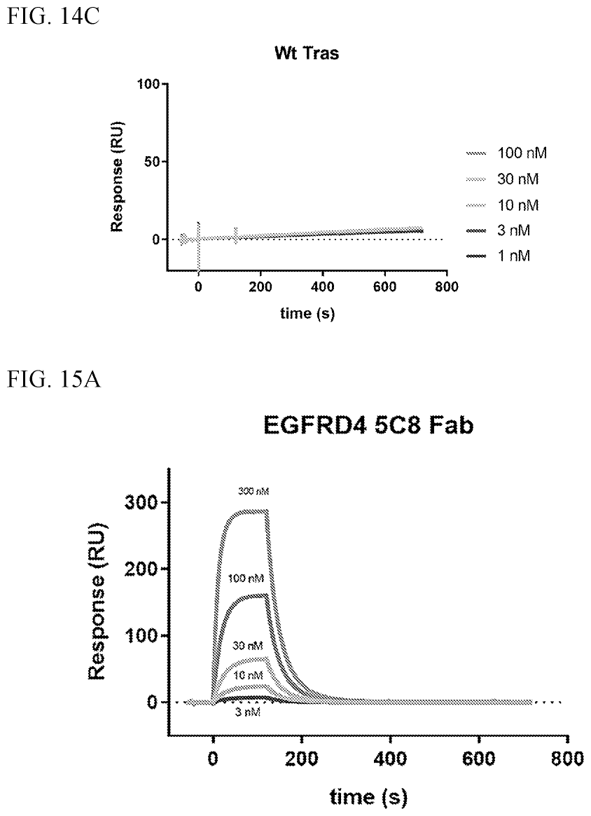

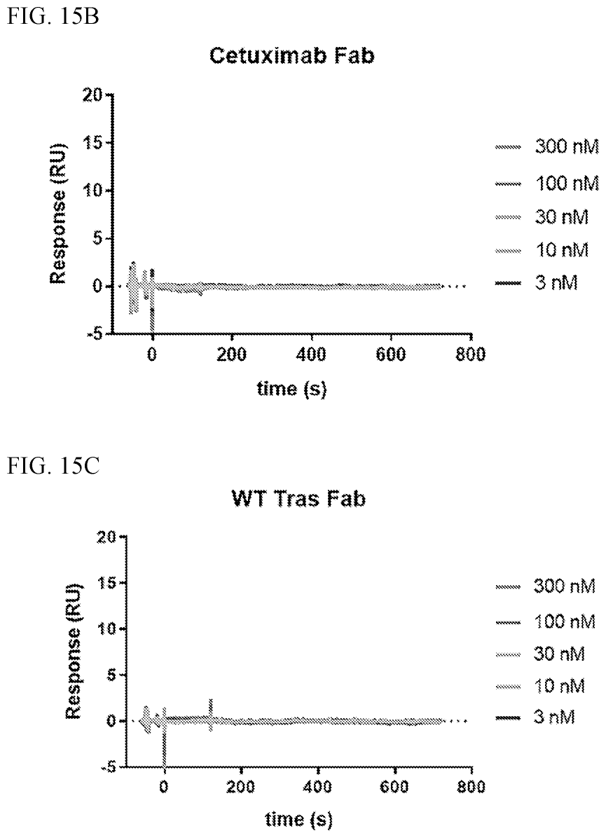

Surface plasmon resonance (SPR) experiments were conducted to characterize EGFRD4-5C8 binding to EGFR domain IV. EGFR domain IV was immobilized on a CM5 chip at a density of 500 RU through EDC/NHS coupling. EGFRD4-5C8, cetuximab Fab, and wild type traszumab (negative control) samples were prepared in HBS-EP⁺ running buffer, and were injected at concentrations of 100 nM, 30 nM, 10 nM, 3 nM, and 1 nM at 25° C. (FIGS. 14A-14C). In another experiment, EGFR domain IV was again immobilized on a CM5 chip at a density of 500 RU through EDC/NHS coupling. Instead of assessing EGFRD4-5C8 binding, the EGFRD4-5C8 Fab was tested for analyzed for binding to EGFR domain IV. EGFRD4-5C8 Fab, cetuximab Fab, and wild type traszumab samples were prepared in HBS-EP⁺ running buffer. The samples were injected at concentrations of 300 nM, 100 nM, 30 nM. 10 nM, and 3 nM (FIGS. 15A-15C). The sensorgram curves were subsequently analyzed. Comparison of 5C8 and 5C8 Fab sensorgrams show that the 5C8 IgG antibody has a relatively longer off-rate. Significantly, the faster off-rate of the Fab may be beneficial for targeting EGFR over-expressing cancer cells, while avoiding healthy cells.

### Example 4: In Vitro and In Vivo Characterization of Anti-Domain IV EGFR Antibody 5C8

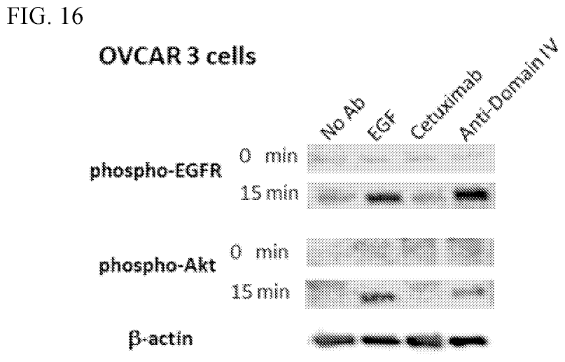

Further experiments to elucidate the mode of action of the anti-domain IV EGFR antibody clone 5C8 were conducted. The ovarian cancer cell line OVCAR3 was incubated with EGF (positive control), cetuximab, or the 5C8 antibody. Western blots were completed to detect the presence of phosphorylated EGFR, phosphorylated Akt, and β-actin (control) (FIG. 16). As expected, phosphorylation of EGFR was inhibited by cetuximab. However, 5C8 did not inhibit production of phosphorylated-EGFR confirming that 5C8 does not act by blocking EGF binding. Further, phosphorylated Akt was very weakly detected upon incubation with 5C8 and none was detected with cetuximab treatment. These results further confirm the differing modes of action of 5C8 and cetuximab.

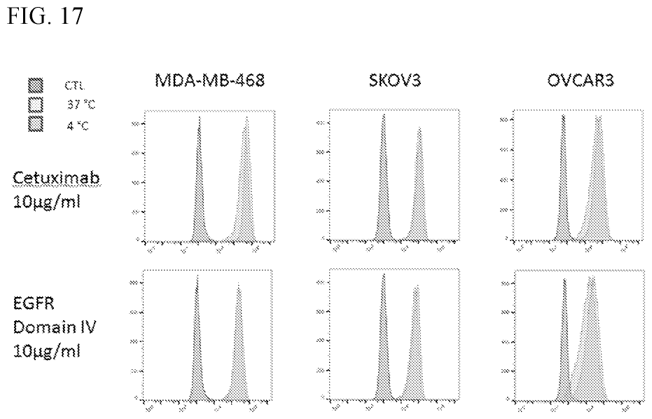

Flow cytometry experiments were completed to analyze and compare binding of EGFRD4-5C8 and cetuximab to MDA-MB-468 cells, SKOV4 cells, and OVCAR3 cells. Briefly, cells were incubated with 10 μg/mL of EGFRD4-5C8 or 10 μg/mL of cetuximab. Cells were further incubated with the secondary antibody anti-Fc PE for detection. Results indicate that both cetuximab and EGFRD4-5C8 bind to all three cell lines MDA-MB-468, SKOV4, and OVCAR3 (FIG. 17). However, the lack of significant differences in binding is attributed to fluorophore intensity and the binding of the secondary antibody.

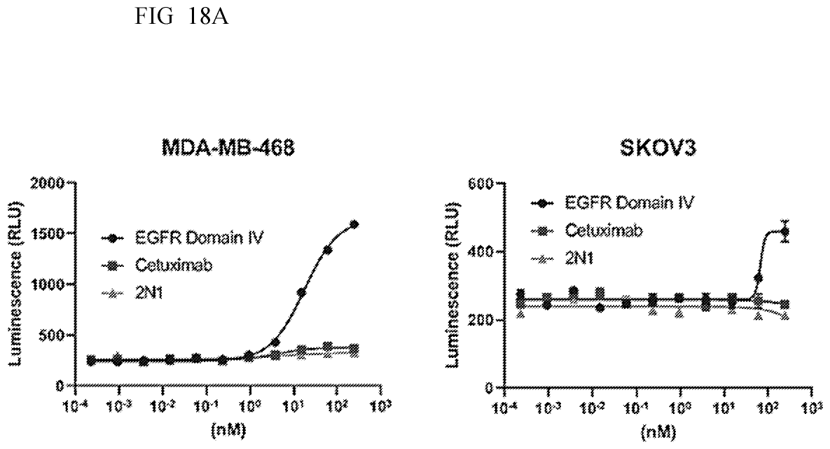

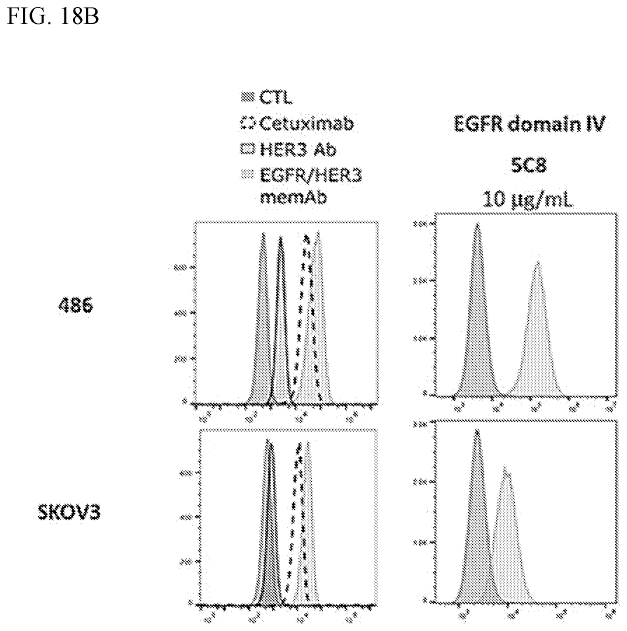

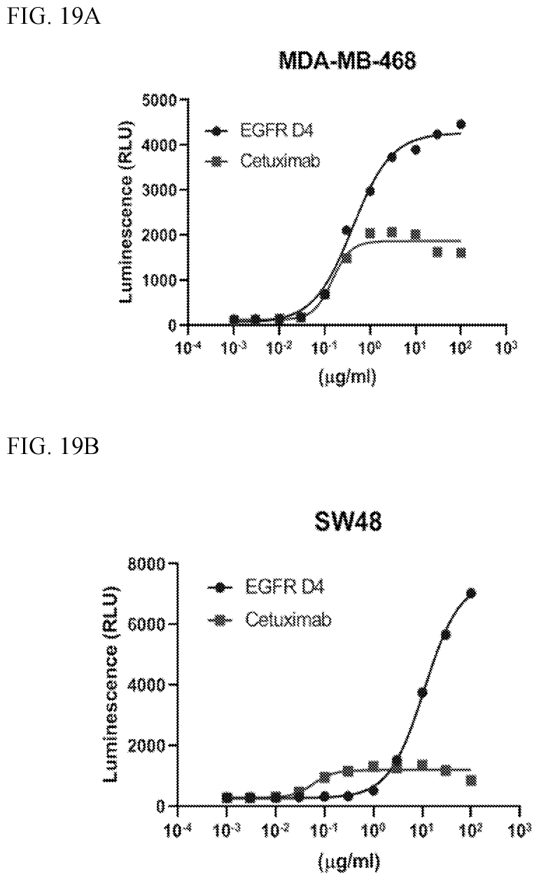

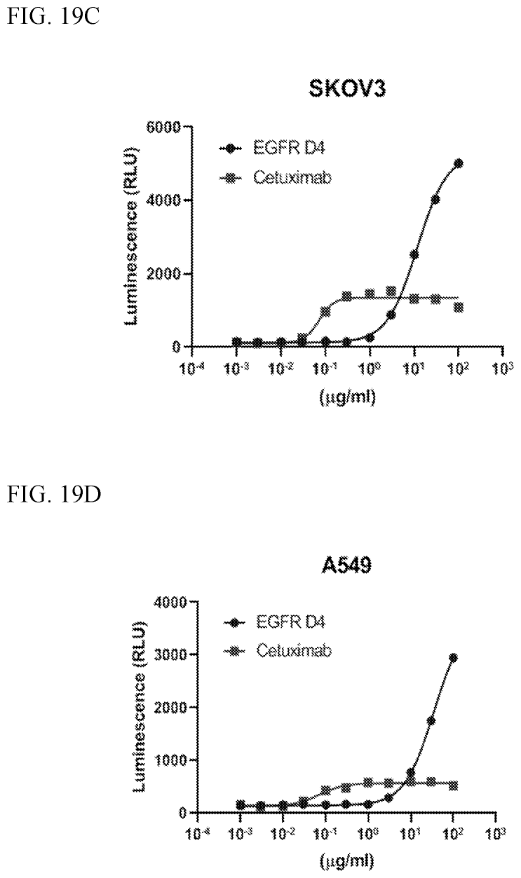

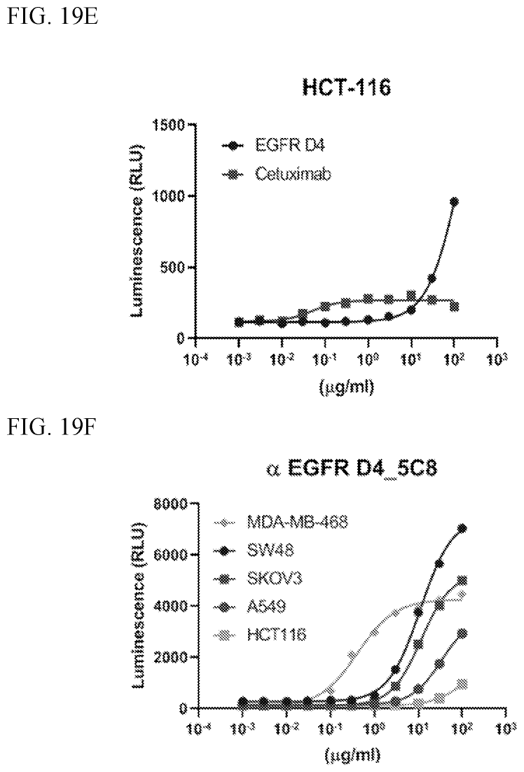

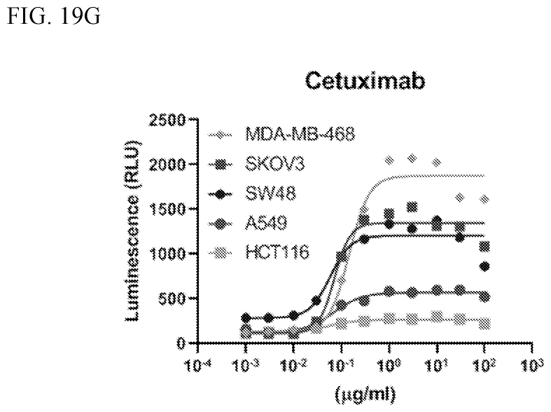

Next, initial antibody dependent cellular cytotoxicity (ADCC) induced by EGFRD4-5C8 was investigated. (FIG. 18A). EGFR-expressing cancer cell lines MDA-MB-468 and SKOV3 cells were incubated with Jurkat cells expressing NFAT-regulated luciferase and the 5C8 antibody or cetuximab at different concentrations. This system was selected due to the role of NFAT in T cell activation. After a 6 hr incubation of the cells and antibody, luciferase substrate was added in each well to react with luciferase expressed by Jurkat cells. ADCC activity was measured based on luminescence intensity. Results show that the 5C8 antibody potently activates the NFAT pathway, as shown by the increase in luminescence. Since there was not significant ADCC observed for cetuximab, a second Jurkat T cell activation readout system was created. Applicants swapped CD16 with a 158V variant, another CD16 isoform found in humans and shown to bind to the Fc with higher affinity. Once again, T cell activation was tested using the anti-domain IV EGFR 5C8 mAb and cetuximab. While cetuximab induced T cell activation was improved using the 158V variant, ADCC effects were still more subtle with cetuximab than with the EGFRd4 5C8 antibody clone (FIGS. 19A-19E). Moreover, the results show that generally, cells with higher expression levels of EGFR display greater ADCC effects (FIGS. 19F and 19G).

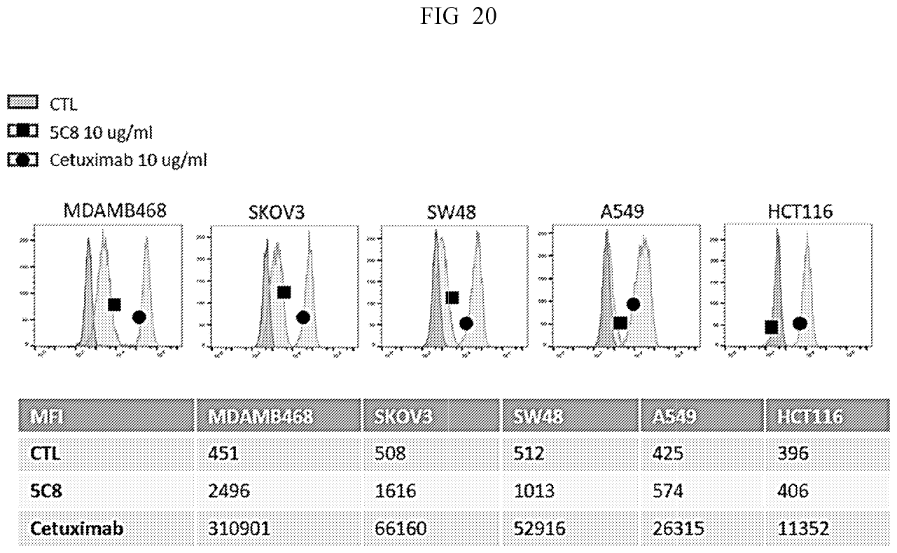

Next, EGFR expression in various cancer lines was assessed by flow cytometry. MDA-MB-468, SKOV3, SW48, A549 and HCT116 cells were incubated with 10 ug/ml 5C8 or 10 ug/ml cetuximab for 30 min and subsequently washed. Cells were stained with anti-kappa-Alexa-647 secondary antibody, and the median fluorescence intensity of each cell line before and after antibody binding was assessed (FIG. 20).

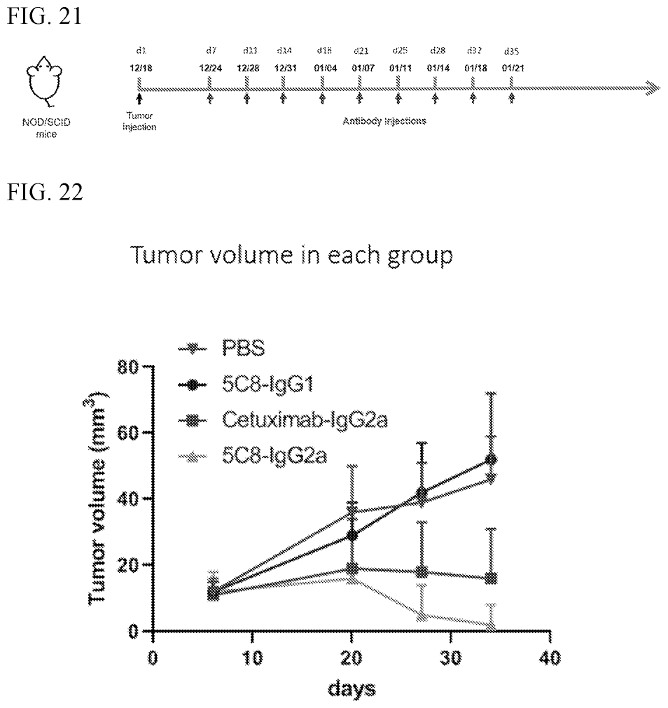

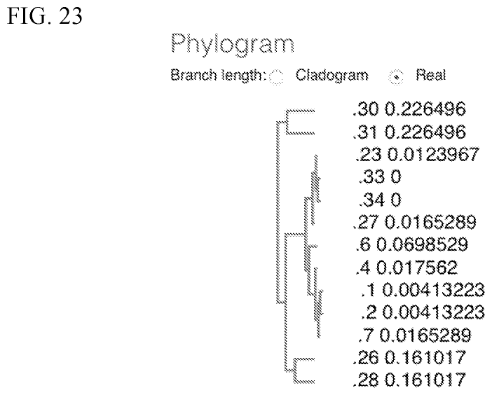

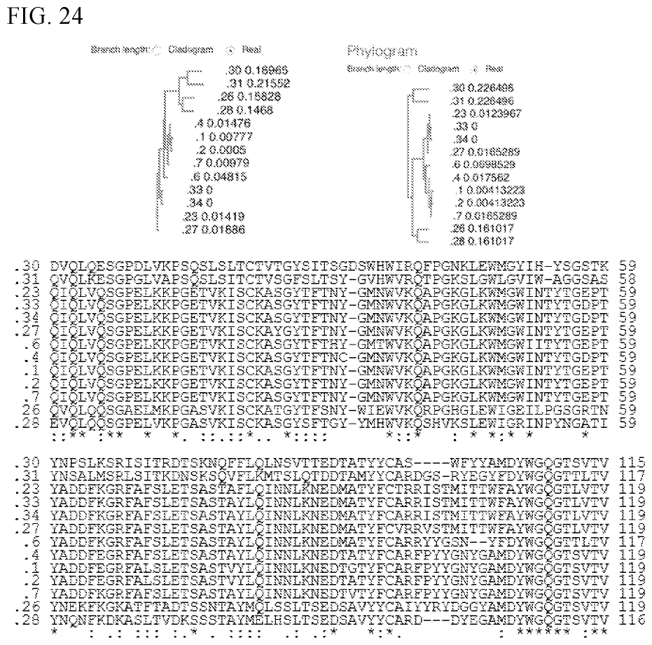

In vivo ADCC experiments using animal xenograft models were then completed (FIG. 21). Female SCID mice (BALB/c-Igh^{b scid}) were subcutaneously injected with five million MDA-MB-468 breast cancer cells on day 1. The mice were then divided into 4 groups of 5 mice for intraperitoneal administration with either 5 mg/kg PBS, 5 mg/kg 5C8-IgG1, 5 mg/kg 5C8-IgG2a, or 5 mg/kg, cetuximab-IgG2a. The weight of each mouse was approximately 20 g, thus approximately 100 ug of antibody was administered per mouse. Standard model endpoints were assessed by tumor volumes (i.e. V=(W²×L)/2 for caliper measurements) and Kaplan-Meier survival analysis. Results from the experiment show that, consistent with the mode of action, 5C8-IgG1 does not engage NK cells and thus did not have a therapeutic effect, as expected (FIG. 22). However, both cetuximab-IgG2a and 5C8-IgG2a showed potent anti-tumor effects, with 5C8-IgG2a administration resulting in the most significant decrease in tumor volume (FIG. 22).

### Example 5: Identification of Additional Anti-Domain IV EGFR Antibodies

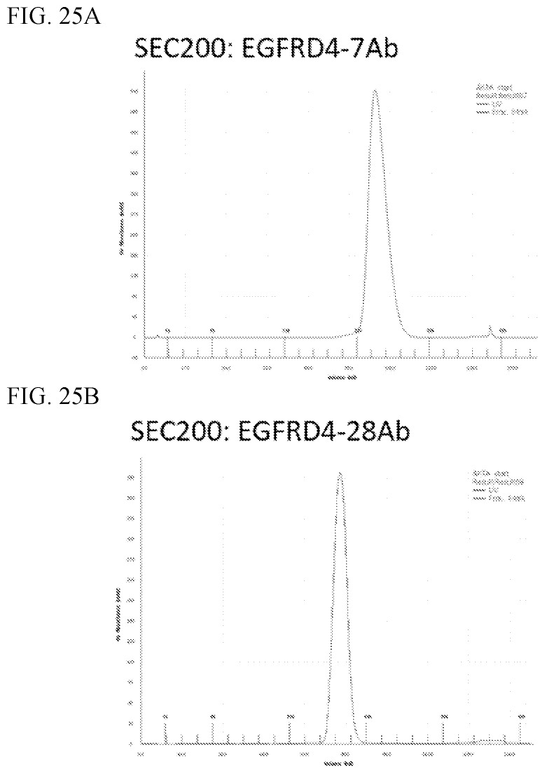

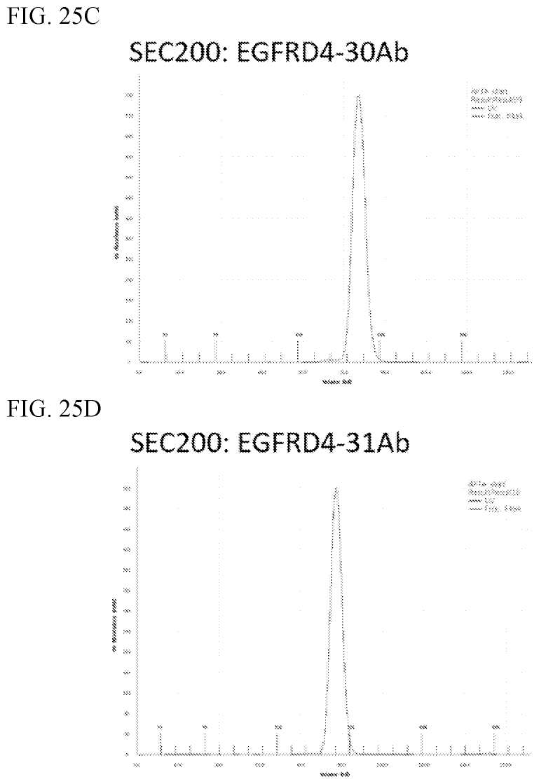

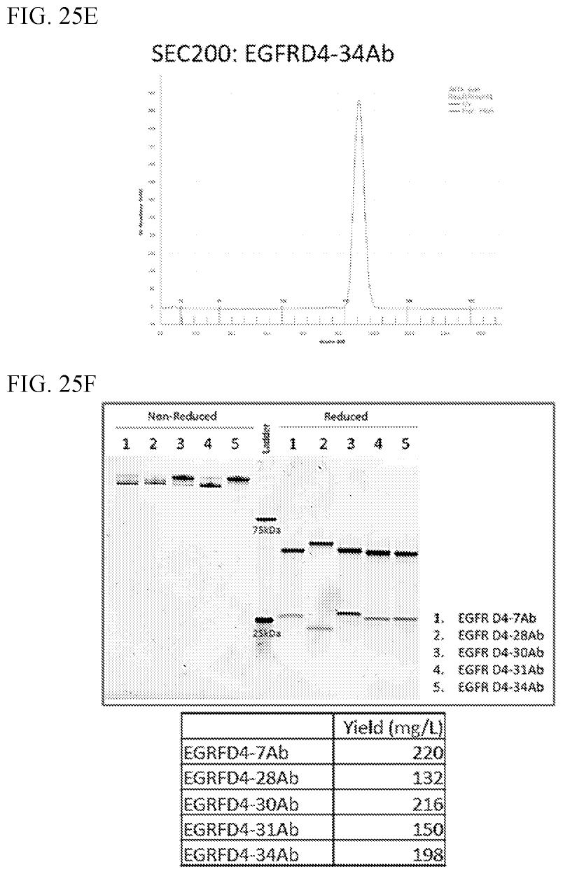

Additional antibody screening revealed further anti-domain IV EGFR antibody clones. The antibodies were purified by size exclusion chromatography and subsequently characterized by reducing and non-reducing gels (FIGS. 25A-25F). Several of the identified antibody clones (EGFRD4-7Ab, EGFRD4-28Ab, EGFRD4-30Ab, EGFRD4-31Ab, and EGFRD4-34Ab) could be expressed and purified, with the exception of EGFRD4-26.

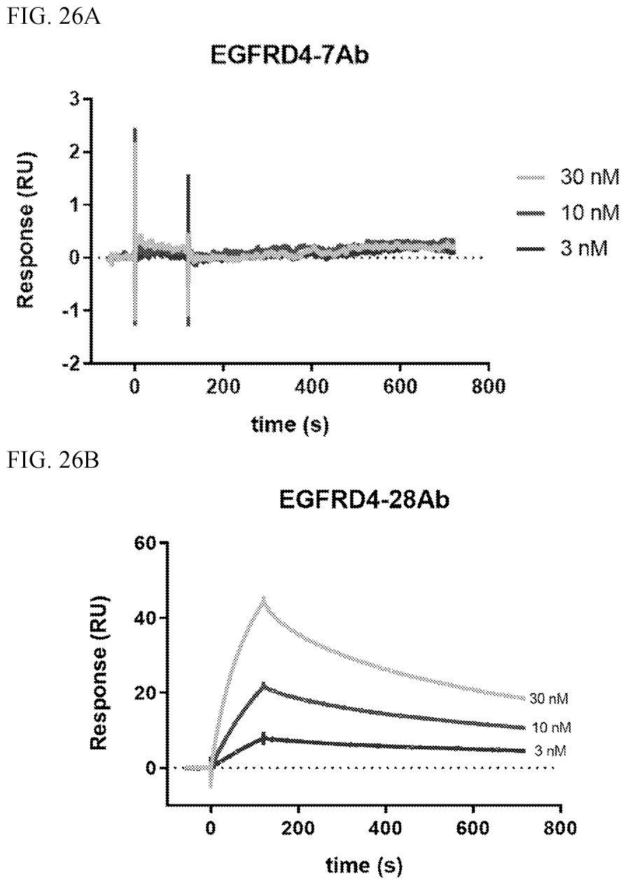

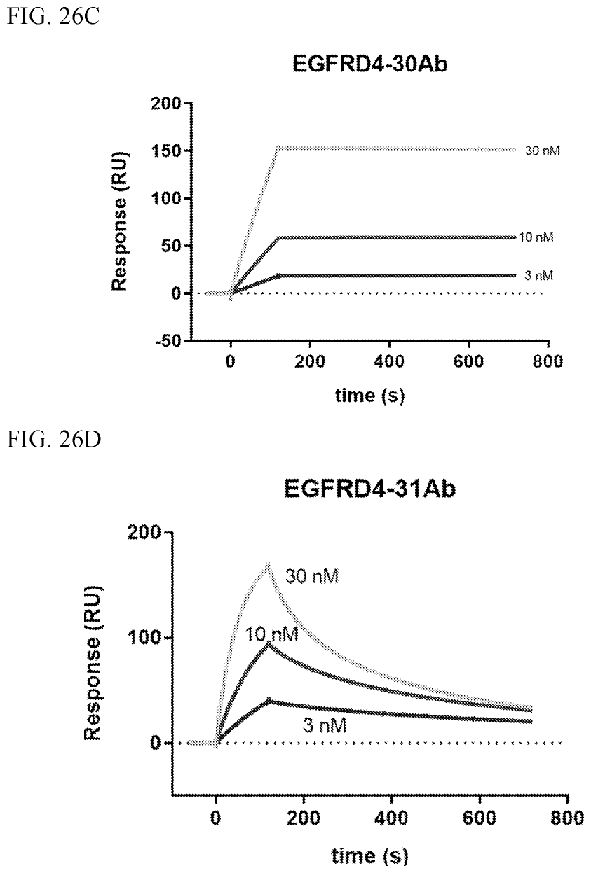

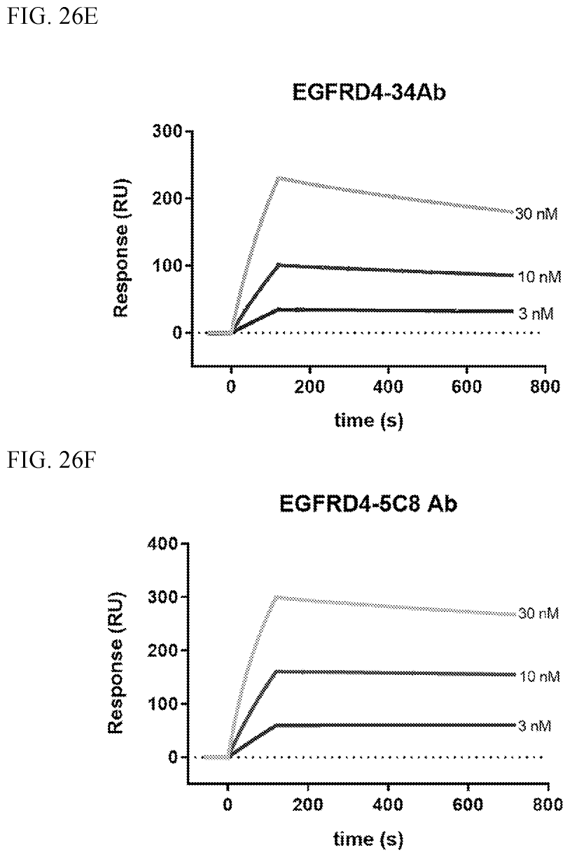

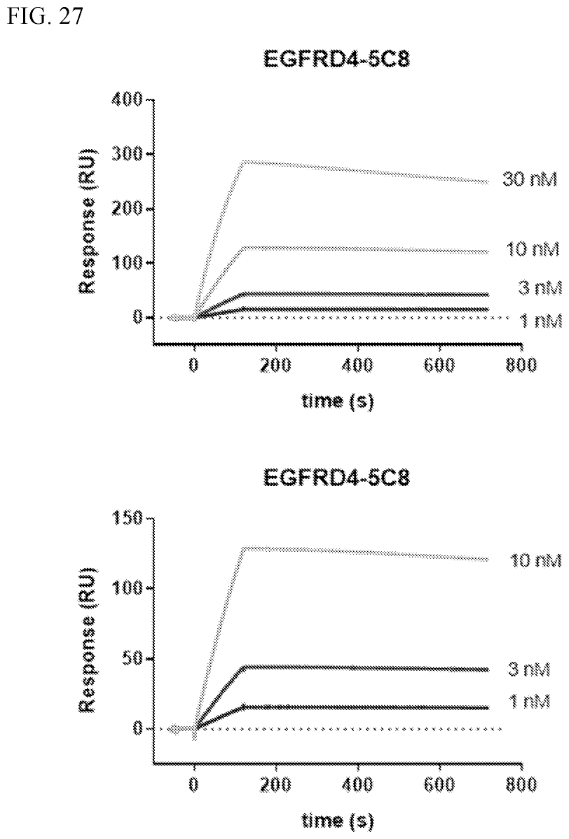

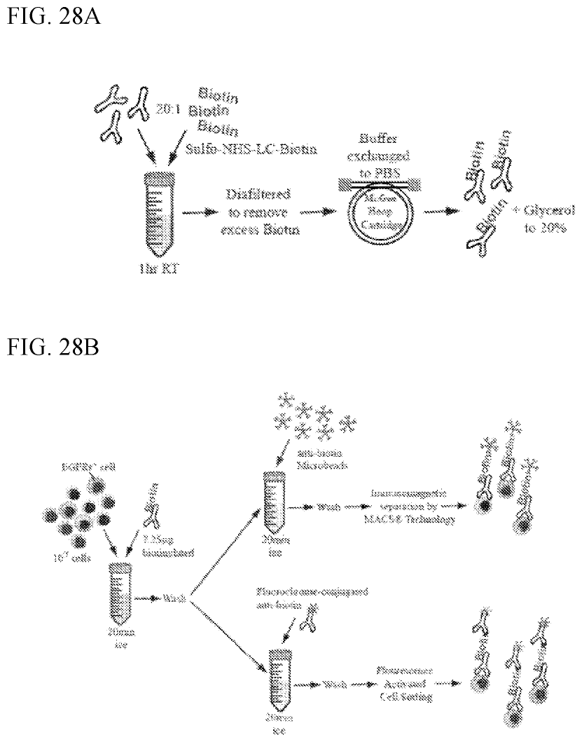

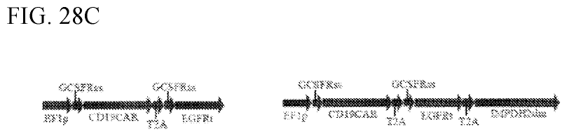

The anti-domain IV EGFR antibodies where then characterized for binding by SPR. EGFR domain IV was immobilized on an CM5 chip and various concentrations of each antibody clone were tested for binding and kinetics parameters (FIGS. 26A-26F).

The antibody clones are further assessed for ADCC both in vitro and in vivo. Further, expression is optimized for the clones, and additional members that did not express are generated. Fab domains of the clones are generated to characterize monomeric binding affinity and for crystallography with EGFR domain IV.

### Example 6: Generation of EGFRt and Immunomagnetic Selection of EGFRt Expressing T Cells

### Materials & Methods

### Antibodies and Flow Cytometry

FITC-, PE- and PerCP-conjugated isotype controls, PerCP-conjugated anti-CD8, FITC conjugated anti-CD4, PE-conjugated anti-IFN.gamma., PerCP-conjugated anti-CD45 and PE-conjugated streptavidin were obtained from BD Biosciences (San Jose, Calif.). Biotinylated anti-Fc was purchased from Jackson ImmunoResearch Laboratories, Inc. (Westgrove, Pa.). PE-conjugated anti-Biotin was purchased from Miltenyi Biotec (Auburn, Calif.). Biotinylated EGF was purchased from Molecular Probes® Invitrogen (Carlsbad, Calif.). PE-conjugated anti-EGFR was purchased from Abcam Inc. (Cambridge, Mass.). All antibodies and biotin-EGF were used according to the manufacturer's instructions. Flow cytometric data acquisition was performed on a FACScalibur (BD Biosciences), and the percentage of cells in a region of analysis was calculated using FCS Express V3 (De Novo Software, Los Angeles, Calif.).

For generation of the biotinylated-domain IV antibodies, 200 mg of an anti-domain IV EGFR antibody is buffer exchanged (19 hours) to PBS (D-PBS, pH 7.5.+−. 0.1) using a MidGee Hoop Cartridge (UFP-30-E-H42LA) with 527 mL. The material at 2 mg/mL is then modified at a 20:1 ratio using Sulfo-NHS-LC-Biotin in a reaction that is carried out for 1 hour at room temperature and then diafiltered to remove the excess biotin. The 200 mg of biotinylated anti-domain IV EGFR antibody is then buffer exchanged (18 hours) to PBS (D-PBS, pH 7.5.+−. 0.1) using MidGee Hoop Cartridge (UFP-30-E-H42LA) with 533 mL. Glycerol is added to a final concentration of 20% and then the material is frozen in vials.

### Cell Lines

Unless otherwise indicated, all cell lines are maintained in RPMI 1640 (Irvine Scientific, Santa Ana, Calif.) supplemented with 2 mM L-glutamine (Irvine Scientific), 25 mM N-2-hydroxyethylpiperazine-N′-2-ethanesulfonic acid (HEPES, Irvine Scientific), 100 U/mL penicillin, 0.1 mg/mL streptomycin (Irvine Scientific), and 10% heat-inactivated fetal calf serum (FCS, Hyclone, Logan, Utah), hereafter referred to as culture media (CM).

To generate T cells, human peripheral blood mononuclear cells (PBMC) are isolated by density gradient centrifugation over Ficoll-Paque (Pharmacia Biotech, Piscataway, N.J.) from heparinized peripheral blood obtained from consented healthy donors participating on a City of Hope National Medical Center Internal Review Board-approved protocol. For generation of Line A, washed PBMC are stimulated with 25 U/mL IL-2 and a 1:1 (cell:bead) ratio of Dynabeads® Human T expander CD3/CD28 (Invitrogen, Carlsbad, Calif.). For generation of the other lines, washed PBMC are first autoMACS™ depleted using anti-CD45RA beads (Miltenyi Biotec) per the manufacturer's protocol, and in some cases also depleted with PE-conjugated anti-CD4 (BD Biosciences) with anti-PE beads (Miltenyi Biotec). The resulting cells then undergo autoMACS™ positive selection using biotinylated DREG56 (anti-CD62L) and anti-biotin beads (Miltenyi Biotec) to produce purified CD62+CD45RO+ T^{CM} CD8+ cells are further selected in some cases using AutoMACS™ (Miltenyi Biotec) per the manufacturer's protocol. CMV-specific cells are generated by stimulating T cells with 5 U/ml rhIL-2 (Chiron, Emeryville, Calif.) and autologous irradiated viral antigen presenting cells at a 4:1 (responder:stimulator) ratio once a week for three weeks, using 10% human serum instead of FCS to avoid non-specific stimulation. The viral antigen presenting cells are derived from PBMC that had been genetically modified to express CMVpp65 antigen.

PBMC are resuspended in nucleofection solution using the Human T cell Nucleofector kit (Amaxa Inc., Gaithersberg, Md.), and 5×10⁷ cells are aliquoted into 0.2-cm cuvettes containing 10 ug HygroR-pp 65_pEK (or pmaxGFP from Amaxa Inc., as a transfection control) in a final volume of 100 uL/cuvette, and electroporated using the Amaxa Nucleofector I (Amaxa Inc.), program U-14, after which cells are allowed to recover for 6 hours at 37° C. prior to γ-irradiation (1200 cGy).

The CD19CAR-T2A-EGFRt_epHIV7 (pJ02104) and CD19CAR-T2A-EGFRt-T2A-IMPDH2dm_epHIV7 (pJ02111) lentiviral constructs include a) the chimeric antigen receptor (CAR) sequences including the VH and VL gene segments of the CD19-specific FmC63 mAb, an IgG1 hinge-C_{H2}-C_{H3}, the transmembrane and cytoplasmic signaling domains of the costimulatory molecule CD28, and the cytoplasmic domain of the CD3 zeta chain [10]; b) the self-cleaving T2A sequence [11]; c) the tEGFR surface molecule including the amino acid sequence of SEQ ID NO:276 (See FIG. 1) and a transmembrane portion (tEGFR transmembrane domain having, e.g., SEQ ID NO:283); and d) the IMPDH2 double mutant that confers MPA-resistance, as indicated. Lentiviral transduction is carried out on T cells that are stimulated with either 30 ng/ml anti-CD3.epsilon. (OKT3: Ortho Biotech, Raritan, N.J.) (i.e., for Line A) or human CD3/CD28Dynal beads at a 1:10 ratio (i.e., for Lines B, C, D and E) and 25 U IL2/ml. Cells are cultured for up to 2 hours at 37° C. on RetroNectin® (50) ug/ml) coated plates prior to addition of the lentivirus at an MOI of 3 and 5 g/ml polyybrene. After 4 hours, warm medium is added to triple to volume, and the cells are then washed and plated in fresh media after 48 hours. AutoMACS™ sorting of EGFRt-expressing cells is carried out with biotinylated anti-domain IV EGFR antibody provided herein and anti-biotin microbeads (Miltenyi Biotec) as per the manufacturer's instructions. Expansion of T cells in rapid expansion medium (REM) involves the incubation of 10⁶ T cells with 30 ng/ml anti-CD38. (OKT3: Ortho Biotech, Raritan, N.J.), 5×10⁷.gamma.-irradiated PBMCs (3500 cGy), and 10×10⁷.gamma.-irradiated LCLs (8000 cGy) in 50 mL CM; with addition of 50 U/mL rhIL-2 and 10 ng/ml rhIL-15 (CellGenix) every 48 hours, beginning on day 1. T cells are re-stimulated in this manner every 14 days.

EBV-transformed lymphoblastoid cell lines (LCLs) are made from PBMC as previously described [13]. LCL-OKT3 cells are generated by resuspending LCL in nucleofection solution using the Amaxa Nucleofector kit T, adding OKT3-2A-Hygromycin_pEK (pJ01609) plasmid at 5 ug/10×10⁷ cells, and electroporating cells using the Amaxa Nucleofector I, program T-20. The resulting LCL-OKT3-2A-Hygro_pEK (cJ03987) are grown in CM containing 0.4 mg/ml hygromycin. The mouse myeloma line NS0 (gift from Andrew Raubitschek, City of Hope National Medical Center, Duarte, Calif.) is resuspended in nucleofection solution using the Nucleofector kit T (Amaxa Inc., Gaithersberg, Md.), CD19t-DHFRdm-2A-IL12_pEK (pJ01607) or GFP-IMPDH2dm-2A-IL15_pcDNA3.1 (+) (pJ01043) plasmid is added at 5 ug/5×10⁶ cells, and cells are electroporated using the Amaxa Nucleofector I, program T-27. The resulting NS0-CD19t-DHFRdm-2A-IL12_pEK (cJ03935) and NS0-GFP:IMPDH2-IL15 (IL2ss)_pcDNA3.1 (+) (cJ02096) are grown in DMEM (Irvine Scientific, Santa Ana, Calif.) supplemented with 10% heat-inactivated FCS, 25 mM HEPES, and 2 mM L-glutamine in the presence of either 0.05 uM methotrexate (MTX) or 6 μM mycophenolic acid (MPA). U251T-pp 65 are generated by lentiviral transduction of U251T with pp 65-2A-eGFP-ffluc_epHIV7 (pJ01928) at an MOI of 1. The resulting U251T-pp 65-2A-eGFP-ffluc_epHIV7 are then FACS sorted for the GFP+ population (cJ05058). The Daudi lymphoma line is purchased from ATCC and grown in media consisting of RPMI 1640 (Irvine Scientific), 2 mM L-Glutamine (Irvine Scientific), 10% heat-inactivated FCS (Hyclone) B15 acute lymphoblastic leukemia cells and A431 epidermoid carcinoma cells are purchased from ATCC.

### Protein Analysis

Cells (up to 10⁷) are lysed with 80 μL of 1% Triton-X lysis buffer containing phosphatase inhibitor cocktail II (Sigma-Aldrich Corp., St. Louis, Mo.) (1:20 of inhibitor to buffer by volume). 50 μg of protein is loaded in each lane, and Western blots are probed with antibodies from the Phospho-EGF receptor antibody sampler kit (Cell Signaling Technology, Inc., Danvers, Mass.) followed by IRDye™ 680CW or 800CW conjugated goat anti-rabbit antibodies (LI-COR, Lincoln, Nebr.), as well as the IRDye™ 800 conjugated anti-beta-Actin antibody (LI-COR) as per the manufacturers' instructions. Blots are imaged on the Odyssey Infrared Imaging System (LI-COR).

### Chromium-Release Assays

The cytolytic activity of T cells is determined by 4-hour chromium-release assay (CRA), where effector cells are seeded into triplicate wells of V-bottom 96-well micro-plates containing 5×10³ ₅₁Cr-labeled target cells (Na_{2 51}CrO₄; (5 mCi/mL); Amersham Pharmacia, Piscataway, N.J.) at various E:T ratios in 200 μL of CM and incubated for 4 hours at 5% CO₂, 37° C. Plates are centrifuged, and 100 ul of supernatant is removed from each well to assess chromium release using a .gamma.-counter (Packard Cobra II, Downer's Grove, Ill.). The percent specific lysis is calculated as follows: 100× (experimental release-spontaneous release)/(maximum release-spontaneous release). Maximum release is determined by measuring the ⁵¹Cr content of wells containing labeled targets lysed with 2% SDS.

Antibody dependent cell mediated cytotoxicity is determined by chromium release as above using 5×10³ ₅₁Cr-labeled target cells that have been pre-incubated for 90 min with up to 10 μg/mL of anti-domain IV EGFR antibody, washed and then co-incubated with 5×10⁵ freshly isolated PBMC.

### T Cell Engraftment and Anti-Domain IV EGFR Antibody Mediated Suicide In Vivo

For T cell engraftment, six- to ten-week old NOD/Scid IL-2R.gamma.C.sup.null mice are injected i.v. on day 0 with 10⁷ T cells. 2×10⁷ irradiated (8000 rads) NS0-GFP:IMPDH2-IL15 (IL2ss)_pcDNA3.1 (+) (cJ02096) cells are administered i.p. 3 times a week starting on day 0 to provide a systemic supply of human IL-15 in vivo. Bone marrow is harvested from euthanized animals and analyzed by flow cytometry. Antibody dependent cell mediated cytotoxicity assays are performed to determine the activity of anti-domain IV EGFR antibody against EGFRt+T cells.

To immunomagnetically select for EGFRt-expressing cells, biotinylated-anti-domain IV EGFR antibody is generated to be used in conjunction with commercially available anti-biotin microbeads and an AutoMACS™ separator (Miltenyi Biotec) (FIG. 2c). Lentiviral transduction of various T cell lines with EGFRt-containing constructs, where the EGFRt gene is separated from other genes of interest on either one or both ends with the self-cleaving T2A sequence are generated. Surface detection may be accomplished with a EGFRt-sr39TK fusion. Immunomagnetic selection will allow for recovery of EGFRt+ T cell populations with greater than 90% purity.

### Example 7: Therapeutic Use of EGFRT+ T Cells

Adult subjects with high-risk intermediate grade B-cell lymphomas who are candidates for an autologous myeloablative stem cell transplant procedure may receive post-transplant immunotherapy with adoptively transferred autologous Tcm-derived CD19R+ CD8+ EGFRt+ T cell grafts. A leukapheresis product collected from each patient undergoes selection of Tcm, transduction with clinical grade CD19CAR-T2A-EGFRt_epHIV7, and then selection and expansion of the EGFRt+ cells in a closed system. After the resulting cell products have undergone quality control testing (including sterility and tumor specific cytotoxicity tests), they are cryopreserved. Meanwhile, following leukapheresis, study participants commence with standard salvage chemotherapy, with mobilization for auto HSC collection with cytoreductive chemotherapy and G-CSF.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All patents, patent applications, and references cited throughout the specification are expressly incorporated by reference.

### REFERENCES

1. Berger, C, Flowers, M E, Warren, E H, and Riddell, S R (2006). Analysis of transgene-specific immune responses that limit the in vivo persistence of adoptively transferred HSV-TK-modified donor T cells after allogeneic hematopoietic cell transplantation. Blood 107: 2294-302.
2. Tey, S K, Dotti, G, Rooney, C M, Heslop, H E, and Brenner, M K (2007). Inducible caspase 9 suicide gene to improve the safety of allodepleted T cells after haploidentical stem cell transplantation. Biol Blood Marrow Transplant 13:913-24.
3. Fehse, B, Richters, A, Putimtseva-Scharf, K, Klump, H, Li, Z, Ostertag, W, et al. (2000). CD34 splice variant: an attractive marker for selection of gene-modified cells. Mol Ther 1:448-56.
4. Gaines, P, and Wojchowski, D M (1999). pIRES-CD4t, a dicistronic expression vector for MACS- or FACS-based selection of transfected cells. Biotechniques 26:683-8.
5. Fehse, B, Uhde, A, Fehse, N, Eckert, H G, Clausen, J, Ruger, R, et al. (1997). Selective immunoaffinity-based enrichment of CD34+ cells transduced with retroviral vectors containing an intracytoplasmatically truncated version of the human low-affinity nerve growth factor receptor (deltaLNGFR) gene. Hum Gene Ther 8:1815-24.
6. Lemoine, F M, Mesel-Lemoine, M, Cherai, M, Gallot, G, Vie, H, Leclercq, V, et al. (2004). Efficient transduction and selection of human T-lymphocytes with bicistronic Thy 1/HSV1-TK retroviral vector produced by a human packaging cell line. J Gene Med 6:374-86.
7. Li, S, Schmitz, K R, Jeffrey, P D, Wiltzius, J J, Kussie, P, and Ferguson, K M (2005). Structural basis for inhibition of the epidermal growth factor receptor by cetuximab. Cancer Cell 7:301-11.
8. Dawson, J P, Berger, M B, Lin, C C, Schlessinger, J, Lemmon, M A, and Ferguson, K M (2005). Epidermal growth factor receptor dimerization and activation require ligand-induced conformational changes in the dimer interface. Mol Cell Biol 25:7734-42.
9. Lange, C, Li, Z, Fang, L, Baum, C, and Fehse, B (2007). CD34 modulates the trafficking behavior of hematopoietic cells in vivo. Stem Cells Dev 16:297-304.
10. Kowolik, C M, Topp, M S, Gonzalez, S, Pfeiffer, T, Olivares, S, Gonzalez, N, et al. (2006). CD28 costimulation provided through a CD19-specific chimeric antigen receptor enhances in vivo persistence and antitumor efficacy of adoptively transferred T cells. Cancer Res 66:10995-1004.
11. Szymczak, A L, Workman, C J, Wang, Y, Vignali, K M, Dilioglou, S, Vanin, E F, et al. (2004). Correction of multi-gene deficiency in vivo using a single ‘self-cleaving’ 2A peptide-based retroviral vector. Nat Biotechnol 22:589-94.
12. Yam, P, Jensen, M, Akkina, R, Anderson, J, Villacres, M C, Wu, J, et al. (2006). Ex vivo selection and expansion of cells based on expression of a mutated inosine monophosphate dehydrogenase 2 after HIV vector transduction: effects on lymphocytes, monocytes, and CD34+ stem cells. Mol Ther 14:236-44.
13. Pelloquin, F, Lamelin, J P, and Lenoir, G M (1986). Human B lymphocytes immortalization by Epstein-Barr virus in the presence of cyclosporin A. In Vitro Cell Dev Biol 22:689-94.

INFORMAL SEQUENCE LISTING
(D4-IgG2a)
SEQ ID NO: 273
NHVCHALCSPEGCWGPEPRDCVSCRNVSRGRECVDKCNLLEGEPR
EFVENSECIQCHPECLPQAMNITCTGRGPDNCIQCAHYIDGPHCV
KTCPAGVMGENNTLVWKYADAGHVCHLCHPNCTYGCTGPGLEGCP
TNGPKIPSGGGSGGGSKPCPPCKCPAPNLLGGPSVFIFPPKIKDV
LMISLSPIVTCVVVDVSEDDPDVQISWFVNNVEVHTAQTQTHRED
YNSTLRVVSALPIQHQDWMSGKEFKCKVNNKDLPAPIERTISKPK
GSVRAPQVYVLPPPEEEMTKKQVTLTCMVTDFMPEDIYVEWTNNG
KTELNYKNTEPVLDSDGSYFMYSKLRVEKKNWVERNSYSCSVVHE
GLHNHHTTKSFSRTPGK
(EGFR-D4-IgG2a)
SEQ ID NO: 274
PKCDPSCPNGSCWGGGEENCQKLTKIICAQQCSHRCRGRSPSDCC
HNQCAAGCTGPRESDCLVCQKFQDEATCKDTCPPLMLYNPTTYQM
DVNPEGKYSFGATCVKKCPRNYVVTDHGSCVRACGPDYYEVEEDG
IRKCKKCDGPCRKVCNGIGIGEFKDTLSINATNIKHFKYCTAISG
DLHILPVAFKGDSFTRTPPLDPRELEILKTVKEITGFLLIQAWPD
NWTDLHAFENLEIIRGRTKQHGQFSLAVVGLNITSLGLRSLKEIS
DGDVIISGNRNLCYANTINWKKLFGTPNQKTKIMNNRAEKDCKAV
NHVCHALCSPEGCWGPEPRDCVSCRNVSRGRECVDKCNLLEGEPR
EFVENSECIQCHPECLPQAMNITCTGRGPDNCIQCAHYIDGPHCV
KTCPAGVMGENNTLVWKYADAGHVCHLCHPNCTYGCTGPGLEGCP
TNGPKIPSGGGSGGGSKPCPPCKCPAPNLLGGPSVFIFPPKIKDV
LMISLSPIVTCVVVDVSEDDPDVQISWFVNNVEVHTAQTQTHRED
YNSTLRVVSALPIQHQDWMSGKEFKCKVNNKDLPAPIERTISKPK
GSVRAPQVYVLPPPEEEMTKKQVTLTCMVTDFMPEDIYVEWTNNG
KTELNYKNTEPVLDSDGSYFMYSKLRVEKKNWVERNSYSCSVVHE
GLHNHHTTKSFSRTPGK
(D4-IgA)
SEQ ID NO: 275
GQVCHALCSPEGCWGPEPRDCVSCRNVSRGRECVDKCNLLEGEPR
EFVENSECIQCHPECLPQAMNITCTGRGPDNCIQCAHYIDGPHCV
KTCPAGVMGENNTLVWKYADAGHVCHLCHPNCTYGCTGPGLEGCP
TNGPKIPSVPSTPPTPSPSTPPTPSPSCCHPRLSLHRPALEDLLL
GSEANLTCTLTGLRDASGVTFTWTPSSGKSAVQGPPERDLCGCYS
VSSVLPGCAEPWNHGKTFTCTAAYPESKTPLTATLSKSGNTFRPE
VHLLPPPSEELALNELVTLTCLARGFSPKDVLVRWLQGSQELPRE
KYLTWASRQEPSQGTTTFAVTSILRVAAEDWKKGDTFSCMVGHEA
LPLAFTQKTIDRLAGKPTHVNVSVVMAEVDGTSY
(tEGFR domain IV)
SEQ ID NO: 276
VCHALCSPEGCWGPEPRDCVSCRNVSRGRECVDKCNLLEGEPREF
VENSECIQCHPECLPQAMNITCTGRGPDNCIQCAHYIDGPHCVKT
CPAGVMGENNTLVWKYADAGHVCHLCHPNCTYGCTGPGLEGCPTN
GPKIPS
(tEGFR transmembrane domain)
SEQ ID NO: 283
IATGMVGALLLLLVVALGIGLFM
(tEGFR signal peptide)
SEQ ID NO: 284
MLLLVTSLLLCELPHPAFLLIP
(tEGFR domain IV plus tEGFR transmembrane domain)
SEQ ID NO: 285
VCHALCSPEGCWGPEPRDCVSCRNVSRGRECVDKCNLLEGEPREF
VENSECIQCHPECLPQAMNITCTGRGPDNCIQCAHYIDGPHCVKT
CPAGVMGENNTLVWKYADAGHVCHLCHPNCTYGCTGPGLEGCPTN
GPKIPSIATGMVGALLLLLVVALGIGLFM

TABLE 1
CDR sequences of exemplary antibody heavy chains.

| Clone | SEQ | | | |
|---|---|---|---|---|
| # | ID NO | | VH | Screening |
| 1 | 1. | CDR1 | GYTFTNY | EGFR-D4 |
| | 2. | CDR2 | NTYTGE | |
| | 3. | CDR3 | FPYYGNYGAMDY | |
| 2 | 4. | CDR1 | GYTFTNY | EGFR-D4 |
| | 5. | CDR2 | NTYTGE | |
| | 6. | CDR3 | FPYYGNYGAMDY | |
| 4 | 7. | CDR1 | GYTFTNY | EGFR-D4 |
| | 8. | CDR2 | NPYTGE | |
| | 9. | CDR3 | SVYGYWYFDV | |
| 5 | 10. | CDR1 | GYTFTNC | EGFR-D4 |
| | 11. | CDR2 | NTYTGD | |
| | 12. | CDR3 | FPYYGNYGAMDY | |
| 6 | 13. | CDR1 | GYTFTHY | EGFR-D4 |
| | 14. | CDR2 | ITYTGE | |
| | 15. | CDR3 | YYGSNYFDY | |
| 7 | 16. | CDR1 | GYTFTNY | EGFR-D4 |
| | 17. | CDR2 | INTYTGE | |
| | 18. | CDR3 | FPYYGNYGAMDY | |
| 8 | 19. | CDR1 | GFSLSTSGM | EGFR |
| | 20. | CDR2 | YWDDD | |
| | 21. | CDR3 | RGNYYAMDY | |
| 9 | 22. | CDR1 | GYTFTSY | EGFR |
| | 23. | CDR2 | NPSNGR | |
| | 24. | CDR3 | YYDNDDFDY | |
| 11 | 25. | CDR1 | GYTFTNY | EGFR |
| | 26. | CDR2 | NTYTGE | |
| | 27. | CDR3 | GGYYRYALY | |
| 12 | 28. | CDR1 | KASGYTFTSY | EGFR |
| | 29. | CDR2 | NPSNGR | |
| | 30. | CDR3 | YYDNDDFDY | |
| 14 | 31. | CDR1 | GYTFTNY | EGFR |
| | 32. | CDR2 | NPYTGE | |
| | 33. | CDR3 | SVYGYWYLDV | |
| 15 | 34. | CDR1 | GYSITSDY | EGFR |
| | 35. | CDR2 | SYSGN | |
| | 36. | CDR3 | AATGYVMDY | |

TABLE 2
CDR sequences of exemplary antibody light chains.

| Clone | SEQ | | | |
|---|---|---|---|---|
| # | ID NO | | VL | Screening |
| 1 | 37. | CDR1 | RSSKSLLNSNGINYLY | EGFR-D4 |
| | 38. | CDR2 | QMSNLAS | |
| | 39. | CDR3 | AQNLELPCT | |
| 2 | 40. | CDR1 | KANKDGNNNLN | EGFR-D4 |
| | 41. | CDR2 | GASNLES | |
| | 42. | CDR3 | QQNSNFPYT | |
| 4 | 43. | CDR1 | ND | EGFR-D4 |
| | 44 | CDR2 | | |
| | 45 | CDR3 | | |
| 5 | 46. | CDR1 | RSSKSLLHSNGITYL | EGFR-D4 |
| | 47. | CDR2 | QMSNLAS | |
| | 48. | CDR3 | AQNLELPWT | |
| 6 | 49. | CDR1 | RASESVDSYGNSFM | EGFR-D4 |
| | 50. | CDR2 | RASNLES | |
| | 51. | CDR3 | QQSNEDPYT | |
| 7 | 52. | CDR1 | RSSKSLLHSNGVTYL | EGFR-D4 |
| | 53. | CDR2 | QMSNLAS | |
| | 54 | CDR3 | AQNLELPCT | |
| 8 | 55. | CDR1 | SASSSVSYM | EGFR |
| | 56. | CDR2 | LTSILAS | |
| | 57. | CDR3 | QQWSSYPPT | |
| 9 | 58. | CDR1 | KSSQSLLDSDGKTYLN | EGFR |
| | 59. | CDR2 | LVSKLDS | |
| | 60. | CDR3 | WQGTHFPHT | |
| 11 | 61 | CDR1 | KASEDIDNRLG | EGFR |
| | 62 | CDR2 | GATSLET | |
| | 63. | CDR3 | QQYWSTPYT | |
| 12 | 64. | CDR1 | ND | EGFR |
| | 65. | CDR2 | | |
| | 66. | CDR3 | | |
| 14 | 67. | CDR1 | ND | EGFR |
| | 68. | CDR2 | | |
| | 69. | CDR3 | | |
| 15 | 70. | CDR1 | SASSSVSYMN | EGFR |
| | 71. | CDR2 | GISYLAS | |
| | 72. | CDR3 | QQRSSYPLT | |

TABLE 3
CDR sequences of exemplary antibody heavy chains.

| Clone | SEQ | | | |
|---|---|---|---|---|
| # | ID NO | | VH | Screening |
| 16 | 73. | CDR1 | GYTFTNY | EGFR |
| | 74. | CDR2 | NTYTGE | |
| | 75. | CDR3 | FPYYGNYGAMDY | |
| 17 | 76. | CDR1 | ND | EGFR |
| | 77. | CDR2 | | |
| | 78. | CDR3 | | |
| 18 | 79. | CDR1 | GYTFTDY | EGFR |
| | 80. | CDR2 | DPETGG | |
| | 81. | CDR3 | AVVATDYFD | |
| 19 | 82. | CDR1 | ND | EGFR |
| | 83. | CDR2 | | |
| | 84. | CDR3 | | |
| 20 | 85. | CDR1 | GFNIKDT | EGFR |
| | 86. | CDR2 | DPATGN | |
| | 87. | CDR3 | HYYGGTYYPMDY | |
| 23 | 88. | CDR1 | GYTFTNY | EGFR-D4 |
| | 89. | CDR2 | NTYTGE | |
| | 90. | CDR3 | RISTMITTWFAY | |
| 24 | 91. | CDR1 | GFSFTGY | EGFR-D4 |
| | 92. | CDR2 | NPYNGV | |
| | 93. | CDR3 | LDYDSSFDY | |
| 26 | 94. | CDR1 | GYTFSNY | EGFR-D4 |
| | 95. | CDR2 | LPGSGR | |
| | 96. | CDR3 | YYRYDGGYAMDYW | |
| 27 | 97. | CDR1 | YGYTFTNY | EGFR-D4 |
| | 98. | CDR2 | NTYTGE | |
| | 99. | CDR3 | VRRVSTMITTWFAY | |
| 28 | 100. | CDR1 | GYSFTGY | EGFR-D4 |
| | 101. | CDR2 | NPYNGA | |
| | 102. | CDR3 | DDYEGAMDY | |
| 30 | 103. | CDR1 | GYSITSGD | EGFR-D4 |
| | 104. | CDR2 | HYSGS | |
| | 105. | CDR3 | WFYYAMDY | |

TABLE 4
CDR sequences of exemplary antibody light chains.

| Clone | SEQ | | | |
|---|---|---|---|---|
| # | ID NO | | VL | Screening |
| 16 | 106. | CDR1 | ND | EGFR |
| | 107. | CDR2 | | |
| | 108. | CDR3 | | |
| 17 | 109. | CDR1 | KASQNVGTNVA | EGFR |
| | 110. | CDR2 | SASYRYS | |
| | 111. | CDR3 | QYNSYPYT | |
| 18 | 112. | CDR1 | ND | EGFR |
| | 113. | CDR2 | | |
| | 114. | CDR3 | | |
| 19 | 115. | CDR1 | QDINKYIA | EGFR |
| | 116. | CDR2 | YTSTLQP | |
| | 117. | CDR3 | LQYDNLWT | |
| 20 | 118. | CDR1 | KSSQSLLNSRTRKNYLA | EGFR |
| | 119. | CDR2 | WASTRES | |
| | 120. | CDR3 | KQSYNLWT | |
| 23 | 121. | CDR1 | RASENIYSYLA | EGFR-D4 |
| | 122. | CDR2 | YAKTLAE | |
| | 123. | CDR3 | QHHYGTPYT | |
| 24 | 124. | CDR1 | ND | EGFR-D4 |
| | 125. | CDR2 | | |
| | 126. | CDR3 | | |
| 26 | 127. | CDR1 | RASQEISGYLN | EGFR-D4 |
| | 128. | CDR2 | AASTLDS | |
| | 129. | CDR3 | LQYAGYPYT | |
| 27 | 130. | CDR1 | RVSENIYSYLA | EGFR-D4 |
| | 131. | CDR2 | YAKTLAE | |
| | 132. | CDR3 | QHHYDIPYT | |
| 28 | 133. | CDR1 | KSSQSVLYISNQKNYLA | EGFR-D4 |
| | 134. | CDR2 | WASTRES | |
| | 135. | CDR3 | HQYFSSYT | |
| 30 | 136. | CDR1 | RASSSVSSSYLH | EGFR-D4 |
| | 137. | CDR2 | STSNLAS | |
| | 138. | CDR3 | QQYSGFPLT | |

TABLE 5
CDR sequences of exemplary antibody heavy chains.

| Clone | SEQ | | | |
|---|---|---|---|---|
| # | ID NO | | VH | Screening |
| 31 | 139. | CDR1 | GFSLTSY | EGFR-D4 |
| | 140. | CDR2 | WAGGS | |
| | 141. | CDR3 | DGSRYEGYFDY | |
| 32 | 142. | CDR1 | GYTFTNY | EGFR-D4 |
| | 143. | CDR2 | NTYTGD | |
| | 144. | CDR3 | RISTMITTWFAY | |
| 33 | 145. | CDR1 | GYTFTNY | EGFR-D4 |
| | 146. | CDR2 | NTYTGD | |
| | 147. | CDR3 | RISTMITTWFAY | |
| 34 | 148. | CDR1 | GYTFTNY | EGFR-D4 |
| | 149. | CDR2 | NTYTGD | |
| | 150. | CDR3 | RISTMITTWFAY | |
| 36 | 151. | CDR1 | ND | EGFR-D4 |
| | 152. | CDR2 | | |
| | 153. | CDR3 | | |
| 37 | 154. | CDR1 | GFTFSSF | EGFR |
| | 155. | CDR2 | SSGSS | |
| | 156. | CDR3 | QGLRYYGYAMDY | |
| 38 | 157. | CDR1 | GYTFTSY | EGFR |
| | 158. | CDR2 | NPSTGY | |
| | 159. | CDR3 | WVITTGKYFDV | |
| 39 | 160. | CDR1 | GYTFSNY | EGFR |
| | 161. | CDR2 | LPGSGR | |
| | 162. | CDR3 | QLGVRAMDY | |
| 40 | 163. | CDR1 | GYTFSNY | EGFR |
| | 164. | CDR2 | LPGSGR | |
| | 165. | CDR3 | YYRYDGGYAMDY | |
| 41 | 166. | CDR1 | GFAFSSY | EGFR |
| | 167. | CDR2 | SKGGGS | |
| | 168. | CDR3 | HAGNYAMDY | |

TABLE 6
CDR sequences of exemplary antibody light chains.

| Clone | SEQ | | | |
|---|---|---|---|---|
| # | ID NO | | VL | Screening |
| 31 | 169. | CDR1 | KSSQSLLNSNNQKNYLA | EGFR-D4 |
| | 170. | CDR2 | FASTRES | |
| | 171. | CDR3 | QQHYSTPLT | |
| 32 | 172. | CDR1 | ND | EGFR-D4 |
| | 173. | CDR2 | | |
| | 174. | CDR3 | | |
| 33 | 175. | CDR1 | RASENIYSYLA | EGFR-D4 |
| | 176. | CDR2 | NAKTLTE | |
| | 177. | CDR3 | QHYQVTPYTF | |
| 34 | 178. | CDR1 | RASENIYSYLA | EGFR-D4 |
| | 179. | CDR2 | NAKTLTE | |
| | 180. | CDR3 | QHYQVTPYTF | |
| 36 | 181. | CDR1 | KSSQSLLDSDGKTYLN | EGFR-D4 |
| | 182. | CDR2 | LVSKLDS | |
| | 183. | CDR3 | WQDTHFPQT | |
| 37 | 184. | CDR1 | RASQSIGTIIH | EGFR |
| | 185. | CDR2 | YASESIS | |
| | 186. | CDR3 | QQTNSWPLT | |
| 38 | 187. | CDR1 | RASESVDYYGNSFIH | EGFR |
| | 188. | CDR2 | RASNLES | |
| | 189. | CDR3 | QQSNEDPLT | |
| 39 | 190. | CDR1 | ND | EGFR |
| | 191. | CDR2 | | |
| | 192. | CDR3 | | |
| 40 | 193. | CDR1 | RSSKSLLHSNGITYLY | EGFR |
| | 194. | CDR2 | QMSNLAS | |
| | 195. | CDR3 | AQNLELPYT | |
| 41 | 196. | CDR1 | KASQDINSYLS | EGFR |
| | 197. | CDR2 | RANRLVD | |
| | 198. | CDR3 | LQYDEFPYT | |

TABLE 7
CDR sequences of an exemplary antibody heavy chain.

| Clone # | SEQ ID NO | | VH |
|---|---|---|---|
| 5C8VH | 199. | CDR1 | GYSFTGY |
| | 200. | CDR2 | NCYNGA |
| | 201. | CDR3 | AGIHYDYDEAWFAY |

TABLE 8
CDR sequences of an exemplary antibody light chain.

| Clone# | SEQ ID NO | | VL |
|---|---|---|---|
| 5C8VL | 202. | CDR1 | RSSTGPVIIRNYAN |
| | 203. | CDR2 | GTNNRAP |
| | 204. | CDR3 | ALWYNNHWV |

TABLE 9
Antibody heavy and light chain sequences of exemplary embodiments
provided herein.

| Clone # | SEQ ID NO | VH | SEQ ID NO | VL | Screening |
|---|---|---|---|---|---|
| 1 | 205. | QIQLVQSGPELKKPGETVKISCK | 206. | DIVMTQAAFSNPVTLGT | EGFR-D4 |
| | | ASGYTFTNYGMNWVKQAPGK | | SASISCRSSKSLLNSNGI | |
| | | GLKWMGWINTYTGEPTYADDF | | NYLYWYLQKPGQSPQL | |
| | | EGRFALSLETSASTVYLQINNLK | | LIYQMSNLASGVPDRFS | |
| | | NEDTGTYFCARFPYYGNYGAM | | SSGSGTDFTLRISRVEAE | |
| | | DYWGQGTSVTVSS | | DVGVYYCAQNLELPCT | |
| | | | | FGGGTKLEIK | |
| 2 | 207. | QIQLVQSGPELKKPGETVKISCK | 208. | QTPMTQSSTIGSASSGN | EGFR-D4 |
| | | ASGYTFTNYGMNWVKQAPGK | | RKSITSKANKDGNNNL | |
| | | GLKWMGWINTYTGEPTYADDF | | NWFQQKPGQSPKLLIY | |
| | | EGRFALSLETSASTVYLQINNLK | | GASNLESGVPARFSGSG | |
| | | NEDTATYFCARFPYYGNYGAM | | SGTDFTFTISSVEAEDV | |
| | | DYWGQGTSVTVSS | | ATYYCQQNSNFPYTFG | |
| | | | | GGTKLEIK | |
| 4 | 209. | QIQLVQSGPELKKPGETVKISCK | 210. | ND | EGFR-D4 |
| | | ASGYTFTNYGMNWVKQAPGK | | | |
| | | GLKWMGWINPYTGEPRYADDF | | | |
| | | KGRFAFSLETSASTAFLQISNLK | | | |
| | | NEDMATYFCARSVYGYWYFDV | | | |
| | | WGAGTTVTVSS | | | |
| 5 | 211. | QIQLVQSGPELKKPGETVKISCK | 212. | DIVMTQAAFSNPVTLGT | EGFR-D4 |
| | | ASGYTFTNCGMNWVKQAPGKG | | SASISCRSSKSLLHSNGI | |
| | | LKWMGWINTYTGDPTYADDFE | | TYLYWYLQKPGQSPQL | |
| | | GRFAFSLETSASTAYLQINNLKN | | LIYQMSNLASGVPDRFS | |
| | | EDTATYFCARFPYYGNYGAMD | | SSGSGTDFTLRISRVEAE | |
| | | YWGQGTSVTVSS | | DVGVYYCAQNLELPWT | |
| | | | | FGGGTKLEIK | |
| 6 | 213. | QIQLVQSGPELKKPGETVKISCK | 214. | DIVLTQSPASLAVSLGQ | EGFR-D4 |
| | | ASGYTFTHYGMTWVKQAPGKG | | RATISCRASESVDSYGN | |
| | | LKWMGWIITYTGEPTYADDFK | | SFMHWYQQKPGQPPKL | |
| | | GRFAFSLETSASTAYLQINNLKN | | LIYRASNLESGIPARFSG | |
| | | EDMATYFCARRYYGSNYFDYW | | SGSRTDFTLTINPVEAD | |
| | | GQGTTLTVSS | | DVATYYCQQSNEDPYT | |
| | | | | FGGGPTGNKTG | |
| 7 | 215. | QIQLVQSGPELKKPGETVKISCK | 216. | DIVMTQAAFSNPVTLGT | EGFR-D4 |
| | | ASGYTFTNYGMNWVKQAPGK | | SASISCRSSKSLLHSNGV | |
| | | GLKWMGWINTYTGEPTYADDF | | TYLYWYLQKPGQSPQL | |
| | | KGRFAFSLETSASTAYLQINNLK | | LIYQMSNLASGVPDRFS | |
| | | NEDTVTYFCARFPYYGNYGAM | | SSGSGTDFTLRISR VEAE | |
| | | DYWGQGTSVTVSS | | DVGVYYCAQNLELPCT | |
| | | | | FGGGTKLEIK | |
| 8 | 217. | QVTLKESGPGILQSSQTLSLTCS | 218. | QIVLTQSPALMSASPGE | EGFR |
| | | LFGFSLSTSGMGVSWIRQPSGK | | KVTMTCSASSSVSYMY | |
| | | GLEWLAHIYWDDDKRYNPSLK | | WYQQKPRSSPKPWIYL | |
| | | SRLTISKGTSSNQVFLKITSVDT | | TSILASGVPARFSGSGS | |
| | | ADTATYYCARRGNYYAMDYW | | GTSYSLTISSMEAEDAA | |
| | | GQGTSVTVSS | | TYYCQQWSSYPPTFGG | |
| | | | | GTKLEMK | |
| 9 | 219. | QVQLQQPGAELVKPGASVKLSC | 220. | DVVMTQTPLTLSVTIGQ | EGFR |
| | | KASGYTFTSYWMHWVKQRPG | | PASISCKSSQSLLDSDG | |
| | | QGLEWIGEINPSNGRTNYIEKFK | | KTYLNWLLQRPGQSPK | |
| | | IKATLTVDKSSSTAYMQLSSLTS | | RLIYLVSKLDSGVPDRF | |
| | | EDSAVYYCARYYDNDDFDYW | | TGSGSGTDFTLKISRVE | |
| | | GQGTTLTVSS | | AEDLGVYYCWQGTHFP | |
| | | | | HTFGGGPSWK | |
| 11 | 221. | LIQLVQSGPEVKKPGETVKISCK | 222. | DIQMTQSSSSFSVSLGD | EGFR |
| | | FSGYTFTNYGMTWVKQAPGKG | | RVTITCKASEDIDNRLG | |
| | | LKWMGWINTYTGEPTYADDFK | | WYQQKPGNAPRLLISG | |
| | | GRFAFSLETSASTAYLQINNLKN | | ATSLETGVPSRFSGSGS | |
| | | EDTATYFCATGGYYRYALYWG | | GKDYTLSITSLQTEDVA | |
| | | QGTLVTVSA | | TYYCQQYWSTPYTFGG | |
| | | | | GTKLEIK | |
| 12 | 223. | QVQLQQPGAELVKPGASVKLSC | 224. | ND | EGFR |
| | | KASGYTFTSYWMHWVKQRPG | | | |
| | | QGLEWIGEINPSNGRTNYIEKFK | | | |
| | | IKATLTVDKSSSTAYMQLSSLTS | | | |
| | | EDSAVYYCARYYDNDDFDYW | | | |
| | | GQGTTLTVSS | | | |
| 14 | 225. | QIQLVQSGPELKKPGETVKISCK | 226. | IND | EGFR |
| | | ASGYTFTNYGMNWVKQAPGK | | | |
| | | GLKWMGWINPYTGEPRYADDF | | | |
| | | KGRFAFSLETSASTAFLQISNLK | | | |
| | | NEDMATYFCARSVYGYWYLDV | | | |
| | | WGAGTTVTVSS | | | |
| 15 | 227. | DVQLQESGPGLMKPSQSLSLTC | 228. | EILLTQSPAIIAASPGEK | EGFR |
| | | TVTGYSITSDYAWNWIRQFPGN | | VTITCSASSSVSYMNW | |
| | | KLEWMGYISYSGNPNYNPSLKS | | YQQKPGSSPKIWIYGIS | |
| | | RISITRDTSKNQFFLQLNSVTTE | | YLASGVPARFSGSGSGT | |
| | | DTATYYCAKAATGYVMDYWG | | SFSFTINSMEAEDVATY | |
| | | QGTSVTVSS | | YCQQRSSYPLTFGAGT | |
| | | | | KLELK | |

TABLE 10
Antibody heavy and light chain sequences of exemplary embodiments
provided herein.

| Clone# | SEQ ID NO | IVH | SEQ ID NO | VL | Screening |
|---|---|---|---|---|---|
| 16 | 229. | QIQLVQSGPELKKPGETVKISCKA | 230. | ND | EGFR |
| | | SGYTFTNYGMNWVKQAPGKGLK | | | |
| | | WMGWINTYTGEPTYADDFEGRFA | | | |
| | | LSLETSASTVYLQINNLKNEDTAT | | | |
| | | YFCARFPYYGNYGAMDYWGQGT | | | |
| | | SVTVSS | | | |
| 17 | 231. | ND | 232. | DIVMTQSQKFMSTSVGDRI | EGFR |
| | | | | VSVTCKASQNVGTNVAW | |
| | | | | YQQKPGQSPKALIYSASY | |
| | | | | RYSGVPDRFTGSGSGTDF | |
| | | | | TLTISNVQSEDLAEYFCQ | |
| | | | | QYNSYPYTFGGGTKLEIK | |
| 18 | 233. | QVQLQQSGAELVRPGASVTLSCK | 234. | ND | EGFR |
| | | ASGYTFTDYEMHWVKQTPVHGL | | | |
| | | EWIGVIDPETGGTAYNQKFKGKA | | | |
| | | TLTADKSSSTAYMELRSLTSEDSA | | | |
| | | VYYCTRAVVATDYFDYWGQGTT | | | |
| | | LTVSS | | | |
| 19 | 235. | ND | 236. | DIQMTQSPSSLSASLGGK | EGFR |
| | | | | VTITCKASQDINKYIAWY | |
| | | | | QHKPGKGPRLLIHYTSTL | |
| | | | | QPGIPSRFSGSGSGRDYSF | |
| | | | | SISNLEPEDIATYYCLQYD | |
| | | | | NLWTFGGGTKLEIK | |
| 20 | 237. | EVQLQQSGAELVKPGASVKLSCT | 238. | DIVMSQSPSSLAVSAGEK | EGFR |
| | | ASGFNIKDTYMHWVKQRPEQGLE | | VTMSCKSSQSLLNSRTRK | |
| | | WIGRIDPATGNTKYDPKFQGKAT | | NYLAWYQQKPGQSPKLLI | |
| | | MTADPSSNTAYLQLSSLTSDDTAV | | YWASTRESGVPDRFTGSG | |
| | | YYCSLHYYGGTYYPMDYWGQGT | | FGTDFTLTISTVQAEDLA | |
| | | SVTVSS | | VYYCKQSYNLWTFGGGT | |
| | | | | KLEIK | |
| 23 | 239. | QIQLVQSGPELKKPGETVKISCKA | 240. | DIQMTQSPASLSASVGET | EGFR-D4 |
| | | SGYTFTNYGMNWVKQAPGKGLK | | VTITCRASENIYSYLAWY | |
| | | WMGWINTYTGEPTYADDFKGRF | | QQKQGKSPQLLVYYAKT | |
| | | AFSLETSASTAFLQINNLKNEDMA | | LAEGVPSRFSGSGSGTQFS | |
| | | TYFCTRRISTMITTWFAYWGQGTL | | LKINSLQPEDFGSYYCQH | |
| | | VTVSA | | HYGTPYTFGGGTKLEIK | |
| 24 | 241. | EVQLQQSGPELVKPGASVKISCKA | 242. | ND | EGFR-D4 |
| | | SGFSFTGYYMHWVKQSHVKSLE | | | |
| | | WIGHINPYNGVTRYNQNFKDKAS | | | |
| | | LTVDKSSNTAYMELHSLTSEDSA | | | |
| | | VYYCSRLDYDSSFDYWGQGTSVT | | | |
| | | VSS | | | |
| 26 | 243. | QVQLQQSGAELMKPGASVKISCK | 244. | PDDPSSPSSLSSYLGERVS | EGFR-D4 |
| | | ATGYTFSNYWIEWVKQRPGHGLE | | LTCRASQEISGYLNWLQQ | |
| | | WIGEILPGSGRTNYNEKFKGKATF | | KPDGTIKRLIYAASTLDSG | |
| | | TADTSSNTAYMQLSSLTSEDSAVY | | VPKRFSGSRSGSDYSLTIS | |
| | | YCAIYYRYDGGYAMDYWGQGTS | | SLESEDFADYYCLQYAGY | |
| | | VTVSS | | PYTFGGGTKLEIK | |
| 27 | 245. | QIQLVQSGPELKKPGETVKISCKA | 246. | DIQMTQSPASLSASVGET | EGFR-D4 |
| | | YGYTFTNYGMNWVKQAPGKGLK | | VTITCRVSENIYSYLAWY | |
| | | WMGWINTYTGEPTYADDFKGRF | | QQKQGKSPQLLVYYAKT | |
| | | AFSLETSASTAYLQINNLKNEDMA | | LAEGVPSRFSGSGSGTQFS | |
| | | TYFCVRRVSTMITTWFAYWGQGT | | LKINSLQPEDFGSYYCQH | |
| | | LVTVSA | | HYDIPYTFGGGTKLEIK | |
| 28 | 247. | EVQLQQSGPELVKPGASVKISCKA | 248 | NIMMTQLPSSLAVSAGEK | EGFR-D4 |
| | | SGYSFTGYYMHWVKQSHVKSLE | | VTMSCKSSQSVLYISNQK | |
| | | WIGRINPYNGATIYNQNFKDKASL | | NYLAWFQQKPGQSPKLLI | |
| | | TVDKSSSTAYMELHSLTSEDSAVY | | YWASTRESGVPDRFTGSG | |
| | | YCARDDYEGAMDYWGQGTSVTV | | SGTDFTLTISSVQAEDLAV | |
| | | SS | | YYCHQYFSSYTFGGGTKL | |
| | | | | EIK | |
| 30 | 249. | DVQLQESGPDLVKPSQSLSLTCTV | 250 | ENVLTQSPAIMSASPGEK | EGFR-D4 |
| | | TGYSITSGDSWHWIRQFPGNKLE | | VTMTCRASSSVSSSYLHW | |
| | | WMGYIHYSGSTKYNPSLKSRISIT | | FQQKSGASPKLWIYSTSN | |
| | | RDTSKNQFFLQLNSVTTEDTATYY | | LASGVPARFSGSGSGTSY | |
| | | CASWFYYAMDYWGQGTSVTVSS | | SLTISSVEAEDAATYYCQ | |
| | | | | QYSGFPLTFGAGTKLELK | |

TABLE 11
Antibody heavy and light chain sequences of exemplary embodiments
provided herein.

| Clone# | SEQ ID NO | VH | SEQ ID NO | VL | Screening |
|---|---|---|---|---|---|
| 31 | 251. | QVQLKESGPGLVAPSQSLSITCTV | 252 | DIVMTQSPSSLAMSVGQK | EGFR-D4 |
| | | SGFSLTSYGVHWVRQTPGKSLGW | | VTLSCKSSQSLLNSNNQK | |
| | | LGVIWAGGSASYNSALMSRLSITK | | NYLAWYQQKPGQSPKLL | |
| | | DNSKSQVFLKMTSLQTDDTAMY | | VYFASTRESGVPDRFIGSG | |
| | | YCARDGSRYEGYFDYWGQGTTL | | SGTDFILTISSVQAEDLAD | |
| | | TVSS | | YFCQQHYSTPLTFGTGTK | |
| | | | | LELK | |
| 32 | 253. | QIQLVQSGPELKKPGETVKISCKA | 254. | ND | EGFR-D4 |
| | | SGYTFTNYGMNWVKQAPGKGLK | | | |
| | | WMGWINTYTGDPTYADDFKGRF | | | |
| | | AFSLETSASTAYLQINNLKNEDMA | | | |
| | | TYFCARRISTMITTWFAYWGQGT | | | |
| | | LVTVSA | | | |
| 33 | 255. | QIQLVQSGPELKKPGETVKISCKA | 256 | DIQMTQSPASLSASVGET | EGFR-D4 |
| | | SGYTFTNYGMNWVKQAPGKGLK | | VTITCRASENIYSYLAWF | |
| | | WMGWINTYTGDPTYADDFKGRF | | QQRQGKSPQLLVYNAKT | |
| | | AFSLETSASTAYLQINNLKNEDMA | | LTEGVPSRFSGSGSGTQFS | |
| | | TYFCARRISTMITTWFAYWGQGT | | LKINSLQPEDFGSYYCQH | |
| | | LVTVSA | | YQVTPYTFGGGTKLEIK | |
| 34 | 257. | QIQLVQSGPELKKPGETVKISCKA | 258. | DIQMTQSPASLSASVGET | EGFR-D4 |
| | | SGYTFTNYGMNWVKQAPGKGLK | | VTITCRASENIYSYLAWF | |
| | | WMGWINTYTGDPTYADDFKGRF | | QQRQGKSPQLLVYNAKT | |
| | | AFSLETSASTAYLQINNLKNEDMA | | LTEGVPSRFSGSGSGTQFS | |
| | | TYFCARRISTMITTWFAYWGQGT | | LKINSLOPEDFGSYYCQH | |
| | | LVTVSA | | YQVTPYTFGGGTKLEIK | |
| 36 | 259. | ND | 260. | DVVMTQTPLTLSVTIGQP | EGFR-D4 |
| | | | | ASISCKSSQSLLDSDGKTY | |
| | | | | LNWLLQRPGQSPKRLIYL | |
| | | | | VSKLDSGVPDRFTGSGSG | |
| | | | | TDFTLKISRVEAEDLGLY | |
| | | | | YCWQDTHFPQTFGGGTK | |
| | | | | LEIK | |
| 37 | 261. | DVQLVESGGGLVQPGGSRKLSCA | 262 | DILLTQSPAILSVSPGERV | EGFR |
| | | ASGFTFSSFGMHWVRQAPEKGLE | | SFSCRASQSIGTIIHWYQQ | |
| | | WVAYISSGSSTIYYADTVKGRFTI | | RTNGSPRLLIKYASESISGI | |
| | | SRDNPKNTLFLQMTSLRSEDTAM | | PSRFSGSGSGTYFTLSINS | |
| | | YYCASQGLRYYGYAMDYWGQG | | VESEDIADYYCQQTNSWP | |
| | | TSVTVSS | | LTFGAGTKLELK | |
| 38 | 263. | QVQLQQSGAELARPGASVKMSCK | 264. | DIVLTQSPASLAVSLGQR | EGFR |
| | | ASGYTFTSYTMHWVKQRPGQGL | | ATISCRASESVDYYGNSFI | |
| | | EWIGYINPSTGYTNYNQKFRDKA | | HWYQQKPGQPPKLLIYR | |
| | | TLTADKSSSTAYMQLSSLTSEDSA | | ASNLESGIPARFSGSGSRT | |
| | | VYYCARWVITTGKYFDVWGAGT | | DFTLTINPVEADDVATYY | |
| | | TVTVSS | | CQQSNEDPLTFGAGTKLE | |
| | | | | LK | |
| 39 | 265. | QVQLQQSGAELMKPGASVKISCK | 266. | ND | EGFR |
| | | ATGYTFSNYWIEWVKQRPGHGLE | | | |
| | | WIGEILPGSGRTNYNEKFKGKATF | | | |
| | | TSITPFNTVFRFSGQLSSLTSEDTA | | | |
| | | VYYCSRQLGVRAMDYWGQGTSV | | | |
| | | TVSS | | | |
| 40 | 267. | QVQLQQSGAELMKPGASVKISCK | 268. | DIVMTQAAFSNPVTLGTS | EGFR |
| | | ATGYTFSNYWIEWVKQRPGHGLE | | ASISCRSSKSLLHSNGITY | |
| | | WIGEILPGSGRTNYNEKFKGKATF | | LYWYLQKPGQSPQLLIYQ | |
| | | TADTSSNTAYMQLSSLTSEDSAV | | MSNLASGVPDRFSSSGSG | |
| | | YYCAIYYRYDGGYAMDYWGQGT | | TDFTLRISRVEAEDVGVY | |
| | | SVTVSS | | YCAQNLELPYTFGGGTKL | |
| | | | | EIK | |
| 41 | 269. | EVQLVESGGGLVKPGGSLKLSCA | 270. | DIKMTQTPSSMYASLGER | EGFR |
| | | ASGFAFSSYDMSWVRQTPEKRLE | | VTITCKASQDINSYLSWF | |
| | | | | QQKPGKSPKTLIYRANRL | |
| | | WVAYISKGGGSTYYPETVKGRFTI | | VDGVPSRFSGSGSGQDYS | |
| | | SRDNAKNTLYLQMSSLKSEDTAM | | LTISSLEYEDMGIYYCLQ | |
| | | YYCVRHAGNYAMDYWGQGTSV | | YDEFPYTFGGGTKLEIR | |
| | | TVSS | | | |

TABLE 12
Antibody heavy and light chain sequences of an exemplary embodiment
provided herein.

| Clone# | SEQ ID NO | VH | SEQ ID NO | VL |
|---|---|---|---|---|
| 5C8VH | 271. | EVQLQQSGPELVKTGASVKISC | 272. | QAVVTQESALTTSPGETVTLTCRSS |
| | | KASGYSFTGYYMHWVKQSHG | | TGPVIIRNYANWVQEKPDHLFTGLI |
| | | KSLEWIGYINCYNGAINYNQKF | | GGTNNRAPGVPARFSGSLIGDKAA |
| | | KGKATFTVDTSSSTVYMQFNS | | LTITGAQTEDEAIYFCALWYNNHW |
| | | LTSEDSAVYYCVRAGIHYDYD | | VFGGGTKLTVL |
| | | EAWFAYWGQGTLVTVSA | | |

TABLE 13
Heavy and light chain CDR sequence combinations of antibody
embodiments provided herein.

| Clone# | | SEQ ID NO | VH | SEQ ID NO | VL | Screening |
|---|---|---|---|---|---|---|
| 1 | CDR1 | 1. | GYTFTNY | 37. | RSSKSLLNSNGINYLY | EGFR-D4 |
| | CDR2 | 2. | NTYTGE | 38. | QMSNLAS | |
| | CDR3 | 3. | FPYYGNYGAMDY | 39. | AQNLELPCT | |
| 2 | CDR1 | 4. | GYTFTNY | 40. | KANKDGNNNLN | EGFR-D4 |
| | CDR2 | 5. | NTYTGE | 41. | GASNLES | |
| | CDR3 | 6. | FPYYGNYGAMDY | 42. | QQNSNFPYT | |
| 4 | CDR1 | 7. | GYTFTNY | 43. | | EGFR-D4 |
| | CDR2 | 8. | NPYTGE | 44. | ND | |
| | CDR3 | 9. | SVYGYWYFDV | 45. | | |
| 5 | CDR1 | 10. | GYTFTNC | 46. | RSSKSLLHSNGITYL | EGFR-D4 |
| | CDR2 | 11. | NTYTGD | 47. | QMSNLAS | |
| | CDR3 | 12. | FPYYGNYGAMDY | 48. | AQNLELPWT | |
| 6 | CDR1 | 13. | GYTFTHY | 49. | RASESVDSYGNSFM | EGFR-D4 |
| | CDR2 | 14. | ITYTGE | 50. | RASNLES | |
| | CDR3 | 15. | YYGSNYFDY | 51. | QQSNEDPYT | |
| 7 | CDR1 | 16. | GYTFTNY | 52. | RSSKSLLHSNGVTYL | EGFR-D4 |
| | CDR2 | 17. | INTYTGE | 53. | QMSNLAS | |
| | CDR3 | 18. | FPYYGNYGAMDY | 54. | AQNLELPCT | |
| 8 | CDR1 | 19. | GFSLSTSGM | 55. | SASSSVSYM | EGFR |
| | CDR2 | 20. | YWDDD | 56. | LTSILAS | |
| | CDR3 | 21. | RGNYYAMDY | 57 | QQWSSYPPT | |
| 9 | CDR1 | 22. | GYTFTSY | 58. | KSSQSLLDSDGKTYLN | EGFR |
| | CDR2 | 23. | NPSNGR | 59. | LVSKLDS | |
| | CDR3 | 24. | YYDNDDFDY | 60. | WQGTHFPHT | |
| 11 | CDR1 | 25. | GYTFTNY | 61. | KASEDIDNRLG | EGFR |
| | CDR2 | 26. | NTYTGE | 62. | GATSLET | |
| | CDR3 | 27. | GGYYRYALY | 63. | QQYWSTPYT | |
| 12 | CDR1 | 28. | KASGYTFTSY | 64. | | EGFR |
| | CDR2 | 29. | NPSNGR | 65. | ND | |
| | CDR3 | 30. | YYDNDDFDY | 66. | | |
| 14 | CDR1 | 31. | GYTFTNY | 67. | | EGFR |
| | CDR2 | 32. | NPYTGE | 68. | ND | |
| | CDR3 | 33. | SVYGYWYLDV | 69. | | |
| 15 | CDR1 | 34. | GYSITSDY | 70. | SASSSVSYMN | EGFR |
| | CDR2 | 35. | SYSGN | 71. | GISYLAS | |
| | CDR3 | 36. | AATGYVMDY | 72. | QQRSSYPLT | |

TABLE 14
CDR sequences of antibody heavy and light chain sequences of exemplary
embodiments provided herein.

| Clone# | | SEQ ID NO | VH | SEQ ID NO | VL | Screening |
|---|---|---|---|---|---|---|
| 16 | CDR1 | 73. | GYTFTNY | 106. | ND | EGFR |
| | CDR2 | 74. | NTYTGE | 107. | | |
| | CDR3 | 75. | FPYYGNYGAMDY | 108. | | |
| 17 | CDR1 | 76. | ND | 109. | KASQNVGTNVA | EGFR |
| | CDR2 | 77. | | 110. | SASYRYS | |
| | CDR3 | 78. | | 111. | QYNSYPYT | |
| 18 | CDR1 | 79. | GYTFTDY | 112. | ND | EGFR |
| | CDR2 | 80. | DPETGG | 113. | | |
| | CDR3 | 81. | AVVATDYFD | 114. | | |
| 19 | CDR1 | 82. | ND | 115. | QDINKYIA | EGFR |
| | CDR2 | 83. | | 116. | YTSTLQP | |
| | CDR3 | 84. | | 117. | LQYDNLWT | |
| 20 | CDR1 | 85. | GFNIKDT | 118. | KSSQSLLNSRTRKNYLA | EGFR |
| | CDR2 | 86. | DPATGN | 119. | WASTRES | |
| | CDR3 | 87. | HYYGGTYYPMDY | 120. | KQSYNLWT | |
| 23 | CDR1 | 88. | GYTFTNY | 121. | RASENIYSYLA | EGFR-D4 |
| | CDR2 | 89. | NTYTGE | 122. | YAKTLAE | |
| | CDR3 | 90. | RISTMITTWFAY | 123. | QHHYGTPYT | |
| 24 | CDR1 | 91. | GFSFTGY | 124. | ND | EGFR-D4 |
| | CDR2 | 92. | NPYNGV | 125. | | |
| | CDR3 | 93. | LDYDSSFDY | 126. | | |
| 26 | CDR1 | 94. | GYTFSNY | 127. | RASQEISGYLN | EGFR-D4 |
| | CDR2 | 95. | LPGSGR | 128. | AASTLDS | |
| | CDR3 | 96. | YYRYDGGYAMDYW | 129. | LQYAGYPYT | |
| 27 | CDR1 | 97. | YGYTFTNY | 130. | RVSENIYSYLA | EGFR-D4 |
| | CDR2 | 98. | NTYTGE | 131. | YAKTLAE | |
| | CDR3 | 99. | VRRVSTMITTWFAY | 132. | QHHYDIPYT | |
| 28 | CDR1 | 100. | GYSFTGY | 133. | KSSQSVLYISNQKNYLA | EGFR-D4 |
| | CDR2 | 101. | NPYNGA | 134. | WASTRES | |
| | CDR3 | 102. | DDYEGAMDY | 135. | HQYFSSYT | |
| 30 | CDR1 | 103. | GYSITSGD | 136. | RASSSVSSSYLH | EGFR-D4 |
| | CDR2 | 104. | HYSGS | 137. | STSNLAS | |
| | CDR3 | 105. | WFYYAMDY | 138. | QQYSGFPLT | |

table 15
CDR sequences of antibody heavy and light chain sequences of exemplary
embodiments provided herein.

| Clone# | | SEQ ID NO | VH | SEQ ID NO | VL | Screening |
|---|---|---|---|---|---|---|
| 31 | CDR1 | 139. | GFSLTSY | 169. | KSSQSLLNSNNQKNYLA | EGFR-D4 |
| | CDR2 | 140. | WAGGS | 170. | FASTRES | |
| | CDR3 | 141. | DGSRYEGYFDY | 171. | QQHYSTPLT | |
| 32 | CDR1 | 142. | GYTFTNY | 172. | ND | EGFR-D4 |
| | CDR2 | 143. | NTYTGD | 173. | | |
| | CDR3 | 144. | RISTMITTWFAY | 174. | | |
| 33 | CDR1 | 145. | GYTFTNY | 175. | RASENIYSYLA | EGFR-D4 |
| | CDR2 | 146. | NTYTGD | 176. | NAKTLTE | |
| | CDR3 | 147. | RISTMITTWFAY | 177. | QHYQVTPYTF | |
| 34 | CDR1 | 148. | GYTFTNY | 178. | RASENIYSYLA | EGFR-D4 |
| | CDR2 | 149. | NTYTGD | 179. | NAKTLTE | |
| | CDR3 | 150. | RISTMITTWFAY | 180. | QHYQVTPYTF | |
| 36 | CDR1 | 151. | ND | 181. | KSSQSLLDSDGKTYLN | EGFR-D4 |
| | CDR2 | 152. | | 182. | LVSKLDS | |
| | CDR3 | 153. | | 183. | WQDTHFPQT | |
| 37 | CDR1 | 154. | GFTFSSF | 184. | RASQSIGTIIH | EGFR |
| | CDR2 | 155. | SSGSS | 185. | YASESIS | |
| | CDR3 | 156. | QGLRYYGYAMDY | 186. | QQTNSWPLT | |
| 38 | CDR1 | 157. | GYTFTSY | 187. | RASESVDYYGNSFIH | EGFR |
| | CDR2 | 158. | NPSTGY | 188. | RASNLES | |
| | CDR3 | 159. | WVITTGKYFDV | 189. | QQSNEDPLT | |
| 39 | CDR1 | 160. | GYTFSNY | 190. | ND | EGFR |
| | CDR2 | 161. | LPGSGR | 191. | | |
| | CDR3 | 162. | QLGVRAMDY | 192. | | |
| 40 | CDR1 | 163. | GYTFSNY | 193. | RSSKSLLHSNGITYLY | EGFR |
| | CDR2 | 164. | LPGSGR | 194. | QMSNLAS | |
| | CDR3 | 165. | YYRYDGGYAMDY | 195. | AQNLELPYT | |
| 41 | CDR1 | 166. | GFAFSSY | 196. | KASQDINSYLS | EGFR |
| | CDR2 | 167. | SKGGGS | 197. | RANRLVD | |
| | CDR3 | 168. | HAGNYAMDY | 198. | LQYDEFPYT | |

TABLE 16
Kinetic binding properties of EGFRD4-5C8 Fab to EGFR domain IV

| | ka (1/Ms) | kd (1/s) | KD (M) | Chi² (RU²) | U-value |
|---|---|---|---|---|---|
| EGFRD4 5C8 Fab | 1.79E+05 | 0.03047 | 1.70E−07 | 0.864 | 1 |

TABLE 17
Kinetic binding properties of EGFRD4-5C8 to EGFR domain IV

| | ka (1/Ms) | kd (1/s) | KD (M) | Chi² (RU²) | U-value |
|---|---|---|---|---|---|
| EGFR-D4-5C8 | 5.12E+05 | 2.49E-04 | 4.87E−10 | 2.22 | 1 |

TABLE 18
Kinetic binding properties of EGFRD4-5C8 to EGFR domain IV

| | ka (1/Ms) | kd (1/s) | KD (M) | Chi² (RU²) | U-value |
|---|---|---|---|---|---|
| EGFR-D4-5C8 | 6.04E+05 | 1.30E−04 | 2.14E−10 | 0.109 | 1 |

What is claimed is:

1. A recombinant nucleic acid comprising a sequence encoding a truncated EGFR (tEGFR) cell surface molecule, wherein said tEGFR cell surface molecule comprises an EGFR domain IV and does not comprise an EGFR domain III.

2. The recombinant nucleic acid of claim 1, wherein said tEGFR cell surface molecule does not comprise an EGFR domain I, an EGFR domain II, an EGFR juxtamembrane domain or an EGFR tyrosine kinase domain.

3. The recombinant nucleic acid of claim 1 or 2, wherein said tEGFR cell surface molecule is non-immunogenic.

4. The recombinant nucleic acid of claim 1 or 2, wherein said tEGFR cell surface molecule is a human tEGFR cell surface molecule.

5. The recombinant nucleic acid of claim 1 or 2, wherein said tEGFR cell surface molecule comprises an amino acid sequence having a sequence identity of at least 85% to the amino acid sequence of SEQ ID NO:276.

6. The recombinant nucleic acid of a claim 1 or 2, wherein said tEGFR cell surface molecule binds an anti-domain IV EGFR antibody.

7. The recombinant nucleic acid of claim 1 or 2, wherein said tEGFR cell surface molecule does not bind an anti-domain III EGFR antibody.

8. The recombinant nucleic acid of claim 1 or 2, further comprising a sequence encoding a chimeric antigen receptor, a T cell receptor, or a cytokine receptor.

9. The recombinant nucleic acid of claim 8, wherein said chimeric antigen receptor comprises an antibody region and a transmembrane domain.

10. The recombinant nucleic acid of claim 9, wherein said antibody region binds to a cancer-antigen.

11. The recombinant nucleic acid of claim 9, wherein said antibody region binds to CD19.

12. The recombinant nucleic acid of claim 8, wherein said sequence encoding said chimeric antigen receptor further comprises an intracellular T-cell signaling domain.

13. The recombinant nucleic acid of claim 8, wherein said cytokine receptor is IL15.

14. The recombinant nucleic acid of claim 8, further comprising a sequence encoding a self-cleaving peptidyl sequence.

15. The recombinant nucleic acid of claim 14, wherein said self-cleaving peptidyl sequence connects said sequence encoding said tEGFR cell surface molecule with said sequence encoding said chimeric antigen receptor.

16. The recombinant nucleic acid of claim 14 or 15, wherein said self-cleaving peptidyl sequence encodes a T2A peptidyl sequence, P2A peptidyl sequence, a E2A peptidyl sequence, a F2A peptidyl sequence or a 2A peptidyl sequence.

17. An expression vector comprising the recombinant nucleic acid of claim 1 or 2.

18. The expression vector of claim 17, wherein said expression vector is an adenoviral vector or a retroviral vector.

19. The expression vector of claim 18, wherein said retroviral vector is a retroviral vector.

20. A cell comprising a tEGFR cell surface molecule of claim 1 or 2 or an expression vector of claim 1 or 2.

21. The cell of claim 20, wherein said cell is bound to an anti-domain IV EGFR antibody in vitro or in vivo.

22. The cell of claim 20 or 21, wherein said cell is a T cell, a natural killer (Nk) cell or an induce pluripotent stem cell (iPSC).

23. A kit composition comprising (i) an expression vector of claim 17; and (ii) an anti-domain IV EGFR antibody or an expression vector encoding an anti-domain IV EGFR antibody.

24. The kit of claim 23, wherein said expression vector of (i) and said expression vector or said antibody of (ii) are in separate containers.

25. A method of selecting a cell expressing a tEGFR cell surface molecule, said method comprising:
(i) contacting a population of cells with a recombinant nucleic acid of claim 1 or 2 or an expression vector of claim 17, thereby forming a contacted cell population;
(ii) contacting said contacted cell population with a tEGFR binding agent, thereby forming a bound tEGFR expressing cell; and
(iii) separating said bound tEGFR expressing cell from said contacted cell population, thereby selecting a cell expressing a tEGFR cell surface molecule.

26. The method of claim 25, wherein said tEGFR binding agent is an anti-domain IV EGFR antibody.

27. The method of claim 26, wherein said antibody comprises a detectable moiety.

28. The method of claim 25, wherein said population of cells is in a subject.

29. The method of any one of claims 25-27, wherein said population of cells is in a tissue culture container.

30. A method of detecting a cell expressing a tEGFR cell surface molecule, said method comprising:
(i) contacting a population of cells expressing a recombinant nucleic acid of claim 1 or 2 or an expression vector of claim 17 with a tEGFR binding agent, and
(ii) detecting binding of said binding agent to a tEGFR cell surface molecule thereby detecting a cell expressing a tEGFR cell surface molecule.

31. The method of claim 30, wherein said tEGFR binding agent is an anti-domain IV EGFR antibody.

32. The method of claim 31, wherein said antibody comprises a detectable moiety.
